(19) United States
(12) Patent Application Publication
(10) Pub. No.: US 2022/0047811 A1
(43) Pub. Date: Feb. 17, 2022

(54) PROSTHETIC DISORDER RESPONSE SYSTEMS

(71) Applicant: David S. Goldsmith, Atlanta, GA (US)

(72) Inventor: David S. Goldsmith, Atlanta, GA (US)

(21) Appl. No.: 17/460,034

(22) Filed: Aug. 27, 2021

Related U.S. Application Data
(63) Continuation-in-part of application No. 17/329,138, filed on May 24, 2021.
Application No. 17/329,138 is a continuation-in-part of application No. 14/998,495, filed on Jan. 12, 2016, now Pat. No. 11,013,858.
Application No. 17/329,138 is a continuation-in-part of application No. 15/998,002, filed on Jun. 8, 2018.
Application No. 15/998,002 is a continuation-in-part of application No. 14/121,365, filed on Aug. 25, 2014, now abandoned.
(60) Provisional application No. 62/282,183, filed on Jul. 27, 2015.
(60) Provisional application No. 61/959,560, filed on Aug. 27, 2013.

(51) Int. Cl. A61M 5/172; A61M 5/142

(52) U.S. Cl. CPC A61M 5/1723; A61M 5/14212

(57) ABSTRACT

A fully implanted automatic disorder response control system acts as a backup “immune” system, immediately detecting and dispensing an enzyme deficient or lacking due to an inborn error of metabolism, for example, in accordance with its prescription-program. In response to a disease, the remedial action is medicinal and/or electrostimulatory. By directly pipeline-targeting agents through a closed system of fluid lines from drug reservoirs to leak-free and durable tissue connectors at the sites of disease, the system averts side effects without dispersing an agent throughout the systemic circulation, fundamentally liberalizing, while optimizing, the use of drugs. Electrostimulatory and other end-effectors available, each morbidity in comorbid disease is assigned to an arm or channel of a hierarchical control system. Symptom sensors pass data up through successively higher-level nodes to generate the cross-channel, cross-morbidity view the control microprocessor uses to command the remedial action that will optimize homeostasis.

### CROSS REFERENCE TO RELATED APPLICATIONS

This is a continuation-in-part of application Ser. No. 17/329,138, filed on May 24, 2021, which is a continuation-in-part of copending application Ser. No. 15/998,002, filed on Jun. 8, 2018, which is a continuation-in-part of U.S. application Ser. No. 14/121,365, filed on Aug. 25, 2014, which claims the benefit of U.S. Provisional Application No. 61/959,560, filed on Aug. 25, 2013. U.S application Ser. No. 17/329,138, filed on May 24, 2021, is also a continuation-in-part of U.S. application Ser. No. 14/998,495, now U.S. Pat. No. 11,013,858, filed on Jan. 12, 2016, entitled “Nonjacketing Side-entry Connectors and Prosthetic Disorder Response Systems,” which claims the benefit of U.S. Provisional Application No. 62/282,183, filed on Jul. 27, 2015. This entire disclosures of all applications in the priorirty chain are hereby incorporated by reference.

### BACKGROUND OF THE INVENTION

The methods and apparatus to be described are intended for prescription by internists, hepatologists, nephrologists, pulmonologists, cardiologists, urologists, gastroenterologists, gynecologists, and for use by endourologists, general, endocrine, oncological, neurological, pediatric cardiac, vascular, and cardiothoracic surgeons, interventional cardiologists, interventional radiologists, and veterinary specialists to allow 1. The automatic directly catheteric pipeline-targeted delivery of drugs and therapeutic or system maintenance substances to the sites of disease; 2. The semiautomatic control of compound bypass solid organ transplantation; 3. The semiautomatic placement of ductus segment replacement prostheses; as well as 4. Control nondrug therapeutic end-effectors, such as electrostimulatory, cardiac resynchronizing, thermal, and electrical assist devices in response to data transmitted to an implanted microcontroller, or in multiply comorbid disease, a hierarchical master control microprocessor executing a prescription-program responsive to data supplied by implanted sensors to continuously treat the patient while ambulatory.

### SUMMARY OF THE INVENTION

The information handling capability imparted by hierarchical control, previously used to reduce the complexity of decision-making in the fields of robotics, manufacturing, and artificial intelligence is applied to medical diagnostics and therapeutics. In a fully implanted system, sensors positioned to monitor known and predictable secondary or associated disease at the lowest local level, often cellular, input data to nodes or subcontrollers at the same level. Sensors are chosen on the basis of existing and predictable signs and symptoms. These ground level sensors pass their data to diagnostic nodes or controllers at their respective level. Therapy is primarily medicinal but may include electrostimulatory neuromodulation, for example.

At the same time, other ground level sensors strategically positioned in the same or other parts of the body, assigned to monitor the same or an associated or secondary disease process, that is, a comorbidity, likewise send disease-related data to the ground level nodes at their level. Implanted drug reservoirs are preloaded with broad spectrum pharmaceuticals effective over a range of similar, and others most effective in treating specific predictable signs and symptoms.

The nodes at the ground level, one or more in one set assigned to one morbidity and those in another set assigned to another morbidity, pass their data up to a higher cross-morbidity node that identifies medication, for example, that would address the diagnostic data for both morbidities most effectively with the least adverse effects. Where the comorbidities are more than two, the process of coordinating and integrating the indicia associated with additional comorbid disease is likewise diagnosed and passed up to higher level nodes or controllers so that at the highest level, this process integrates the data across the three morbidities.

An implanted microprocessor—the master controller—is programmed to analyze and integrate the highest level, or summary level data, formulate a therapeutic regimen consisting of the fewest drugs in the smallest doses, and where applicable, the energization of electrical therapeutic components, most likely to reinstate homeostasis across the set of comorbidities to the extent possible, then effectuate the response by actuating and metering the ‘stopcocks’ or motors at the outlets of the drug reservoirs to pipe-target the medication according to the resolution arrived at through this process. In so doing, the system reinstates the affected tissue or tissues to the most competent level of performance of which it had been capable before it became affected by disease.

The system can provide a level of performance to compensate for tissue limited by a cytological, histological, or gross anatomical deficiency or malformity that arose during development as results in an inborn error of metabolism, for example. Additionally, such a system is able to compensate for if not restore the level of function of which the structure was capable before having been degraded by disease. Attempting to exceed the level of performance of the system or structure beyond its de facto potential is specifically discounted as injurious. Accordingly, the system detects and responds to the appearance of a disorder or disease process immediately, before the patient becomes aware of it, and reacts to that emergence immediately to optimal effect, the patient ambulatory throughout. The incident can be signaled and transmitted to the clinic telemetrically.

### BRIEF DESCRIPTION OF THE DRAWING FIGURES

FIG. 1 is a schematic, or nonanatomic, representation of a fully implanted automatic ambulatory prosthetic disorder response system showing components always and a few less often needed in such a system, that shown here for the treatment of urological disorders.

FIG. 2 shows right-hand pumps in a standardized pump-pair wherein line switching using turrets allows any drug or line rotated into alignment with the pump intake by the pump intake line switching means shown as a turret to be delivered through any one line rotated into alignment with the pump outlet by the pump outlet switching means also shown as a turret but without drug vials for simplicity, the actual size of this apparatus subject to miniaturization sufficient for implantation.

FIG. 3 is a diagrammatic representation, or circuit diagram, of the control train when a single pump-pair and jacket set, size permitting, is implanted, or if not, inserted in a control, power, and/or pump body pack, shown here in the abstract as to the relationships among the parts, the train comprising a system for the hierarchical control of prescription-program in accordance with the guidelines set forth by internal medicine for immediate response to an expression of disease, and in particular, those exigent.

FIG. 4 is a diagrammatic schematic, or circuit diagram, of the interconnections in a hierarchical control system and its positioning as miniaturized for implantion inside or outside the body or relegated to a control, power, and/or pump body pack when a second pump-pair and jacket set is added to the first.

### DETAILED DESCRIPTION OF THE INVENTION

### CONCEPT OF THE INVENTION

A prosthetic disorder response system is a fully implanted interconnected network of sensors, drug reservoirs, ductus and tissue drug-releasing, electrostimulatory, thermal, or tool positioning end-connectors, and catheteric drug and medicinal solution pipelines connecting the drug reservoirs to the ductus and tissue connectors, and a controller to administer the prescription-program that implements the system.

If disease is multiply comorbid and symptomatic so that the number and/or size of components is exceptionally large, the large and/or excessive number of components is relegated to a belt-worn body pack. Instantly responsive, the system can detect the analytes associated with and strike down a genetically transmitted or predisposed disorder before the patient experiences any symptoms. That such a system is easily enabled to transmit data to a clinic by medical telemetry and that implanted power sources can be replenished by transcutaneous energy transfer is considered obvious.

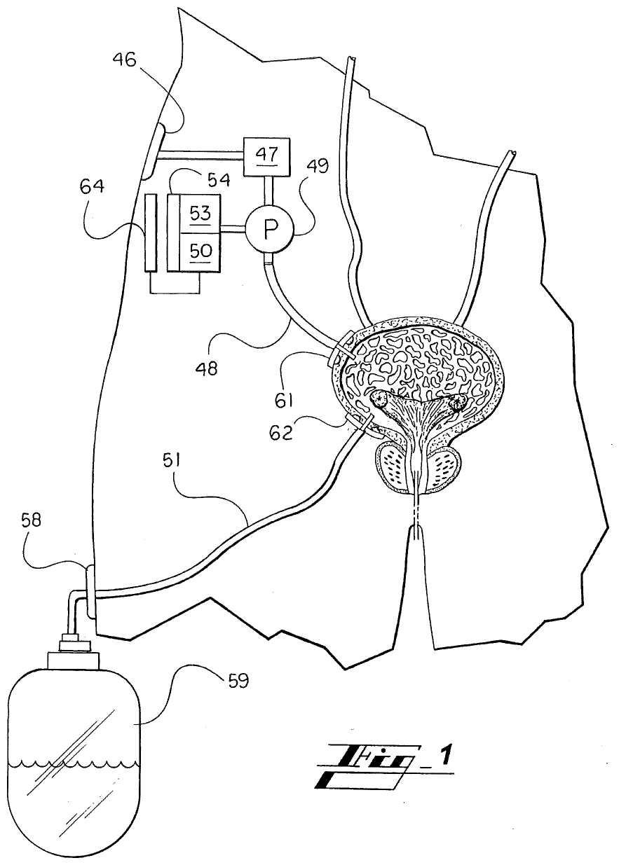

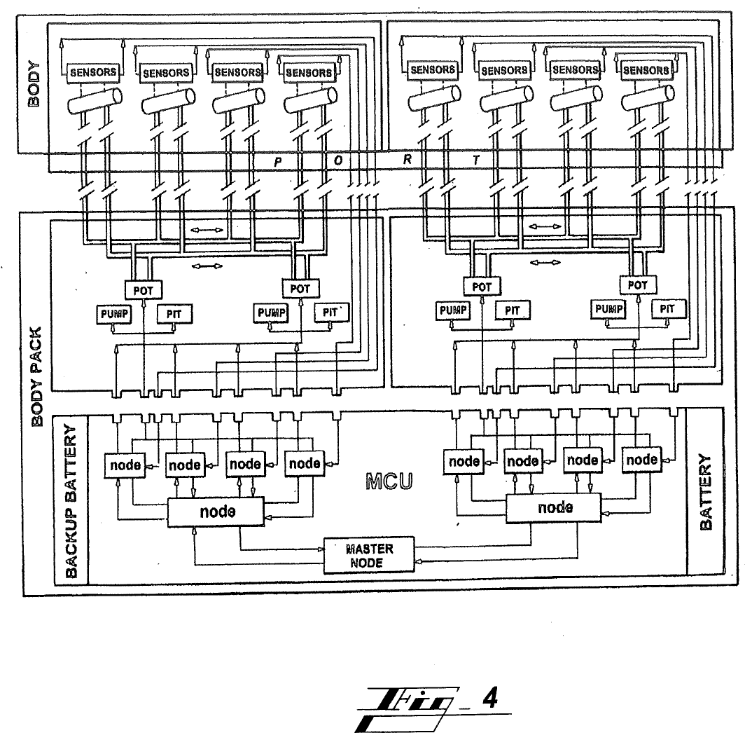

FIG. 1 shows a fully implanted system, with the only extracorporeal component a urine drainage collection bag cinched about the ipsilateral thigh. The urological application depicted in FIG. 1 is purely exemplary: any disorder or disease the subject of internal medicine might have been cited, the number and possible combinations thereof enormous. FIG. 4 clearly demarcates implanted components from those which due to their size and/or number had to be relegated to a body pack. Constituted to treat mono- or comorbid disease, such a system is negative feedback driven, sensor feedback applied as remedial action is taken to continuously indicate the instant proximity to and the realization of having arrived at the set points indicative of normalcy.

Quite apart from serving as but one element in a comprehensive automatic comorbidity response system under the control of an implanted microprocessor, no more than a nonjacketing side-entry connector, part number 61 in FIG. 1, positioned as shown, gravity fed from a commercial portacath, or mediport, 46 typically positioned subcutaneously in the pectoral area, the outlet thereof releasing a liquid antiseptic such as benzalkonium, hydrogen peroxide, or if the patient is tolerant of a stinging sensation, an alcohol or highly dilute solution of sodium hypochlorite, povidone-iodine, chlorhexidine gluconate, or hexachlorophene. released to avert repeated lower urinary tract infections as often affects women and the elderly.

Release of the antiseptic is governed by a rudimentary control system consisting of a thin film strain gauge pressure sensor incorporated into connector 61 when the bladder fills so that the roof of the detrusor commences to undulate as urge sensation begins. The control microcontroller and circuit are powered by a transcutaneously recharged button cell battery alongside portacath 46, which is replenished with the antiseptic when the symptoms of reinfection appear, to include a painful burning sensation of the urethra during urination, urinary incontinence, and frequent urination.

When the detrusor is weak so that the residual volume of urine in the bladder is excessive, a small automatically energized turbine shown and described in copending application Ser. No. 16/873,914, entitled Vascular Valves and Servovalves—and Prosthetic Disorder Response Systems, FIGS. 28 thru 30 allows thorough emptying. In copending application Ser. No. 16/873,914, the native bladder has been removed so that a prosthetic bladder is seen in its place.

Delivery into the bladder can be through any ureteral side-entry jacket or valve or through a nonjacketing side-entry connector mainline attached to the bladder roof or through an accessory channel, or sideline, of either type connector. Such a simple arrangement, consisting of no more than a portacath, catheter, and end-connector devised to remain implanted indefinitely can not be replaced by an indwelling catheter, which must not be permanently left in the body as in an accident or during rigorous exercise, it can result in a puncture or incisional injury through the substrate ductus and will gradually injure the tissue through which it had been passed.

The same minimal set of components not part of a more encompassing microprocessor-administered program to treat comorbid disease with a ductus side-entry jacket as the end connector on a vessel allows intermittent infusion of a drug directly into the circulation to achieve much quicker dispersion than were the drug taken orally, by injection, or suppository. Copending application Ser. No. 14/121,365, published as US 2016/0051806, and its continuation-in-part application Ser. No. 15/998,002, FIG. 16 shows just such an application, FIG. 21 showing use in a bypass to coronary arteries. Other monomorbid applications not requiring hierarchical control are those discutient shown and described in copending application Ser. No. 15/998,002 entitled Ductus Side-entry Jackets and Prosthetic Disorder Response Systems, FIGS. 8, 13A, 13B, and 14 thru 16.

In a hierarchical control system, these subsidiary loops are nested, each level higher in the hierarchy integrating more comprehensive information, meaning information appurtenant of an additional symptom associated with the same morbidity or with an another morbidity tracked on a different channel, or arm, of the system. This information is then combined with the information on the channel of reference to integrate the two, and thus allow a determination as to the best resolution for the two taken together. At the highest level, the master control microprocessor commands remedial action that would best serve the reinstatement of normal homeostasis across the sum of morbidities.

Since remedial actions are isolated from one another by pipeline and electrical command targeting, interaction among the morbidities will seldom if ever be simple and direct but rather secondary consequences of the interdependence among organs, tissues, and bodily systems attributable to neuroendocrine and autonomic interactions able to bypass direct physically isolated targeting. The type of end-effectors used follows from the disorder or disorders to be treated, and can include those electrostimulatory or otherwise neuromodulatory along with a rechargeable power source.

The number of potential configurations for such a system equals the number of serious chronic conditions and the combinations and permutations thereof, so that to describe a comprehensive set of specific systems in specific terms would require many years. In such a fully automatic and fully implanted system, it is essential that all end connectors—ductus side-entry jackets, nonjacketing side entry connectors, vascular valves, and inline coupling jackets—fastened to vessels or to tissue surfaces can be depended upon not to leak, dislodge, fracture, break down, foul, clog, or otherwise fail, if not for the life of the patient, then for many years.

Such a system can be implanted to support any conventional surgical procedure that calls for the dispensing of supportive medication and follow-up monitoring as well as to initiate remedial action as necessary. Unless presenting complications, ordinarily straightforward and routine stitching procedures such as herniorrhaphies and excisional procedures such as varicocelectomies are not considered to warrant the placement of such a system.

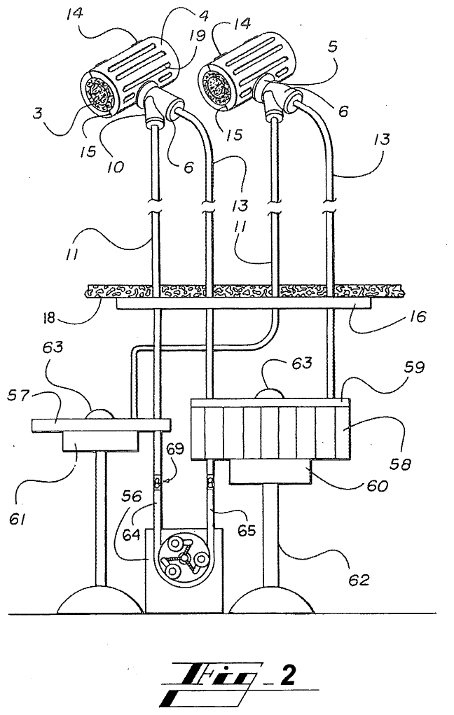

Other common tissue end connectors include the ductus side-entry jacket placed in surrounding relation to a substrate native ductus such as a blood vessel, of which two appear here at the top of FIG. 2, having previously appeared as FIG. 32 in copending application Ser. No. 15/998,002, entitled Ductus Side-entry Jackets and Prosthetic Disorder Response Systems, of which such ductus connecting jackets were the subject. The choice of this figure is purely exemplary, any other organ or tissue having been selectable. A prosthetic disorder response system can be used to diagnose and treat any organ or tissue.

Vascular valves and servovalves as shown in copending application Ser. No. 16/873,914, entitled Vascular Valves and Servovalves—and Prosthetic Disorder Response Systems, are modified, hence, similar to, side-entry jackets but differ in incorporating means for dividing the flow-through cross section between either of two outlets. There are two types—those driven by a solenoid, which suddenly and fully extend a diversion chute ito the lumen to divert all flow into a takeoff passageway, and those driven by a servomotor, which allow the gradual extension and retraction of the diversion chute to apportion flow between either of the two outlets.

Another type of vascular valve is the inline coupling jacket. This is a one-time destructive periductal collar placed at either end of a prosthetic replacement segment to replace the native segment when too diseased or malformed to be repaired. One example is a neonate born with a connective tissue disorder that has resulted in a very large aneurysm, interrupted aortic arch, or coarctation along the thoracic aorta for which no repair would prove durable and capable of growth. Another is the more familiar abdominal aortic aneurysm usually presented in an adult in whom an endoprosthesis poses the risk of an endoleak.

Under the restorative force of its spring hinges, the inline coupling jacket cuts through the ends of the native segment and rotates the prosthesis into its place in one continuous action so quick as to not interrupt the flow of blood through the substrate vessel. Both vascular valves and inline coupling jackets require clearance to place, and this can usually be attained with the aid of retractors. Where the distal end of a ductus plunges into anatomy too tight to access, such as the great vessels upon departing from the heart, the distal jacket is placed as far distally as possible and the ductus distal theretro if susceptible to structural failure, is exceptionally protected with an endoprosthesis.

application Ser. No. 15/932,172, listed first below led to the realization that the periductal collars described could be connected by catheteric piping from implanted drug reservoirs to nidi under the control of an implanted microcontroller to release drugs and electrical or wireless lines directly connected to the microcontroller in a targeted manner. This avoids side effects and the need for the considerably larger dosing required for systemic circulation with its exposure of nontargeted tissue, and eliminates the need for repeated invasive procedures to accomplish treatment for which the tools have been prepositioned and are automatically or remotely controlled.

The complete dependency of such a system upon ductus and tissue connectors that will remain intact, not migrate, leak, break or malfunction, and incorporate means for eliminating biofilm, clot, crystal, pathogens, and injury to the substate ductus, as well as the desirability of showing uses for such connectors in cooperative arrangements under automatic diagnostic and therapeutic control prompted the next three applications—this because existing connectors not only omitted such capabilities but would actually work counterproductively to induce the degeneration of the substrate ductus or tissue so that the more these fine structures were exposed, the better.

Rather existing connectors were ‘dumb,’ not only in omitting these structural requirements but in failing to provide immediate accessibility for the control of diagnostic and therapeutic measures dependent upon fluid and electrical access. In contrast, the connectors shown in the last three applications allow the direct body surface port-to connector transcatheteric passage of miniature cabled devices such as an angioscope, laser, or linear or rotary thrombectomizer through the connector and into the substrate ductus, and the direct pipeline targeting of medication.

Diagnostic sensors, some specified below and many classified by analyte in copending application Ser. No. 16/873,914, entitled Vascular Valves and Servovalves—and Prosthetic Disorder Response Systems, can be incorporated into the connectors. When necessary, different nonmedicinal therapeutic mechanisms, such as electrostimulatory, laser, thermal, and radiation-emitting, can be mounted to if not incorporated into connectors controlled through wire- or wireless radio-transmitted commands.

Moreover, as delineated in copending application, Ser. No. 16/873,914, entitled Vascular Valves and Servovalves—and Prosthetic Disorder Response Systems, ductus side-entry jackets can be made adjustable in apportioning flow between either of two outlet passageways, making possible the performing of several new surgical procedures, to include the administration of medication, semiautomaticly and therefore accessible to a larger and more widely distributed number of surgeons.

1. Integrated System for the Infixion and Retrieval of Implants, Ser. No. 15/932,172, largely concerned with the treatment of vascular and hematogenously disseminated disease;

2. Ductus Side-entry Jackets and Prosthetic Disorder Response Systems, Provisional application Ser. No. 61/959,560 filed on 27 Aug. 2013 and Nonprovisional application Ser. No. 15/998,002, concerned mostly with the design of blood and urine outlets into and inlets from catheteric fluid pipelines serving as shunts or bypasses;

3. Nonjacketing Side-entry Connectors and Prosthetic Disorder Response Systems, Ser. No. 14/998,495, now U.S. Pat. 11,013,858, occupied mostly with connectors fastened to the surface of tissue rather than in surrounding relation to a ductus; and

4. Vascular Valves and Servovalves—and Prosthetic Disorder Response Systems, Ser. No. 16/873,914, concerned with the design of valves that allow control over the flow of blood or urine.

Substantiation that in compressing and completely enclosing the fine vessels and nervelets entering and departing the adventitia of the substrate ductus or tissue actually promotes degenerative disease is prominent in the medical literature (see, for example, Arun, M. Z., Üstünes, L., Sevin, G., and Özer, E. 2015. “Effects of Vitamin C Treatment on Collar-induced Intimal Thickening,” Drug Design, Development, and Therapy 9:6461-6473; Kivelä, A., Hartikainen, J., and Ylä-Herttuala, S. 2012. “Dotted Collar Placed Around Carotid Artery Induces Asymmetric Neointimal Lesion Formation in Rabbits without Intravascular Manipulations,” BMC [BioMed Central] Cardiovascular Disorders 12:91; Nobécourt, E., Tabet, F., Lambert, G., Puranik, R., Bao, S., Yan, L., Davies, M. J., Brown, B. E., Jenkins, A. J., Dusting, G. J., Bonnet, D. J., Curtiss, L. K., Barter, P.J., and Rye, K. A. 2010. “Nonenzymatic Glycation Impairs the Antiinflammatory Properties of Apolipoprotein A-I,” Arteriosclerosis, Thrombosis, and Vascular Biology 30(4):766-772; Reel, B., Oktay, G., Ozkal, S., Islekel, H., Ozer, E. Ozsarlak-Sozer, G., Cavdar, Z., Akhisaroglu, S. T., and Kerry, Z. 2009. “MMP-2 and MMP-9 [matrix metalloproteinases]-alteration in Response to Collaring in Rabbits: The Effects of Endothelin Receptor Antagonism,” Journal of Cardiovascular Pharmacology and Therapeutics 2009 14(4):292-301; Kerry, Z., Yasa, M., Sevin, G., Reel, B., Yetik Anacak, G., and Ozer, A. 2005. “Diverse Effects of Calcium Channel Blockers in the Collar Model,” Acta Cardiologica 60(5):493-499; Nicholls, S. J., Dusting, G. J., Cutri, B., Bao, S., Drummond, G. R., Rye, K. A., and Barter, P. J. 2005. “Reconstituted High-density Lipoproteins Inhibit the Acute Pro-oxidant and Proinflammatory Vascular Changes Induced by a Periarterial Collar in Normocholesterolemic Rabbits,” Circulation 111(12):1543-1550; Donetti, E., Baetta, R., Comparato, C., Altana, C., Sartore, S., Paoletti, R., Castano, P., Gabbiani, G., and Corsini, A. 2002. “Polymorphonuclear Leukocyte-myocyte Interaction: An Early Event in Collar-induced Rabbit Carotid Intimal Thickening,” Experimental Cell Research 274(2):197-206; Bruijns, R. H. and Bult, H. 2001. “Effects of Local Cytochalasin D Delivery on Smooth Muscle Cell Migration and on Collar-induced Intimal Hyperplasia in the Rabbit Carotid Artery,” British Journal of Pharmacology 134(3):473-483; Crauwels, H. M., Herman, A. G., and Bult, H. 2000. “Local Application of Advanced Glycation End Products and Intimal Hyperplasia in the Rabbit Collared Carotid Artery,” Cardiovascular Research 47(1):173-182; Sözmen, E. Y., Kerry, Z., Uysal, F., Yetik, G., Yasa, M., Ustünes, L., and Onat, T. 2000. “Antioxidant Enzyme Activities and Total Nitrite/Nitrate Levels in the Collar Model. Effect of Nicardipine,” Clinical Chemistry and Laboratory Medicine 38(1):21-25; Herman, A., Matthys, K., Van Hove, C., Kockx, M., and Bult, H. 1999. “Oxidized Low-density Lipoprotein Enhances Intimal Thickening and Alters Vascular Reactivity,” Verhandelingen (Koninklijke Vlaamse Academie voor Geneeskunde van België) [Proceedings of the Belgian Royal Academies of Medicine] 61(1):19-38; Kerry, Z., Yasa, M., Akpinar, R., Sevin, G., Yetik, G., and 5 others 1999. “Effects of Nicardipine on Collar-induced Intimal Thickening and Vascular Reactivity in the Rabbit,” Journal of Pharmacy and Pharmacology 51(4):441-447; Yasa, M., Kerry, Z., Yetik, G., Sevin, G., Reel, B., and 5 others 1999. “Effects of Treatment with FK409 [((+/−)-(E)-4-ethyl-2-[(E)-hydroxyimino]-5-nitro-3-hexenamide], a Nitric Oxide Donor, on Collar-induced Intimal Thickening and Vascular Reactivity,” European Journal of Pharmacology 374(1):33-39; Van Put, D. J., Van Osselaer, N., De Meyer, G. R., Andries, L. J., Kockx, M. M., De Clerck, L. S., and Bult, H. 1998. “Role of Polymorphonuclear Leukocytes in Collar-induced Intimal Thickening in the Rabbit Carotid Artery,” Arteriosclerosis, Thrombosis, and Vascular Biology 18(6):915-921; Arthur, J. F., Yin, Z .L., Young, H. M., and Dusting, G. J. 1997. “Induction of Nitric Oxide Synthase in the Neointima Induced by a Periarterial Collar in Rabbits,” Arteriosclerosis, Thrombosis, Vascular Biology 17(4):737-740; Baetta, R., Donetti, E., Comparato, C., Calore, M., Rossi, A., Teruzzi, C., Paoletti, R., Fumagalli, R., and Soma, M. R. 1997. “Proapoptotic Effect of Atorvastatin on Stimulated Rabbit Smooth Muscle Cells,” Pharmacological Research 36(2):115-121; De Meyer, G. R., Van Put, D. J., Kockx, M. M., Van Schil, P., Bosmans, R., Bult, H., Buyssens, N., Vanmaele, R., and Herman, A. G. 1997. “Possible Mechanisms of Collar-induced Intimal Thickening,” Arteriosclerosis, Thrombosis, and Vascular Biology 17(10):1924-1930; Matthys, K. E., Van Hove, C. E., Kockx, M. M., Andries, L. J., Van Osselaer, N., Herman, A. G., and Bult, H. 1997. “Local Application of LDL [low density lipoprotein] Promotes Intimal Thickening in the Collared Carotid Artery of the Rabbit,” Arteriosclerosis, Thrombosis, and Vascular Biology 17(11):2423-2429; Ustünes, L., Yasa, M., Kerry, Z., Ozdemir, N., Berkan, T., Erhan, Y., and Ozer, A. 1996. “Effect of Verapamil on Intimal Thickening and Vascular Reactivity in the Collared Carotid Artery of the Rabbit,” British Journal of Pharmacology 118(7):1681-1688; Van Put, D. J., Van Hove, C. E., De Meyer, G. R., Wuyts, F., Herman, A. G., and Bult, H. 1995. “Dexamethasone Influences Intimal Thickening and Vascular Reactivity in the Rabbit Collared Carotid Artery,” European Journal of Pharmacology 294(2-3):753-761); Arthur, J. F., Dusting, G. J., and Woodman, O. L. 1994. “Impaired Vasodilator Function of Nitric Oxide Associated with Developing Neo-intima in Conscious Rabbits,” Journal of Vascular Research 31(4):187-194; Reckless, J., Fleetwood, G., Tilling, L., Huber, P. A., Marston, S. B., and Pritchard, K. 1994. “Changes in the Caldesmon Isoform Content and Intimal Thickening in the Rabbit Carotid Artery Induced by a Silicone Elastomer Collar,” Arteriosclerosis and Thrombosis 14(11):1837-1845.

Accordingly, these preliminary applications laid the groundwork for fully implanted automatic ambulatory prosthetic disorder response systems for which an unavoidable prerequisite are ductus and tissue connections that are secure, supported by accessory channels that directly pipeline-target maintenance solutions and drugs to the junctions and the tissue to which they are connected, and withal dependable for years if not for the life of a younger patient. Ductus connected thus can therefore thwart clogging due to a buildup of clot, crystal, or biofilm as well as eradicate the pathogen that deposited it. All devices and procedures described in these applications have been devised for use without the need to arrest blood flow or general anesthesia.

The availability of such systems will not only facilitate medicine and surgery as currently practiced but retroactively prompt and implement the adaptation of existing as well as recommend new therapeutic, diagnostic, and surgical techniques. In more advanced applications, a prosthetic disorder response system can considerably automate transplantation and prosthesis replacement procedures, notably, compound bypass solid organ transplantation—t seamless switching of the blood supply and drainage from the native organ of the recipient to and through that of the donor, transferring the graft organ from the circulatory system of the donor into that of the recipient.

Compound bypass, or switch, solid organ transplantation is described and illustrated in copending applicaton Ser. No. 16/873,914, entitled Vascular Valves and Servovalves—and Prosthetic Disorder Response Systems. Semiautomatic vascular segment replacement pertains to larger blood vessels and their branches. Both solid organ recipient to donor switching and prosthetic ductus segment insertion require not only vascular valves and servovalves and inline coupling jackets respectively, but all other tissue and ductus connectors in the disorder response system must be designed no to leak, migrate, abrade neighboring tissue, or otherwise cause complications for years if not for the life of the patient.

Currently, the major drawback to organ transplantation when any other treatment would represent a half way measure is the lifelong need for immunosuppressive medication. For this reason, transplantation is said to represent the replacement of one disease with another -—nuisance in a competent and a menace in an incompetent patient. Another deterrent is the frequently limited life of the graft organ before it must be replaced.

However, the automatic targeted release of immunosuppressive, other maintenance drugs, and vascular valve-implemented anoxia-free compound bypass procedure making it possible to directly transfer the graft organ from the circulatory system of the donor into that of the recipient should materially extend the life of the transplant, dispelling these deterrents often cited to justify leaving the patient in a less than optimal condition. Equipped with rejection analyte sensors, postoperatively and unceasingly, the automatic system immediately detects, signals, and releases no more medication than is needed to suppress the advancement of rejection.

In this way, the familiar comment that an organ transplant only exchanges one disease for another is considerably reduced if not dispelled. Heart transplantation using a compound bypass technique is fundamentally superior to any conventional method from every standpoint. By placing the donor on life support before dying and directly transferring the graft organ from the circulatory system of the donor into that of the recipient, the heart is never subjected to the shock of death, circulatory arrest, or ischemia-reperfusion injury.

In that far-off day when tissue engineers gain the ability to generate a fully functional replacement organ from stem cells harvested from the patient, the problem of how to insert the new organ in place of the old with minimal trauma and then how best to support the graft organ following placement will remain. Then, genetic matching having been omitted from the problem, the relatively low trauma of a sudden switch transplantation implemented with solenoid driven valves, followed if and only if necessary, by the continuous monitoring and medicinal support of a fully implanted automatic prosthetic disorder response system, will assure the suppression of atherosclerotic degeneration and graft organ durability.

Until then, genetic matching will remain the major cause for late if not acute rejection, a problem that metered compound bypass, or switch, transplantation with followup by an implanted response system can considerably ameliorate. Metered compound bypass, or switch, transplantation uses continuously variable servovalves adjusted gradually by the implanted control microprocessor to sustain the relatively silent donor-recipient reciprocal cross circulatory microchimerization that without preprocedural cross transfusions, for example, is momentary in a sudden switch transplant.

By extending the reciprocal cross circulation in a sudden switch transplantation, metered switch transplantation assists to induce immune tolerance between the donor and the recipient gradually enough to minimize if not avert a rejection reaction more likely to ensue when exposure to alien tissue arises by abrupt confrontation. This subdued or relaxed approach may be preceded, accompanied, or preceded by the conventional administration of immune tolerance inducing medication to include the gradual exchange of tissues between the donor and the recipient.

With donor life support initiated prior to death and spontaneous circulation and breathing sustained, this gradual transfer of the graft organ from the circulatory system of the donor into that of the recipient avoids the severe reaction systemic inflammatory response, that is, the polypeptide mediator release syndrome, infusion reaction, or ‘cytokine storm,’ and systemic inflammatory if not hperinflammatory response syndrome associated with organ failure and death will have been considerably suppressed and probably prevented from degrading the prospective graft.

Broadly, eliminating this deterioration reduces the multiple obstacles of transplantation to one of immune tolerance, and the metered switch, or compound bypass, method, described in copending application Ser. No. 6/873,914, entitled Vascular Valves and Servovalves—and Prosthetic Disorder Response Systems, reduces this remaining problem as well. Essentially, the graft organ is spared the trauma, and therewith, the ‘realization,’ that its host had died and that it had been transferred into an alien milieu.

In fact, surgical procedures controlled peri- and midoperatively to partially if not completely automate their execution are simplified as to remove these from the exclusive purview of a relatively small number of highly skilled and experienced specialists. Brought within the compass of general surgeons, access to such support, especially for rural and less developed world populations, is considerably increased. Given this virtually universal applicability that encompasses a profuse number of complex problems of internal medicine and surgery, examples cited must be limited, sufficient information provided to make it apparent how a system to treat a specific disease or combination thereof would be configured.

In monomorbid disease, the controller is a microcontroller; while in comorbid disease, it will usually be a microprocessor administering a hierarchical control program in which the microprocessor acts as the master controller executing a program in which each component morbidity is assigned to a channel, or arm, in a rising ladder of nodes, or levels of diagnostic data collection, with cross-node data integration accomplished at each level among the channels. Rising up a level then calls for integrating the information associated with the additional morbidity with that accumulated at a lower level for the morbidities as distinct.

That is, at subordinate levels in the hierarchy, the data in the different arms at the same level are cross-compared to identify the optimal treatment across the larger number of morbidities at that level. Each rising step extends this integration to include an additional channel. The master controller then integrates the information arriving up through the subordinate levels in the hierarchy and issues commands to achieve the most efficacious result for treating the combination of conditions. This does not, however, equate to the dispersal of drugs through the systemic circulation. Indeed, each release of a drug or other therapy is directly pipeline-targeted and can be fully isolated from any others, thus eliminating side effects due to this indiscriminate dispersal.

Despite the tightest targeting of drugs, the organs and tissues of the body represent a fully integrated system, so that the release of drugs must take into account not direct exposure but rather sequelary, or secondary, interaction among organs and tissues. Cardiorenal and cardiohepatic conditions, for example, represent just two of the common conditions of interaction attesting to the interdependence of all parts of the body mediated by the autonomic and endocrine as well as the circulatory system. Physiological interdependence thus is distinct from the similar affectation of distributed nervous tissue due to centralized defects in genes that govern neuroendocrine function as pertains to the paragangliomas in both the stomach and carotid bodies, for example, as well as defects in genes with pleiotropic substrates remote from one another.

FIG. 1 here, previously published as FIG. 12A in U.S. Pat. No. 11,013,858, entitled Nonjacketing Side-entry Connectors and Prosthetic Disorder Response Systems, provides a schematic representation of a more complex system which includes a transdermal (or transcutaneous), battery charging secondary coil 64 and transdermal charging circuitry 50, as well as a battery 54, surface port 46 here positioned subcutaneously in the pectoral region, drug storage reservoir or reservoirs 47, miniature reversible pump 49, microprocessor 53, and drug delivery pipeline, or drugline 48, leading to side-entry connector 61.

Depending upon the condition or conditions to be treated, other components might include electrostimulatory or otherwise neuromodulatory, as well as warming, cooling, and/or pumping devices, for example. The positioning of such a system is for response to serious chronic, chronic intermittent, or episodic conditions or for surgical administration and/or surgical follow-up. The system can also continuously monitor and respond to any chronic disorder where the only surgical factor consists of emplacement of the system itself accomplished endoscopically through ‘keyhole’ incisions of a few centimeters, drug delivery pipelines and electrical conductors tunneled subcutaneously and around, to avoid strangling, viscera with the aid, for example, of an ultrasound handpiece.

A totally implanted automatic prosthetic disorder response system can control the execution and then support a number of surgical procedures, some, such as a heart transplant, critical for survival in patients of all ages, and another the replacement of large vasculature so congenitally malformed that given the rate of growth in a neonate, no conventional means for its repair will prove satisfactory for more than a short time. Were the defect corrected once and for all, or at least for a period of years, the child would not be plagued and repeatedly debilitated with reoperations. The advancement this bodes for heart transplantation, executed using the compound bypass, or switch, method under the automatic control of the disorder response system warrants emphasis. Usually, the need for a new heart—or rather a part thereof—is due to ventricular failure of the native heart, necessitating replacement of the ventricles.

Unlike a kidney transplant, for example, where the graft organ is left intact and orthotopically positioned in place of the original or heterotopically, a heart transplant is really a hybrid repair that cuts off and takes the working part of the donor heart and removes the defective part of the recipient heart, then stitches the working parts of each together to make a working heart. A prosthetic disorder response system could be used to support this or any other conventional procedure; however, its emergence enables superior methods that allow the direct targeting of drugs to nidi without exposing unintended tissue, for example.

Where the conventional approach is to cut off the ventricles from the donor heart to replace the ventricles of the recipient heart, the compound bypass technique eliminates the need to cut into either heart. Thus, using a conventional technique, the donor heart is not used intact. Rather, both the native and donor hearts are more or less cut in half and then stitched together, so that the ‘transplant’ actually retains much of the recipient heart and consists of both. The need for immunosuppressives is no less applicable following a compound bypass transplant as set forth here. Incision into both the donor and recipient hearts not only traumatizes both severely, but disallows continuity of perfusion in either.

Circulation through the hearts necessarily withheld during this procedure, the reinstatement of perfusion causes further trauma to both hearts in the form of ischemia-reperfusion injury, strongly suspected to reappear as the cardiac allograft vasculopathy that almost inevitably results in graft failure. The limited life of the transplanted heart is to be expected: in conventional heart and other solid organ transplantation, the donor or graft organ is excised from its natural milieu after the host has died so that circulation has stopped with the organ then stored thus.

When the donor and recipient are not already at or readily transported to the same location, the cessation in circulation following remote harvesting and subsequent loss of perfusion may be ameliorated during transport of the graft organ with the aid of a normothermic ex vivo perfusion machine; however, the shock and trauma of death, excision, and interruption if not the loss of perfusion cannot be reversed. When the donor is discovered after having died, it is better to deliver the body intact rather than the graft organ.

The new combination of severely traumatized and imperfectly matched hearts must then be protected at the expense of safety to the body as a whole through the administration of immunosuppressives. Conventional heart transplantation does not completely remove the native heart but rather replaces the ventricles with those taken from a healthy heart. This fixes the native and donor tissues in immediate interdependent contact not effectively inseparable in the event of rejection or infection. That the immunosuppressives are dispersed throughout the circulation is yet another major insult.

A heart transplant with the support of such a system is a genuine transplant that orthotopically replaces or—in a compound bypass type double heart transplant—heterotopically supplements the native with a donor or accessory organ not sewn onto and therefore treatable separately from that native. Given the new option of removing an imperfectible heart from the recipient in its entirety and replacing not just its ventricles but the entire intact heart with a good intact one, an attempt to repair such a heart, along with use of mechanical assist devices, is properly relegated to a bridging action to sustain the patient until a good heart becomes available.

Unsurprisingly, a heart transplant accomplished using conventional methods usually requires retransplantation within one, sometimes up to two decades, during which the patient must take immunosuppressives that produce an increased susceptibility to infection. If this regimen is not followed, the transplant will be rejected, and without the support of a mechanical assist device and retransplantation, the patient will die. Until then, infection, rejection, or both are fully capable of killing him. If surgical repair is a half way measure, then as currently practiced, heart transplantation is also a half way measure.

Fundamental improvements in the transplantation procedure and its follow-up treatment, will, however, improve the results of a heart or any other solid organ transplant to become fully satisfactory. With a prosthetic disorder response system, the administration of medication is automatic—in the case of a metered compound bypass heart transplant, having been administered by the same system that conducted the operation—so that cognitive impairment, negligence, or contumacy cannot result in a failure to adhere to the prescription. As with other medication best kept from unaffected parts of the body such as chemotherapeutic, the totally implanted prosthetic disorder response system tightly targets the bulk of such medication, sparing the rest of the body increased susceptibility to infection, making such a totally implanted automatic drug delivery system a major advancement in its own right.

The convenience and noninvasiveness of oral and every other conventional form of drug delivery is often gained at the cost of indiscriminate dispersal throughout the circulatory system that exposes nontargeted tissue which may lead to adverse side effects and requires dosage levels high enough to compensate for this degree of dilution. Absent an intrinsic affinity such as that of iodine for the thyroid gland, alternative routes are less convenient, but subject to the same shortcomings. In contrast, medical surgery consists of prepositioning prescription-responsive sensors and drug or other therapy-releasing components in support of a medical diagnosis. To emplace such a system is invasive, but falls far short of major surgery.

The aim in medical surgery is to position if not preposition drug delivery sites so as to best target the nidi or origins of chronic medical conditions. The goals in such positioning or prepositioning are procedural optimization and durability. In continued postprocedural treatment, the aims are immediacy and efficacy of response—to lie in wait for and counteract the disorder or disease while nascent through a direct multiply resourced attack to overwhelm and obliterate it in a targeted manner with exposure of nontargeted tissue to the drugs employed eliminated.

Such an implanted system can be prescribed on the basis of a genetic analysis at birth to counteract a predictable or highly probable disorder well before the appearance of symptoms and the condition has the opportunity to advance from the subclinical to the clinical. Ideally, the disorder or disease is counteracted before the patient even becomes aware of it. Such an automatic response system can be placed to treat any existing condition and can be supplemented and reprogrammed as necessary to deal with an additional or a different condition with little more than negligible dissection required.

Placed to dispel an inborn error of metabolism, or another internal medical disorder, or in support of a surgical procedure, the system can be updated to deal with any change in patient status and has a place in the treatment of any but relatively simple and straightforward diseases and procedures. Emplaced preoperatively, the system can not only provide postprocedural monitoring and treatment, but as pertains to compound bypass solid organ transplantation and the replacement of irreparably congenitally malformed vasculature in a neonate with inline coupling jacket-connected tie-line prostheses, can administer the procedure. Where hard wires are best avoided, electrical sensory and command signals can be communicated by wireless transmission and energy transfer to component-inmate batteries recharged by transcutaneous energy transfer.

This also allows for growth from infancy to adulthood. To extend with growth, fluid pipelines can be fluted or configured much as accordion bellows, elastic, and coiled, for example. The means for accomplishing these applications have been described and illustrated in copending application Ser. No. 16/873,914, entitled Vascular Valves and Servovalves—and Prosthetic Disorder Response Systems. In time, failure to back up a more complex operative procedure susceptible to any of a number of adverse sequelae with a totally implanted automatic ambulatory prosthetic disorder response system will be a half way measure.

Not simple, for example, is diabetes, and the latest means for its treatment to include continuous glucose monitors do nothing to monitor or respond to emergent, or nascent, complications nephropathic, cardiovascular, infective, especially mucocutaneous fungal, neurological, ophthalmic, as well as several others. The most obvious application for such system is one placed to automatically release medication in a prescription nonadherent patient and/or one prevented from optimal medication unless directly pipe-targeted due to side effects.

Ideally, symptoms are averted before they appear even when the patient is otherwise engaged, ambulatory, and oblivious. The release is of insulin through a ductus side-entry jacket directly into the portal vein, sensor inputs indicating complications treated as separate arms in the hierarchical control system of which each prompts the release of medication to the respective nidus or nidi, the master controller managing the dispensing of medication to optimize the overall efficacy.

Despite being fundamentally inferior to a targeted technique which avoids indiscriminate dispersal and requires minor invasiveness to place, noninvasive is considered the ‘gold standard,’ even in the treatment of serious chronic disease. In this, neither an entirely nor a partially systemic dose is discounted where appropriate. In that it merely relinquishes the use of superior technology to dispel misguided apprehensions, its use when avoidable is actually irresponsible—half way, indecisive and inconclusive.

The advantages of such a system basic and significant, to refrain from recommending its implementation where appropriate, to instead misrepresent as an enormity the minor surgery required to place it, and persist in prescribing oral medication despite the risk of side effects, or knowingly prescribe less effective medication to avoid the side effects, that is, the conscious use of half way measures when a more effective response is available can be achieved through the physical targeting of each drug accedes to malpractice. Half way measures rooted in unjustified hesitancy routinely result in substandard treatment and discomfort.

Concern for an accusation of malpractice is one cause for hesitancy, unfamiliarity with the new another. Ultimately, there is no serious disease or disorder that would not materially benefit from the surveillance and immediate response of such a system, readily implantable in any patient regardless of age, mental competency, or the ability and inclination to adhere to a prescription. In the 1950s, pediatric cardiac surgery could aspire to no more than save the life of the baby. A genuine repair through heart transplantation precluded by the certainty of rejection, only inadequate repair was possible. The emergence of immunosuppressives represented a major step forward.

The advent of heart transplantation materially improved matters, in that the incorporation of a normal heart made possible the normal development of the child, which attempts at surgical repair and/or the use of mechanical assist devices still cannot achieve. However, the lack of adequate surgical technique, of less traumatizing means for harvesting, preserving, and transplanting the donor heart without effectively strangling and severely wounding, then severely traumatizing the recipient heart to merge the two would critically impair the durability of the operation.

Transplantation still a half way measure, often the baby would survive for decade, maybe a dozen or so sick and unpromising years and then die anyway, a fate likely facilitated by the impairment inflicted both by the technique employed at the outset as well as weakened immunity resulting from the indiscriminate dispersal of immunosuppressives throughout the systemic circulation rather than by graduating the concentration of and distributing the drugs for optimal effectiveness. Today, to repair a congenitally severely malformed heart with the object of accomplishing no more than to save the life of the baby despite the substandard and shortened life to follow is a ruinous half way measure not to be tolerated.

Given the trauma to both donor and recipient organs—harvesting with extensive incision and anoxia, then the press of the immune system to eradicate the graft organ, for the average heart transplant to survive for a decade and sometimes longer rates as a welcome but decidedly counterintuitive outcome. Viewed from this perspective, solid organ transplantation as practiced today still represents a half way measure. The severely congenitally malformed heart still poses a choice between either of two half way measures—surgical repair that avoids the equal if not greater trauma of transplantation with the risk of rejection but is unable to initiate normal pulsatile, or pulsatant circulation, or a heart transplant that poses the constant threat of rejection.

Pediatric cardiac surgery allows relatively minor to moderate malformities—mostly interventricular defects—to be repaired. However, the complete and sufficient repair of complex defects such as a univentricular heart remains elusive and is not likely ever to become possible. Without normal circulation, all tissues and organs in the body will fail to achieve normal development. The deficits following an optimal repair may be less than conspicuous, and a transplant meticulously supported using conventional means should last for years.

Nevertheless, in either case, the life to follow is likely to be sick and relatively short. Performed as a bridge to heart transplantation, procedures such as the bidirectional Glenn, the hemi-Fontan, Norwood, and fenestrated Fontan are life-sustaining. However, to then leave the patient over a longer term than necessary without a transplant despite the fact that the consequence of impaired circulation is maldevelopment of the body as a whole an abuse, the settling for a half way measure when use instead of the latest technology would allow a durable and proper repair.

‘Half way measure’ is also an appropriate characterization of the current means for the repair of severe congenital malformities of the thoracic aorta and by extension, the aorta in its entirety. Here the situation is much like that of the severely malformed heart—there are numerous inadequate techniques and prostheses, but there is no good and durable repair for such a defect. The only way to fix the aorta once and for all is to replace it or the defective segment with a durable prosthesis that will not dehisce, migrate, or leak, will propagate the pulse, and will grow with the patient.

Whether to support a relatively minor repair of the aorta following conventional repair or the replacement of the aorta or a segment thereof with a prosthesis of the kind described in copending application Vascular Valves and Servovalves—and Prosthetic Disorder Response Systems, the placement of lines to deliver medication directly into the repaired aorta or substances to simulate endothelial function in the prosthesis is an improvement upon convention.

Even if there is no frank coarctation or interruption, the baby born with a connective tissue disorder of a severity conducive to the development of an aneurysm should be alleviated of this life-long threat once and for all at the outset. Much as the advent of immunosuppressives rendered heart transplantation feasible leaving the inadequate repair of the severely malformed neonatal heart a half way measure, the advent of means for the replacement or repair of the carotids with the placement of a pipeline to directly and automatically deliver a topical medication such as a statin into either carotid, leaves a conventional endarterectomy needlessly risky and a half way measure.

Unlike the carotids in an adult, which congenitally functional have usually become impaired due to the buildup of plaque, the thoracic aorta congenitally malformed to the extent that it cannot be dependably repaired once and for all is not simply degraded and restorable to a previously normal condition. Rather repair in this case is by replacement with a strong and pulse propagating prosthesis sufficiently expandable to accommodate growth from infancy to adulthood.

Copending application Ser. No. 16/873,914, entitled Vascular Vales and Servovalves—and Prosthetic Disorder Response Systems, shows a fabric devised to accommodate growth in vascular prostheses to replace the great or smaller vessels of a tensile strength that eliminates the possibility of a failure in strength equivalent to an aneurysm. As delineated in that application, these are tie-lines connected to the ends of the native vessel at either end by inline coupling jackets which semiautomatically replace a segment along or the entrety of a vessel without the need for clamping the blood supply.

While tissue engineering should eventually provide such vessels, current efforts have not produced any that support endothelial function and growth. For use in an infant, the ability to expand with rapid growth all the way to adulthood is crucial to eradicate the need for numerous reoperations and the iterative draining this inflicts. Electrical conductors can also be conformed for considerable extension, infrequent if any limitations thereto overcome through the use of carrier frequency distinguished wireless reception incorporated into the end connectors or effectors. While completely normal endothelial function is not imparted, function to the extent of simulating the secretion of vasodilators and vasoconstrictors in step with the data provided by prosthetic chemo- and vasopressor sensors is easily accomplished by direct pipeline release into the prosthesis.

Whereas the carotids require a repair in the form of an endarterectomy and rarely replacement, a thoracic aorta congenitally malformed to the extent that is cannot be adequately repaired requires replacement. However, owing to the current state of the art, it cannot be replaced by a strong, expandable, and pulse-responsive prosthesis with branches but must instead be reconstructed or repaired, such as through a combination of a proximal prosthesis and distal intraluminal or endovascular prosthesis as in an ‘elephant trunk’ repair. The fabric described in copending application Ser. No. 16/873,914 will solve this problem.

Repair or replacement of the thoracic aorta seldom if ever afford the opportunity to retain the native aortic bodies. The carotids are not more important for detecting hypoxia or hypercapnia and sustaining ventilatory drive to dispel these than is the aortic bodies; however, albeit seldom, the thoracic aorta is subject to much more extensive congenital malformities which demand extensive reconstruction that is denervating and destroys the chemo- and baroreception of the carotid bodies, so that the severely constricted or aneurysmal thoracic aorta, especially when likely to reaneurysm, is best replaced with a prosthesis.

In comparison, the carotids are rarely severely malformed, requiring instead the removal of acquired atherosclerotic plaque in adults. Repairable through a carotid endarterectomy, the unitlateral preservation of the carotid bodies is adapted to more readily than is the loss of the aortic bodies. Moreover, repair rather than the removal and replacement or extensive repair of the carotids allows switching bypass to facilitate the endarterectomy as well as its healing and followup monitoring and therapy.

When an ability to switch from the native structure to a permanent part time bypass prosthesis—as can be provided for the carotids—cannot be provided, as following replacement of the thoracic aorta, the prosthesis nevertheless incorporates at least one drug delivery line, or accessory channel, to allow the controller to command the direct delivery into the prosthesis of medication responsive to the need therefor as indicated by the sensors also incorporated into the prosthesis. A prosthetic disorder response system can provide drug delivery lines and the circuitry to govern their use under any circumstances, from the need to counteract an inborn error of metabolism, to the site of a conventional surgical procedure, to the site of procedures and devices which the response system made possible.

Since replacement does not require the ability to switch between the native and prosthetic passageways, the aorta is replaced with nonswitchable inline coupling jackets as the end connectors of the prosthesis to the native stumps, whereas repairable without excision of the carotid bodies, the carotids are repaired with the aid of a bypass that necessitates the use of switchable valves.

That is, both the aortic and carotid procedures consist of implanting a prosthesis, but in the case of the aorta, nonswitchable end connectors are used where in the case of the carotids, switchable end connecting valves are used. Both the switchable bypass type prosthesis connected with valves and the nonswitchable permanent replacement prosthesis connected with inline coupling jackets shown in copending application Ser. No. 16/873,914,entitled Vascular Valves and Servovalves—and Prosthetic Disorder Response Systems, FIGS. 25A and 25B, eliminate the use of a shunt and the possibility for an interruption in the flow of blood.

The factors that govern justification for providing an implanted switchable bypass and the cost thereof to be accorded a repair include:

1. The facilitated healing of the procedure when afforded the benefit of a switchable bypass.

2. The odds of failure, requiring an automatically activated ‘bailout’ backup.

3 The odds of partial failure due to known deficits of the repair compared to native function as necessitate a backup capable of compensating for the shortfall.

4. The consequences of a partial or complete failure.

5. The space available to implant the components required.

A compound bypass type heterotopic double heart transplant may be thought of as a switchable bypass.

While refusal to settle for half way measures once superior measures—such as for replacing the defective thoracic aorta—have become available is incontestable, the reciprocal thereof—never replacing a native organ that can be repaired to satisfaction—such as the carotids—is also valid. Means for the semiautomatic replacement of any segment along or the entirety of the aorta or any other larger vessel—consisting of inline coupling jacket substrate vessel end connectors and an expandable span connecting the end connectors—are described and illustrated in copending continuation in part application entitled Vascular Valves and Servovalves—and Prosthetic Disorder Response Systems.

More generally, a genetic analysis of the neonate if not the fetus indicative of any significant metabolic defect or defects should be evaluated for correction first through gene therapy, and if appropriate, the emplacement of an automatic response system to counteract the condition or conditions indicated using gene or other means for elimination or suppression of the condition before it emerges. However, unlike the heart for which no satisfactory artificial replacement that would continue to perform dependably over the life of even an elderly patient exists, to serve in the relatively passive role of an artery, it is possible to provide a durable prosthesis and to do so without the need to interrupt the circulation in order to insert the prosthesis.

Unlike a transplant, a prosthesis poses no risk of provoking an immune response greater than a readily suppressed foreign body reaction. Moreover, a prosthesis is unsusceptible to infection, and requires no blood supply. Much like heart transplantation in the 1950s in having to await the advent of immunosuppressives to progress to basic sufficiency—but still demanding fundamental improvements even today—another procedure that awaits major improvement before it accedes to maturity is carotid endarterectomy. These procedures, transplantation, essential in the treatment of end stage heart failure as well as to save the lives of neonates with severe malformities of the heart from a sick and short life, and an endarterectomy to clear the carotids of atheromatous plaque, are among those needed most frequently.

A conventional carotid endarterectomy risks the escape of thromboembolic debris, and no more than a momentary interruption in the throughflow of blood can result in anoxia, both eventualities posing the possibility of stroke. In contrast, neither the three-armed prosthesis described in copending application 16/873,914, entitled Vascular Valves and Servovalves—and Prosthetic Disorder Response Systems using inline coupling jackets at the end of each arm to suddenly replace a severely diseased, possibly carotid body-malignant carotid with a Y-shaped prosthesis, nor that using valves or servovalves to bypass the carotids during and after endarterectomy shown in FIGS. 25A and 25B risk either of these threats.

The nonvalved device has inline coupling jackets at the end of each arm of the Y-shaped prosthesis and at least one accessory channel connected to the internal carotid arm, accessory channels to the common carotid and external carotid arms provided according to the overall condition of the patient. The nonvalved device is used to accomplish the replacement of the native carotid by both severing the native structure at each of its three ends and rotating the Y-shaped prosthesis into position a single step. With either the nonvalved or the valved device, bypassed blood flow is closed off from any detritus, and the flow of blood is never interrupted. The valved, or switchable device is used when the native carotids, as in a routine endarterectomy, are preserved with the carotid bodies intact, making the ability to automatically switch back to the native structure when the patient undergoes exertion beneficial.

The direct targeting of maintenance solutions into the prosthesis prevents the accumulation of thrombus or debris along the inner walls and the direct delivery into the internal carotid of medication such as anticonvulsive, antipsychotic, anxiolytic, and so on to the brain, passage through the blood brain barrier possibly necessitating the addition of mannitol in inverse proportion to the age of the patient. Lithium to treat bipolar disorder is kept from the kidneys. Drugs that require conversion in the liver are administered in their post-liver passage active metabolized form. Most surgical procedures performed on the carotids are not to remove these but only to remove plaque, so that the carotid bodies are retained.

The three-armed carotid bypass device with valve, that is, switchable connectors described and shown in FIGS. 25A and 25B in copending application 16/873,914 allows endarterectomy with bypass and retention of the native carotids and carotid bodies with no interruption in throughflow, after which the device can be removed, or left in place as a prosthesis. Left in place, switching between the repaired native carotids and the prosthesis provides several advantages, to include access to the respiratory function of the carotid bodies on an as needed basis, while bypassing blood flow around the native structures during healing, diagnosis, and therapy.

The need for bilateral removal of the carotid bodies is dangerous in eliminating ventilatory drive in response to hypoxemia (see, for example, Wasserman, K. 1978. “The Carotid Bodies: Pathologic or Physiologic?,” Chest 73(5):564-566) resulting in the loss of consciousness and the possibility of serious trauma due to falling. carotid body tumors, usually extra-adrenal paragangliomas, are rare, those bilateral rarer, and those malignant rarer still; however, for those with the bilateral loss of the carotid bodies, their condition is more than sufficiently disabling to demand attention.

Replacement of the native carotids is with the nonvalved, inline coupling jacket end-connected, prosthesis, not the native-to-bypass and bypass-to-native switchable valved device, of which the special value is lost when the native carotids have been removed. In a unilateral removal, the nonvalved device is used on the side of the removal, and where the contralateral carotid is endarterectomized, for example, the valved device is best left in place to allow switching.

Postoperative bypassing with a valved embodiment relieves the healing carotids from needless stress, clears these for treatment or diagnosis, and allows the control microcontroller when signaled by a hypoxemia or hypercapnia sensor to automatically switch to the native carotids to stimulate ventilatory drive, averting syncope and the threat of injury. Fortunately, unlike the bilateral loss of both carotid bodies, the need to remove a carotid is more often unilateral. A bilateral carotid endarterectomy to remove plaque leaves the carotids and the carotid bodies in place, and the loss or extensive reconstruction of the aorta which results in its denervation and loss of the aortic bodies all allow for adaptation and compensation over time.

Nevertheless, certain conditions make it necessary to excise bilateral carotid body tumors resulting in the loss of ventilatory drive responsive to hypoxemia (see, for example, Chen, Y., Li, Y., Liu, J., and Yang, L. 2020. “The Clinical Characteristics and Outcomes of Carotid Body Tumors in Chinese Patients—A STROBE [STrengthening the Reporting of OBservational studies in Epidemiology]-compliant Observational Study,” Medicine (Baltimore, Md.) 99(3):e18824; Butt, N., Baek, W. K., Lachkar, S., Iwanaga, J., Mian, A., and 5 others 2019. “The Carotid Body and Associated Tumors: Updated Review with Clinical/Surgical Significance,” British Journal of Neurosurgery 33(5):500-503; Lin, B., Yang, H., Yang, H., and Shen, S. 2019. “Bilateral Malignant Paragangliomas in a Patient: A Rare Case Report,” World Neurosurgery S1878-S8750(18)32954-1; Hoang, V. T., Trinh, C. T., Lai, T. A. K., Doan, D. T., and Tran, T. T. T. 2019. “Carotid Body Tumor: A Case Report and Literature Review,” Journal of Radiology Case Reports 13(8):19-30; Khurana, A., Mei, L., Faber, A. C., Smith, S. C., and Boikos, S. A 2019. “Paragangliomas in Carney-Stratakis Syndrome,” Hormone and Metabolic Research 51(7):437-442; Anand, J. and Singh, J. P. 2018. “Bilateral Sporadic Carotid Body Tumors—A Rare Case Report,” Radiology Case Reports 13(5):988-992; Burgess, A., Calderon, M., Jafif-Cojab, M., Jorge, D., and Balanza, R. 2017. “Bilateral Carotid Body Tumor Resection in a Female Patient,” International Journal of Surgery Case Reports 41:3879-391; Ghali, M. G. Z., Srinivasan, V. M., Hanna, E., and DeMonte, M. 2017. “Overt and Subclinical Baroreflex Dysfunction after Bilateral Carotid Body Tumor Resection: Pathophysiology, Diagnosis, and Implications for Management,” World Neurosurgery 101:559-567; Han, L. V., Chen, X., Zhou, S., Cui, S., Bai, Y., and Wang, Z. 2016. “Imaging Findings of Malignant Bilateral Carotid Body Tumors: A Case Report and Review of the Literature,” Oncology Letters 11(4):2457-2462; Nicholas, R. S., Quddus, A., Topham, C., and Baker, D. 2015. “Resection of a Large Carotid Paraganglioma in Carney-Stratakis Syndrome: A Multidisciplinary Feat,” British Medical Journal Case Reports 2015:bcr2014208271; Rosa, M. and Sahoo, S. 2008. “Bilateral Carotid Body Tumor: The Role of Fine-needle Aspiration Biopsy in the Preoperative Diagnosis,” Diagnostic Cytopathology 36(3):178-180).

In a prosthetic disorder response system where the carotids with carotid bodies had to be removed, hypoxemia or hypercapnia would be readily detected by one or more tiny implanted pulse oximeter sensors, for example, from which low value inputs would signal the controller to directly electrostimulate the breathing centers in the medulla and pons.

System Control of Multidrug Delivery Systems

According to the present concept, a pharmacist-programmer enters this into a program whereby each drug is provided in response to the conditions sensed. To deliver drugs automatically and adjust the dosing, the prescription, an adaptive drug delivery program, responds to diagnostic sensor feedback under the control of a medically adapted hierarchical (nodal, nested-levels) ‘intelligent’ hard real-time ‘pathfinding’ control system (references on hierarchical control are provided below).

Less complex than is comorbid, much less multimorbid disease that necessitates a divide-and-conquer approach, monomorbid disease will usually not require multiple level, or hierarchical, control administered by an implanted microprocessor serving as the master controller that integrates the pre-processed data of subordinate nodes or controllers and issues drug release commands. In a comorbid diagnostic and therapeutic system, microcontrollers descend from the level of a monomorbid master node to a node subordinate to the master microprocessor.

Automatic ambulatory disorder response systems to monitor, diagnose, and treat relatively straightforward monomorbid disease and the nodes or controllers subordinate to the master control microprocessor in a hierarchical control system are usually highly miniaturized, large scale integrated single chip microcontrollers such as those produced by Microchip Technology's PIC [Peripheral (or Programmable, Interface Controller or Programmable Intelligent Computer] line and Atmel, for example. For implantation, these are housed to provide thermal insulation and a chemical barrier to prevent contact with tissues.

Since microcontroller and multicore microcontroller input pins are needed to set the program, additional pins to input collateral functions such as those from sensors placed to signal changes in medical conditions and outputs to execute the program, and a significant storage capacity needed to record potential changes, the microcontroller assigned to any given drug rerservoir outlet pump-pair plug-in pump-pack such as those depicted in copending application Ser. No. 14/121,365, entitled Ductus Side-entry Jackets and Prosthetic Disorder Response Systems, FIGS. 29, 31, 32, and 36 or the implanted equivalent thereof in a distributed set of pump-packs under unified control must provide a number of pins and performance capacity consistent with industrial multicore microcontrollers.

The PICoPLC program ladder logic editing, simulating, and compiling tool can generate native code for 8-bit and 32-bit microcontrollers, such as the Parallax, Inc. Propeller and Microchip Technology PIC16 central processing units from a ladder diagram, effectively gaining in a microcontroller a level of integrative capability associated with programmable logic controllers (see, for example, M. Rafiquzzaman 2018. Microcontroller Theory and Applications with the PIC18F; New York, N.Y.: Wiley; Haddad, N. K. 2017. Microcontroller System Design Using PIC18F Processors, Jacksonville, Fla.: IGI Global; Dogan Ibrahim 2014, PIC Microcontroller Projects in C: Basic to Advanced (for PIC18F), London, England: George Newnes Limited;. Sanchez, J. P and Canton, M. P 2006. Microcontroller Programming: The Microchip PIC, Boca Raton, Fla.: Chemical Rubber Company Press; lovine, J. 2000. PIC Microcontroller Project Book, New York, N.Y.: TAB [Technical Author's Bureau] Books Publishing Company.

For these and other microcontrollers, further reduction in size and power consumption are afforded through discretization, whereby the continuous steam of data is converted into a sequence of data points with sufficient accuracy preserved for control purposes. Sensor inputs that justify proportional-integral-derivative closed loop feedback from implanted sensors may be discretized.

Conversion of closed loop physiological or life-sign input data into a sequence of points then overcomes the need for an expensive and larger programmable logic controller able to perform the ongoing calculation essential to control the continuous process as such (see, for example, Uzunovic, T. and Turkovic, I. 2012. “Implementation of Microcontroller Based Fuzzy Controller,” 6th Institute of Electrical and Electronics Engineers International Conference on Intelligent Systems, Sofia, Bulgaria, available at Institute of Electrical and Electronics Engineersxplore. Institute of Electrical and Electronics Engineers.org; Velagic, J., Kuric, M., Dragolj, E., Ajanovic, Z., and Osmic, N. 2012. “Microcontroller Based Fuzzy-PI [Proportional-Integral] Approach Employing Control Surface Discretization,” 20th Mediterranean Conference on Control and Automation, Barcelona, Spain, available at Institute of Electrical and Electronics Engineersxplore.Institute of Electrical and Electronics Engineers.org; Avery, S., Gracey, C., Graner, V., Hebel, M., Hintze, J., LaMothe, A., Lindsay, A., Martin, J., and Sander, H. 2010. Programming and Customizing the Multicore Propeller Microcontroller: The Official Guide, New York, N.Y.: McGraw-Hill; Nass, M. 2010. “Xilinx Puts ARM [advanced reduced instruction set computation machine] Core into its FPGAs [field-programmable gate arrays],” Embedded, available at http://www.embedded.com/electronics-products/electronic-product-reviews/embedded-tools/4115523/Xilinx-puts-ARM-core-into-its-FPGAs; McConnel, T. 2010. “ESC—Xilinx Extensible Processing Platform Combines Best of Serial and Parallel Processing,” Electronic Engineering Times, available at http://www.eetimes.com/document.asp?doc_id=1313958; Cheung, K. 2010. “Xilinx Extensible Processing Platform for Embedded Systems,” available at http://fpgablog.com/posts/arm-cortex-mpcore/; Kanagaraj, N., Sivashanmugam, P., and Paramasivam, S. 2009. “A Fuzzy Logic based Supervisory Hierarchical Control Scheme for Real Time Pressure Control,” International Journal of Automation and Computing 6(1):88-96; Keckler, S. W., Olukotun, K., and Hofstee, H. P. 2009. Multicore Processors and Systems, New York, New York: Springer; Scanlan, D. A. and Hebel, M. A. 2007. “Programming the Eight-core Propeller Chip,” Journal of Computing Sciences in Colleges 23(1):162-168). Linear stage motors usally steppers, other type motors are not to be excluded.

When used for the direct pipeline-targeted delivery of drugs into vessels through side-entry jackets or into a volume of tissue by nonjacketing side-entry connectors (references cited above under Cross Reference to Related Applications), a primary object in the use of and is to implement drug delivery aligned to network feedback. When the data is complex, it is processed to include data reduction and integration by means of a hierarchical control system.

Where diagnostic data alone would leave it to the diagnostician to translate the data into remedial action drug delivery could not be immediate, pharmacokinetically and pharmacodynamically optimized, nor unerringly targeted, automatic control that breaks down, integrates, and compares the data up through levels that progressively coordinate more encompassing cross morbidity data makes possible diagnosis and therapy that is optimized in each of these regards. If the patient is not to be bedridden or the condition is chronic, a number of needled catheters cannot be used. Ductus side-entry connection jackets afford secure connection to the ductus, and in so doing, enable not just single point direct-to-ductus drug delivery, but the implementation of such a prosthetic supplementary disease-process compensation system.

The side-entry ductus side-entry jackets, nonjacketing side-entry connector, and drug reservoir outlet pump-pair sets to be described thus make possible the targeted delivery of drugs through automatic response that is immediate. Were the condition to exceed the range of adjustment for which the system had been set, the exigent readings can be transmitted to a clinician able to adjust the dosing by remote control

Sensors that must not be allowed to lose in sensitivity due to the predictable development of a sensor-enveloping fibrous capsule are shielded from this eventuality with an accessory channel to deliver a dissoluting drip such as dilute hydrochloric acid or a dilute hypochlorite such as bleach to occasionally wet that part or parts of the sensor outer surface which must be afforded a clear ‘sight line.’ If a minute amount per drip is satisfactory and would serve to reduce the implant load, a centralized reservoir is used to release the dissoluting agent to all sensors that need it. Since the probability is high that the agent will be reacted to as an irritant, the dissolutive agent is alternated with an irritant counteractant.

In the case of hydrochloric acid, the counteractant is sodium bicarbonate. So that implicative data is always identified to its location, and remedial measures, usually drugs, can be immediately targeted to the site or sites, both sites of primary disease, and sites likely to present progressive, sequelary, or an associated continuation of the disease process, disease analyte-detecting sensors such as report a disproportionate concentration in T or B cells or sites of elevated temperature indicative of malignancy are always mapped to the control system. The location of each sensor is included in the output data. With known disease, the sensors chosen should be closely selective for the detailed analytes as earmarks diagnostic for the disease.

In what may be best described as a ‘lying-in-wait’ posture, sensors are positioned where cellular or otherwise low-level symptoms are most likely to appear first. So that the patient need not undergo another endoscopic procedure to position additional sensors to monitor secondary disease predictable ab initio, the additional sensors, drug reservoirs, and pipelines are placed at the outset. Where the odds for the emergence of any one of a number of equally possible sequelary diseases are equal, if possible, broad spectrum analytes diagnostic for either are monitored, blood and urine draws, for example, used to secure a positive identification.

Reduction in the number and volume of drugs supports eliminatation of the need for the patient to wear of a paracorporeal, usually belt-worn, drug reservoir and pack, whether needed for power, control, or to house pumps. Paracorporeal, or belt-worn packs are discouraged as inviting tinkering and interfering with a fully closed skin implementation. To the extent possible, an automatic ambulatory disorder response system should preserve the outward integrity of a normal body to include freedom from the need to wear mandatory equipage especially an indisposible body pack. In most instances, the number of comorbidities to be addressed and the drugs needed to deal with these will be few enough that the drug reservoirs and/or power requirement will not compel the need for this impediment.

If possible, to allow full, that is, closed-skin implantation, all components of the automatic response system are miniaturized, and implemented with large scale integrated microelectronics. As shown in copending application Ser. No. 16/873,914, entitled Vascular Valves and Servovalves—and Prosthetic Disorder Response Systems, FIGS. 26A and 26B, small drug reservoirs are replenished through closed-skin body surface ports by multiple nozzle injection syringes such as that shown in FIG. 27A or by multiple nozzle jet injectors such as that shown in FIG. 27B.

Each nozzle can deliver the same or a different drug to replenish different reservoirs. A port described in copending application Ser. No. 14/121,365 incorporates means other than a conventional skin button or skin barrier for averting infection and instability. The port provides as many entry holes as the number of drug reservoirs and pipelines feeding into accessory channels requiring periodic drug replenishment.

Mechanical rather than electronic embodiments depicted in copending application, Ser. No. 15/998,002, entitled Ductus Side-entry Jackets and Prosthetic Disorder Response Systems FIGS. 29, and 31 thru 36 were intended to convey a clearer pictorial representation than might be provided using schematic diagrams and because the system and because implementation was in terms of these components being stored in a power and control pack. In most cases, it is preferable both from the standpoint of freedom from the need to wear a pack and inaccessibility to the inquisitive to use electronic components equivalent to those mechanical shown where the entire disorder response system is inside the body.

With either type device, injection is through a multiple opening mediport- or portacath-type port positioned subcutaneously (subdermally) in the pectoral region without an opening to the outside. Such a port allows the replenishment of drugs through the skin and a self-resealing cover membrane through which the injection needle or needles are passed. Tiny tattooed dots overlying the subdermal port indicate how the injector is to be aligned to assure the release of each drug into the proper reservoir.

A body surface port with an opening to the outside is used only when miniature cabled devices to include angioscopes, excimer lasers, and various diagnostic probes, chemical and imaging, diagnostic and/or therapeutic, are to be freely pass through to the nidus from outside the body, or when placed to a side of the mons pubis, to allow the passage of urine into a collection bag. Prostheses that bypass the lower urinary tract are fully described and illustrated in copending application Ser. No. 16/873,914, entitled Vascular Valves and Servovalves—and Prosthetic Disorder Response Systems. Broadly, cautious diagnosis and drug selection in preparation for system placement best fosters system efficiency.

In a prosthetic disorder response system placed to optimize the overall health of a patient with multiple comorbidities, the urinary tract—or in the absence of a urinary tract, equivalent removal of toxins from the blood of nocuous substances—is represented as one of the channels of control in a hierarchical control system. To optimize magnetic separation, or extraction, the electromagnets are made as light in weight as enclosure within a thin but tough nonallergenic plastic case and windings of silver wire, which provide greater field strength for the weight, will allow. If necessary, the micro- or nanoparticle carrier particles bonded to the target analyte or analytes are formulated to incorporate silicon-iron crystal, materially increasing their magnetic susceptibility.

Ancillary factors as pertain to specific pharmaceuticals, tissue expansion to create an intracorporeal pocket to hold onein or more drug reservoirs or another component of the implanted drug distribution system, the use of different electric current discharge patterns from the semicircular anchoring needles of nonjacketing tissue connectors in combination with different drugs passed through the drug pipeline connected to the substrate tissue by the nonjacketing side-entry connectors, and numerous other related topics, are covered in Nonprovisional application Ser. No. 14/998,495, entitled Nonjacketing Side-entry Connectors and Prosthetic Disorder Response Systems filed on 12 Jan. 2016, and Nonprovisional application Ser. No. 15/998,002 entitled Ductus Side-entry Jackets and Prosthetic Disorder Response Systems, both filed under 35 U.S.C. 119(e) on 25 Aug. 2014. In the latter application, FIGS. 29 and 31 thru 36 provide a literal pictorial

For medical use, such jackets and nonjacketing connectors must remain leak-free, nonmigrating or nondislodgeable, nondeformable, nonfracturing, and not injurious to the substrate or neighboring tissue indefinitely. Moreover, for pediatric use, these must adapt to growth over a period of years. Copending application Ser. No. 14/121,365 also addressed means for securely fastening catheteric lines, injection needles, and electrodes, for example, to native ductus through a small entry wound for the long-term treatment of chronic conditions, and delineated the assignment of channels or axes of control in a hierarchical control system to different organs or organ systems in the treatment of comorbid disease, for example.

Detailed information on the structure and function of the different type connectors used in a prosthetic disorder response system will be found in the copending applications specified. The foregoing applications also provide detailed information on the treatment of many diseases and methods of treatment, to include nephrogenic systemic fibrosis, diabetic gastroparesis, solid tumors benign and malignant, the direct and point-blank targeting of tumors internal to delicate organs that to excise surgically would inflict an inordinate degree of trauma, the different types of radiation shielding, to include Auger and higher dose rate radioisotopes used to isolate drug pipelines conducting substances at a dose rate than would be contained by the material of the pipeline itself.

Also addressed are the use of such a control system to alleviate the symptoms of urinary dysfunction such as urethra-noncompressive reinstatement of urinary continence, Vineberg-derived prevention of hypoxia and reperfusion in different contexts, such as venous stasis ulcers of the lower leg, targeted interdiction of a cirrhosis-inducing cascade, stereotactic drug steering by magnetic vectoring, discrete point, and point-to-point through-tissue, transmission, measurement, and telemetry, and targeted electrical and/or chemical autonomic motor assistance, in addition to numerous related topics.

Wireless body area networks with wireless transmission or telemetry is addressed with references provided in copending application Ser. No. 14/121,365, entitled Ductus Side-entry Jackets and Prosthetic Disorder Response Systems, filed on 25 Aug. 2014. The automatic drug selection and delivery control program or prescription data switches the drug reservoir catheters connected to each target ductus from among an unlimited number of drug supply reservoirs. In this, a body area network under simple or intelligent' complex, meaning hierarchical adaptive control, can be connected to transmit data through a wireless network.

### CONCEPT OF THE INVENTION

The invention relates first to a system for the segregated delivery of drugs to nidi or the locations of disease processes which eliminates several constraints that have limited drug efficacy in the past, and second to an automated system for determining the identity, apportionment, and dosing best suited to each morbidity and for the combination thereof and for effectuating the directly pipeline-targeted delivery of the drugs to the targets so that the treatment will have been optimized for each morbidity as well as the sum thereof. The two greatest impediments in the use of drugs are the risk of side effects and patient nonadherence to the prescription regimen.

Described here are fully, or closed-skin, implanted control systems that seek to overcome both of these disadvantages. Side effects arise when a drug introduced into the circulation comes into contact with tissue other than that aimed for. A familiar instance is the exposure to oncologic drugs which mitocidal, target the quickly replicating cells of a malignancy but also those which produce hair and gametes. Other examples include increased vulnerability to infection in patients prescribed immunosuppressives and the moon facies associated with steroids.

All drugs have side effects, and the medical benefits of limiting dosage levels to those which will not harm unintended organs, glands, or tissues while producing the optimal effect in that intended pertains to all drugs. Copending application Ser. No. 13/694835 addresses the targeting of radiopharmaceuticals not on the basis of an inherent metabolic affinity of the target organ such that of the thyroid gland for iodine, but rather through the application of magnetic attractive force to superparamagnetic such as magnetite or maghemite drug carrier nanoparticles to which the radiopharmaceutical is bound within a ferrofluid introduced into the pre- or post-heapatic systemic circulation rather than delivered directly to the target (see, for example, Wilfried Andrä, W. and Speer, T. 2010. Targeted Radionuclide Therapy, Lippincott Williams & Wilkins; Nowak, H. (eds.) 2006. Magnetism in Medicine: A Handbook, Hoboken, N.J.: John Wiley and Sons).

In copending application Ser. No. 16/873,914, entitled Vascular Valves and Servovalves—and Prosthetic Disorder Response Systems is described the use of such systems to administer surgical procedures, to include solid organ transplantation by means of a compound bypass procedure that switches the circulation of the recipient from his own native to the donor organ, and if appropriate, allows the donor organ to be harvested at the around the midway points in this switch, the donor organ then implanted as a second heart or a supernumerary thyroid gland, for example, where the native organ had grown sufficiently incompetent to justify placement of a coworker as an assist device.

Such a system can also administer an extracardiac reversal of a transposition of the great vessels, the semiautomatic removal and replacement with a prosthesis of any segment along a ductus in any length with its branches, to include the aorta or main pulmonary vessels, for example, in no case requiring a state of circulatory arrest. Some tissues have an intrinsic predilection for a particular agent moving through the circulation. The affinity for iodine of the thyroid gland makes it possible to chemically target the thyroid through the circulation without the need to literally isolate the iodine in a mechanical sense.

Following an organ transplant, the same system that administered the compound bypass transfer procedure will assure that immunosuppressives are released and optimally directed in perpetuity, thus eliminating deviation from the prescription regimen as a key deterrent in organ transplantation, especially in the very old, the very young, the very sick, the psychotic, and those with impaired cognitive ability. But the affinity of iodine for the thyroid gland is an exception.

Less familiar examples of drugs that treat the target while causing grave injury elsewhere in the body are anticholinergic and antimuscarinic negative inotropes used to subdue the excitable neurons in the detrusor muscle of which the hyperactivity causes overactive bladder and frequent urination. Little if at all problematic when used earlier in life, once the blood brain barrier begins to break down with age, these drugs progressively gain greater access to the neurons of the brain where they bring about and cooperate in bringing about dementia.

Another constraint in the systemic administration and dispersal of a drug is that the dose cannot be less than would affect the target tissue to a therapeutically meaningful extent but at the same time must not be so great as to adversely affect nontargeted tissue to an unacceptable extent, thus encouraging if not compelling the underdosing of the target, or intended, tissue while overdosing nontargeted, or unintended, tissue. In fundamental contrast to this limitation, directly pipe-targeting a drug to the target allows the dose, to which—absent a number of methods for eliminating it entirely—nontargeted tissue might be exposed to no more than a trace amount, to be freely set to that level optimal for the condition of the tissue when targeted.

Delivery thus means that drug selection and dosing need never make concessions to drug-drug or drug-nutrient interactions or the effect on nontargeted tissue. Unfortunately, in the treatment of cancer, the impetus to use the fewest drugs in the lowest dose must often be disregarded to save the patient (see, for example, Hahn, M. and Glatstein, E. 2005. “Principles of Radiation Therapy,” in Harrison's Principles of Internal Medicine, New York, N.Y.: McGraw-Hill, 16th Edition, pages 482-489). Another effect on pharmacy is the impetus to develop liquid drugs at high concentrations for automatically controlled microdrop drip release to allow minimization in the size of implanted drug reservoirs and thus avert the need for the patient to wear a power and pump pack.

A key factor in treatment with the aid of such means is that drug and electrical discharge delivery is targeted, that is, conveyed directly to the treatment site or sites, eliminating electrical or drug takeup and reaction by nontargeted tissues. The view reciprocal to that specified above is no less important. By allowing dosing that substantially omits nontargeted tissue, targeting considerably expands the utility of existing drugs, often in smaller doses, with fewer side effects, and at less expense. Moreover, the avoidance of a dependency upon intrinsic affinity combined with adverse side effects on nonaffiant tissue should not only optimize the efficacy of drugs long in use but expedite the approval of new drugs, fundamentally reducing the expense of pharmaceutical development.

Ductus side-entry connectors allow the secure connection of synthetic tubing to anatomical tubular structures, to include blood vessels and digestive conduits. An automatic ambulatory prosthetic disorder response system makes possible the continuous automatic monitoring, recording, and application of therapy that previously could be applied only while the patient remained confined to the clinic. The utility of state of the art ambulatory diagnostic tools, such as Holter monitors and event recorders (see, for example, The Merck Manual 18th edition, 2006, section 7, chapter 70, “Cardiovascular Tests and Procedures, page 597) in capturing diagnostic information without the need for continued vigilance in a hospital setting is thus generalized to the diagnosis of each in number of morbidities as well as the totality thereof.

Moreover, the therapy itself is availed of the fundamental benefits of direct pipeline delivery to the target, to include the ability to optimize dosages without exposure to nontargeted, often vulnerable tissue. The avoidance of adverse side effects, drug-drug, and drug-nutrient interactions is a fundamentally liberating consequence for drug development, application, and prescription. These interactions severely contstrain prescription to the point of hesitancy so that that the fewest drugs in the lowest are recommended, and in so doing, may result in the withholding of an additional drug or drugs or the use of these in greater concentration that would more contribute to patient relief.

To allow the drug may require reviewing the combination of drugs prescribed to determine on a drug-by-drug basis whether any of the others might be eliminated or substituted. Moreover, when evidence of an adverse interaction appears, serum concentration testing may become necessary to identify the source of the problem (see, for example, The Merck Manual 18th edition, 2006, pages 2516, 2517). In affording strict control over which drugs and nutrients may be allowed to come into contact, direct pipeline targeting eliminates all potential for the emergence of such hindrances.

Even in the clinic, to implement such a system requires stable and durable junctions between synthetic materials and native tissue. Ductus side-entry jackets and nonjacketing side-entry connectors must be long-lived, sufficiently stable in dimensions, reasonably conform to rather than encroach upon neighboring tissue, and minimally excite foreign body tissue reactions or otherwise cause discomfort. As a result, the ambulatory patient should be oblivious to the system.

That the limitation of drug dosing to less than optimal levels for no inherent reason, but rather because these cannot be isolated from other tissues during delivery to the target for fear of side effects has been overcome is quite likely to result in new levels of efficacy for many drugs that have long been known but never allowed to be delivered in the dose required to be most effective. If the disease is systemic and the nidus directly targeted, then a background dose of the drug or drugs is circulated. Similarly, the potential for metastasis of a carcinoma or sarcoma is suppressed with a systemic dose much reduced compared to that conventional, and possibly a regional dose somewhat higher in concentration, while the lesion itself is directly assailed with as concentrated an anticancer drug or drugs as will not provoke injury equally worrisome as the cancer itself.

The isolated delivery and targeting of relatively high dose rate antineoplastics or anticarcinogens is through radiation shielded passages and connectors, described and illustrated in copending applications Ser. No. 15/998,002 entitled Ductus Side-entry Jackets and Prosthetic Disorder Response Systems, and Ser. No. 14/998,495, entitled Nonjacketing Side-entry Connectors and Prosthetic Disorder Response Systems, both of which show radiation shielding devised to break down over time as well shielding that will not. Numerous issues omitted in this application, to include the various implementations of radiation and the use of superparamagnetic nanoparticle-carried drugs make it essential to review these applications.

For biomedical engineers and technically oriented internists, among others, the prerequisites to make possible the realization of a fully or almost fully implanted automated system to suppress a single chronic disease using negative feedback control would appear not simple but still clear enough to be dealt with on the basis of experience and intuition. Mostly what is needed is some technical background about automation, a good understanding of the problem and its interactions from the standpoint of internal medicine, an awareness of the availability of practical connectors, drivers, and analyte sensors, or detectors, that would be needed to institute the system so that it would continue to function over an indefinite but very long period.

However, even to create this relatively simple nonspecific monomorbid system, all of the essential components, described and illustrated in detail in the copending applications identified above, will usually not have been approved for implantation in humans and therefore remain unavailable, while references to various sensors which are available are cited in the literature. Here is taken up comorbid disease in which all of the components interact, making the maintaining of a running overall summary diagnosis to allow optimal treatment from the lowest level to that comprehensive not just complex but demanding of time not ordinarily granted to a single patient.

Here the comorbidies represent the upward arms or channels of progressively more highly integrated and coordinated data. A fully implanted hierarchical control system of the kind indicated comprises three major components:

1. A fully implanted set of sensors to generate ground level diagnostic data specific to each component morbidity. Two morbidities may require more than just one or two sensors each, and an increase in the number of sensor can determine the number of nodes, or subcontrollers, at higher levels into which they feed.

2. The lower level nodes coordinate the ground level diagnostic sensor output data sent to them from the ground level. The larger the number of sensors, the greater is the probability that more than one node at the next or two next higher levels will intervene between the ground level and the master controller. Moving up the hierarchy, the degree of data integration becomes greater and the number of nodes fewer. First, the data for each morbidity is integrated, then the data for the other morbidity or morbidities must be coordinated with this data. At the penultimate level in the hierarchy, the data from the component morbidities is integrated and passed up to the master controller for remedial action.

Based upon the prescription-program, the master controller, which can identify problems at any subordinate level, evaluates the summary data covering the combination of morbidities, compares the values across the morbidities to the those in the normal range, and passes the adjustments to be effected down through the levels as ‘motor’ commands to effect the adjustment. More specifically, the master controller translates its comprehensive data into the commands issued to the system drug reservoir outlet motors—or if delivered by gravity feed, opens the stopcock of the reservoir, or energizes and monitors the electrical end-effectors assigned—and monitors each. When the preferred end-values cannot be specified, the master node, or master controller, can be programmed to apply a sequence of test doses at different levels to find that which yields the best overall result.

3 The third major component is the hardware, the pipeline distribution system consisting of a set of catheteric blood and drug pipelines to deliver the drugs and/or other agents injected at a body surface port such as those shown in copending application Ser. No. 15/998,002, entitled Ductus Side-entry Jackets, FIGS. 27 and 28 and copending application Ser. No. 16/873,914 entitled Vascular Valves and Servovalves—and Prosthetic Disorder Response Systems, FIGS. 26A thru 26C into the drug reservoirs to pass through the ductus fluid lines leading to the side-entry and nonjacketing connectors and electrical end-effectors described in the copending applications cited and through these into the target nidi, tissues, or their blood supply. The entry holes respective of each pipeline can also serve to allow an endoscope to be passed down to the treatment site.

Through this distribution system, the control system effectuates drug and nondrug, such as electrostimulatory or neuromodulatory therapy that is directly targeted by transmission through a fluid pipe or electrical line. Extraordinarily, commands can be transmitted by radio, generally ‘Bluetooth’. Total body networks, transcutaneous, or transdermal, enegy transfer, remote diagnostics, and medical telemetry are covered.

If it poses a potential problem, any trace residue, even radioactive, not taken up within the parenchyma, endothelium, or urothelium and as a result continues to pass into the venous drainage is easily eliminated through a number of methods described in the foregoing applications. The incorporation of accessory channels allows all pipelines and end-connectors to be intermittently drip-fed or occasionally flushed through with clot, crystal, and/or biofilm buildup solvent as well as allow the intermittent addition to the primary drug of an adjuvant, for example In this regard, accessory channels eliminate a major drawback in the usability of polymeric tubing to serve as small shunts and bypasses.

The first of these was described in detail in the copending applications cited, which not only clarify the structure of the different type end connectors used but the applications of these over a broad spectrum of internal medicine. he delivery system must incorporate means for connection to native tissue that should never require followup maintenance that cannot be accomplished with the accessory channels provided. In the copending applications specified, system jacketing and nonjacketing connectors, the different types and the numerous applications of each type have been described and illustrated in detail.

In the treatment of monomorbid disease, such as diabetes or heart failure, the system master controller is a microcontroller. Where both of these conditions, elevated blood pressure, and atheroclerosis combine in metabolic disease—in light of its prevalence, an ideal example—a more capable controller, a microprocessor, programmed at a lower level to address each disease component and at a higher level to cross-level treatment among the component morbidities to achieve the best overall homeostatic condition is used. The implication that the system maintains a running record of drug release/outcome relations which can be printed out as essential for diagnostics and the formulation of updated or new prescription-programs is correct.

By now, the concept of hierarchical control is over a half century old, and appears to have been limited in application to the programming of autonomous industrial robots and ‘rovers’ for deployment on other planets (Mesarovic, M. D., Marco, D., and Tashahara, Y. 1970. Theory of Hierarchical Multilevel Systems, Academic Press: New York, N.Y.), and the resolution of performance optimization issues in production plants. Similarly to the industrial applications of hierarchical control where the object was to progressively adjust an unfolding or building process as would best approximate the specified end result, in medicine, the result is not designed but given by nature as a condition of normalcy. There appears to be no evidence that the concept of hierarchical control was ever contemplated as a powerful tool to coordinate the remedial action to be taken on the basis of often complex data which is the ordinary situation in internal medicine.

Even though hierarchical control has been around for decades, without means for safely, durably, and connecting to native ductus along the vascular tree through a secure and leak-free junction with catheteric drug and blood pipelines rendered invulnerable to the accumulation of clot, crystal, and/or biofilm, the relation, indeed realization of the possibility to apply hierarchical networked feedback to automatic drug delivery has remained unfeasible. Sensors can be collocated with the means that make possible the targeted release of drugs to the location respective of each.

For this reason, immediate and automatic remedial drug delivery, not just information as to the status of the patient, can be achieved. As will become clear, the strategically located sensors, ductus side-entry connection pump-pairs and jacket sets to be described make possible the targeted delivery of drugs through automatic response that is immediate. Were the condition to exceed the range of adjustment for which the system had been set, the exigent readings can be transmitted to a clinician able to adjust the dosing by remote control.

Every tissue in the body is either part of and therefore directly, or supplied by and therefore indirectly, accessible through vessels and/or ducts. There is no disease in which vascular and other supply and drainage lines are uninvolved and signal the local dysfunction to higher control centers. Symptoms even appear in bodily systems that would seem qualitatively unlike and remote from that of origin. Regional enteritides can induce arthritis. Osteoporosis and Paget's disease of bone (osteitis deformans), for example, are disorders often secondary to endocrine disease that affect the skeleton.

If arterial applications are stressed, it is because of the disproportional involvement of vessels in death from disease. No bodily conduit, to include the smallest, is analogous to inert plumbing; all are integrated into a hierarchy of negative feedback loops from the brain down to the individual cells to actively interact with the passing contents (see, for example, Jameson, J. L. 2005. “Principles of Endocrinology,” in Harrison's Principles of Internal Medicine, New York, N.Y.: McGraw-Hill, page 2072).

Every bodily conduit communicates directly or indirectly with all the tissues in the body—not just by transmitting luminal contents, but by signaling local function to higher control centers. In endothelial function, for example, the linings of blood and lymphatic vessels actively secrete vasodilators such as relaxing factor (like indistringuishable from nitric oxide), bradykinin, potassium ions, and adenosine and vasopressors or vasoconstrictors such as endothelins, epinephrine, norepinephrine, dopamine, thromboxane, and insulin, all tied into coordinated feedback loops, which continuously adjust the degree of contraction, hence, the blood pressure.

That vessel wall, segment, and organ drug targeting has not progressed beyond the drug eluting stent is due to an inadequacy of methods and means for limiting drug delivery to the site that requires treatment and would allow different drugs to be delivered in doses not limited by intolerances to tissues beyond the target area. Whether access through ‘keyhole’ incisions at the body surface is more invasive than transluminal access may not be true.

In addition to communication affected by the autonomic nervous system, the luminal wall can release signaling proteins, such as chemokines and interleukins, and the luminal contents can include enzymes, hormones, cells containing cytokine signaling proteins, and so on, so that remote tissues are affected as well. As a result, there is no disease in which bodily conduits are uninvolved. No bodily conduit is analogous to inert plumbing; all are integrated into a hierarchy of negative feedback loops from the individual cells to the brain to actively and appropriately interact with the constitution, pressure, and velocity of passing contents.

Collaterally, vasopressin, or antidiuretic hormone, produced in the hypothalamus and released by the pituitary gland in response to a decrease in blood volume exerts a pressor effect and acts as a diuretic by reducing the volume of urine, thereby conserving the volume of blood. That the caliber of blood vessels, for example, is adaptive locally as well as systemically demonstrates that control is effected by a hierarchy of control loops wherein those subordinate interact with those progressively more encompassing until the center just above the brainstem is reached. Central mechanisms initiate the release of circulating vasoconstrictors or vasodilators that cause the linings of blood vessels to contract or relax in response to the condition of the circulatory system, which includes cardiac output, partial pressures of oxygen and carbon dioxide, and the existing concentration of hormones and electrolytes in the blood.

Blood pressure as the product of cardiac output and peripheral resistance subsumes numerous interrelated contributory closed loop actions responsive to emotional state, level of exertion, temperature, metabolism as affected by ingesta, disease, medication, and gas exchange in the lungs effected by cellular level feedback between every cell and its immediate environment. Maintaining normal function in the walls of bodily conduits is thus central to and inseparable from maintaining normal function. Much the same hierarchical integration mutuates between the wider physiological context and any other bodily conduit, whether ureter, gamete transporting duct, the airway, choroid plexus and arachnoid villi, or lymphatic vessel.

While the body is able to compensate for numerous forms of degradation, such as those associated with aging, a failure to produce an essential enzyme or to produce an essential protein as the result of a genetic defect or progressively emerging alteration, for example, is sufficiently anomalous that the body lacks sufficient responsive measures. Thus, up to the degree of deviation that can be accommodated, an atherosclerosed artery is continuously remodeled, preserving its luminal diameter, for example, but the inadequate synthesis of insulin, resulting in diabetes, or tyrosine, resulting in phenylketonuria, for example, demand human intervention.

Such anomalous defects, to which the body has limited if any adaptive or accommodative compensatory response, account for much of internal medical practice. Application to controlled steering of a prosthetic hand has been addressed (Light, C. M., Chappell, P. H., Hudgins, B., and Engelhart, K. 2002. “Intelligent Multifunction Myoelectric Control of Hand Prostheses,” Journal ofMedical Engineering and Technology 26(4):139-146; Chappell, P. H. and Kyberd, P. J. 1991. “Prehensile Control of a Hand Prosthesis by a Microcontroller,” Journal of Biomedical Engineering 13(5):363-369), but nowhere does there appear the application of hierarchical control to continuous adjustment in the execution of a prescription or any end motion-unrelated medical use.

A system for the delivery of drugs under the control of a hierarchical control system is analogous to the kind of system used to control a remote vehicle, for example. Such a system, where data is collected as to the best overall outcome across a plurality of morbidities treated with various combinations of drugs administered is analogous to the kind used to control a remote vehicle over uneven terrain, where data concerning the contour of the ground covered and the orientative response thereto is continuously collected for use to adapt for and optimize continued level transit, for example.

While not as applied to internal medicine, hierarchical control, whereby sensors send input data up through levels of more encompassing data integration and commands usually proceed in reverse down through the same chain, is a well developed field (see, for example, Raisch, J., Schmuck, A.-K., Gromov, D., and Geist, S. 2021. “Hierarchical Control Theory,” online at https://www.mpi-magdeburg.mpg.de/95036/Hierarchical-Control-Theory; Dellaert, F. 2020. “Hierarchical Control,” online at https://www.cc.gatech.edu/˜dellaert/07F-Robotics/Schedule_files/02-Hierarchical Control.ppt.pdf; Merel, J., Botvinick, M. and Wayne, G. 2019. “Hierarchical Motor Control in Mammals and Machines,” Nature Communications 10:5489, online at https://doi.org/10.1038/s41467-019-13239-6; Schlenoff, C., Albus, J., Messina, E., Barbera, A. J., Madhavan, R., and Balakirsky, S. 2006. “Using 4D/RCS to Address AI Knowledge Integration,” Artificial Intelligence Magazine 27(2):71-81; Aguilar, J., Cerrada, M., Mousalli, G., Rivas, F., and Hidrobo, F. 2005. “A Multiagent Model for Intelligent Distributed Control Systems,” 191-197; http://www.mpi-magdeburg.mpg. de/95036/Hierarchical-Control-Theory; Meystel, A. M. and Albus, J. S. 2002. Intelligent Systems, New York, N.Y.: John Wiley and Sons; Albus, J. S. 2000. “4-D/RCS [Four Dimensional Real-time Control System] Reference Model Architecture for Unmanned Ground Vehicles,” in Proceedings of the 2000 IEEE International Conference on Robotics and Automation, New York, N.Y.: Institute of Electrical and Electronics Engineers; volume 4, datalogue number 00CH37065, pages 3260-3265; Takahashi, Y. and Asada, M. 1999. “Behavior Acquisition by Multi-layered Reinforcement Learning,” in Proceedings of the 1999 IEEE International Conference on Systems, Man, and Cybernetics, New York, N.Y.: Institute of Electrical and Electronics Engineers; pages 716-721; Albus, J. S. 1996. “The Engineering of Mind”. From Animals to Animats 4,” in (Maes, P., Mataric, M. J., Meyer, J.-A., Pollack, J., and Wilson, S. W. (eds.) Proceedings of the Fourth International Conference on Simulation of Adaptive Behavior (Complex Adaptive Systems): Cambridge, Mass.: MIT Press; Albus, J. S. and Meystel, A. M. 1996. “A Reference Model Architecture for Design and Implementation of Intelligent Control in Large and Complex Systems,” International Journal of Intelligent Control and Systems 1(1):15-30; Albus, J. S. 1995. “RCS: A Reference Model Architecture for Intelligent Systems, Association for the Advancement of Artificial Intelligence Technical Report SS-95-02, available at http://aaaipress.org/Papers/Symposia/Spring/1995/SS-95-02/SS95-02-001.pdf; Albus, J. S. 1993. “A Reference Model Architecture for Intelligent Systems Design,” Chapter 2, pages 27-56 in Antsaklis, P. J. and Passino, K. M., eds., An Introduction to Intelligent and Autonomous Control, Baltimore, Md.: Wolters Kluwer Academic Publishers; Hayes-roth, F., Erman, L., and Terry, A. 1992. “Distributed Intelligent Control and Management (DICAM) Applications and Support for Semi-automated Development,” in Keller, R/M. (ed.), Working Notes from the 1992 AAAI [Association for the Advancement of Artificial Intelligence] Workshop on Automating Software Design, National Aeronautics and Space Administration Technical Reports Server Document ID 19930008310; Jones, A. T. and McLean, C. R. 1986. “A Proposed Hierarchical Control Model for Automated Manufacturing Systems,” Journal of Manufacturing Systems 5 (1): 15-25; Findeisen, W. 1984. “The Essentials of Hierarchical Control,” in Thoft-Christensen, P. (ed.), System Modelling and Optimization. Lecture Notes in Control and Information Sciences 59:38-61; Findeisen, W.; Bailey, F. N., Brdys, M., Malinowski, K., Tatjewoki, P., and Wozniak, A. 1980. Control and Coordination in Hierarchical Systems, Chichester, England/New York, N.Y.: John Wiley and Sons, Issue 9 of the International Series on Applied Systems Analysis, Wiley-Interscience; additional references provided below).

Control therefore is preferably of sensor response adaptive closed loop control over the delivery of each drug in the turret, control of the pump and turret stepper motors under open loop controlled. While the same degree of complexity and expense is not warranted in less serious cases, in a patient with an unstable life-threatening condition, adaptive response justifies the implantation of sensors tied into closed loops in a wireless body area network with automatic adaptive response in the dosing of each drug by means of a hard real time adaptive hierarchical, or nested, complex control system.

Provided with proper sensors properly located, such a system can be programmed to assimilate or ‘learn’ events as these are experienced, such as the action of a drug at an interval other than expected (Albus, J., Bostelman, R., Hong, T., Chang, T., Shackleford, W., and Shneier, M. 2006. “The LAGR [Learning Applied to Ground Robots] Project: Integrating Learning into the 4D/RCS [4 Dimensional Remote Control System] Control Hierarchy,” International Conference in Control, Automation and Robotics—ICINCO 06, Setubal, Portugal, available at http://www.nist.gov/customcf/get_pdf.cfm?pub_id=822702). Unless interrupted by an adverse event, the drug regimen continues unaffected. In such a hierarchical control system, the processors of a monolithic integrated circuit or microchip multicore microcontroller are partitioned to support one control node each, that programmed to function at the highest level of control as the master node sent the inputs from and having a time horizon comprehensive of the subordinate nodes, of which each contributes inputs to the pumps and jackets of the set based upon the sensors that feed it.

Meaning of an Automatic Adaptive/predictive Ambulatory Prosthetic Disorder Response System

System desiderata and capabilities are addressed in different contexts and therefore appropriately addressed in different sections below such as that entitled Local and Systemic Implications of Automatic Sensor-driven Targeted Drug Delivery. With a portable (wearable, ambulatory) prosthetic disorder response system, the clinician specifies the target ductus, the drugs to be delivered to each, the dose regimen, and any additional factors pertinent thereto. According to the present concept, a pharmacist-programmer enters this into a program whereby each drug is provided in response to the conditions sensed. To deliver drugs automatically and adjust the dosing, the prescription, or adaptive drug delivery program, responds to diagnostic sensor feedback under the control of a medically adapted hierarchical (nodal, nested-levels) ‘intelligent’ hard real-time ‘pathfinding’ control system.

Depending upon the intricacy and frequency of differential control required of either pump in the pump and jacket set, each node controls either one of the pumps or the modular plug-in pump-pair as a subsystem in the pump-pack, usually cinched about the waist. While sensors, fluid lines, and connectors must be implanted, the control circuitry, power source, and pumps need not. Generally, the latter are implanted only when the condition or conditions treated are expected to persist to the end of life. In the case of progressive disease, the sensor-driven automatic drug delivery system spontaneously adjusts the intervals and dose of drugs in accordance with the prescription-program,

Representation in the drawing figures of system componentry as housed in a body surface-worn, or paracorporeal, pump, power, and/or control pack pertain no less to system implantation where these parts are much miniaturized to allow full, or closed-skin, implantation. To the extent practical, where comorbid conditions must be treated, each such component disease is assigned to a respective node and modular plug-in pump-pair and jacket set, and the master microprocessor programmed to coordinate the delivery of drugs among the nodes.

System Control of Multidrug Delivery System

Control therefore is preferably of sensor response adaptive closed loop control over the delivery of each drug in the turret, control of the pump and turret stepper motors under open loop controlled. While the same degree of complexity and expense is not warranted in less serious cases, in a patient with an unstable life-threatening condition, adaptive response justifies the implantation of sensors tied into closed loops in a wireless body area network with automatic adaptive response in the dosing of each drug by means of a hard real time adaptive hierarchical or nested complex control system.

Such a system, where data is collected as to the best overall outcome across a plurality of morbidities treated with various combinations of drugs administered is analogous to the kind used to control a remote vehicle over uneven terrain, where data concerning the contour of the ground covered and the orientative response thereto is continuously collected for use to adapt for and optimize continued level transit, for example (reference provided above and additional references cited below).

That is, with the proper sensors, such a system can be programmed to assimilate or ‘learn’ events as these are experienced, such as the action of a drug at an interval other than expected (Albus, J., Bostelman, R., Hong, T., Chang, T., Shackleford, W., and Shneier, M. 2006. “The LAGR [Learning Applied to Ground Robots] Project: Integrating Learning into the 4D/RCS [4 Dimensional Remote Control System] Control Hierarchy,” International Conference in Control, Automation and Robotics—ICINCO 06, Setubal, Portugal, available at http://www.nist.gov/customcf/get_pdf.cfm?pub_id=822702).

Unless interrupted by an adverse event, the drug regimen continues unaffected. In such a hierarchical control system, the processors of a monolithic integrated circuit or microchip multicore microcontroller are partitioned to support one control node each, that programmed to function at the highest level of control as the master node sent the inputs from and having a time horizon comprehensive of the subordinate nodes, of which each contributes inputs to the pumps and jackets of the set based upon the sensors that feed it.

In more advanced use—appropriate for the treatment of more complex comorbid disease where a certain apportionment of drugs among the treatment sites achieves the best overall consequence—the dispensing of drugs is automated, necessitating the implantation of additional components. Each component morbidity is assigned a channel or arm of control in a hierarchical control system which takes biosensor inputs at the lowest level. The inputs are passed for coordination to a control node, an intermediate chip-microcontroller, thence to a next higher node which coordinates treatment with its counterpart or counterparts in the other control channel or channels, the number thereof and need for coordination among these dependent upon the number of distinguishable morbidities.

These nodes then pass their outputs to a master node microprocessor. By analogy with the nervous system, such can be characterized as the system sensory function. Overall coordination of drug delivery (or other therapy, such as the application of heat), analogous to motor function, is then governed by the master node, a microprocessor which coordinates the inputes from the nodes next lower in rank and passes control signals down the channel in a motor sense, causing implanted drug reservoir outlet valves or pumps to release the drugs as directed.

When the condition of the patient allows, fitting of the system is preceded by an initial test period similar to that used in placing an elecrostimulatory neuromodulator except that the question is not whether to implant the device but rather what combination drugs would best be used. The implanted system is used to test different drugs, the best overall result with minimal drug interaction thereby made discernible. This determined, a pharmacist-programmer prepares a prescription-program for execution by the master node. Immediately lesion- or nidus-targeted and kept from the general circulation, medication delivered thus is substantially more effective in smaller doses and spares nontargeted tissue.

While in use independently, the local control module, itself a chip microcontroller, and associated components nevertheless represent a single node of the overall prosthetic disorder response system governed by a a central microprocessor as the master control node. The later addition of another system module, such as requires a pump-pack and fluid lines to deliver synthetic mucus and digestive enzymes, then requires the activation of another node.

If the implanted or local control module is so capable, it continues to support the digestive module previously implanted, and has the new node added. When the digestive function need not be coordinated with the added function, it is most expeditious to allow the existing implant to continue to function independently. Otherwise, it is equally expeditious to place the previously implanted local control module as a node under the control of an added microcontroller in the pump-pack, or if the local controller is so capable, assign to it overall control. Later access governs the positioning of components.

Automatic disorder response system design strives for freedom from the impediment of a belt-worn power, control, and/or drug reservoir and outlet motor pack, conceding to the use thereof only when necessary. Implementation is preferably in the form of a fully, or closed-skin, system. Nevertheless, for pictorial clarity in copending applications pertaining to ductus side-entry jackets and nonjacketing side-entry connectors, system components are depicted in mechanical form.

The need for a paracorporeal pack arises when the number of system components exceeds that implantable without obtrusion into neighboring tissue to cause discomfort. Currently, conditions involving fewer morbidies can be accommodated by a fully implanted system. Telemetric data transmission, a total body network, and the replacement of hard wires with short distance radio transmission can be obtained in small shape factor forms. Once transcutaneous power transfer allows the use of small antennae, this too can be implanted.

The relegation of nonimplanted components to an externally worn, or paracorporeal, body pack can include the hierarchical master node microprocessor, subordinate level node microcontrollers along each morbidity channel sent to the microprocessor, the power source, and external drug reservoirs or storage canisters, and pumps. Clearly, in the treatment of disease with the potential to result in death if not treated, a definite preference favors a fully implanted system, various means for fitting components inside the body such as tissue extension addressed in the copending applications cited above as well as herein.

Ambulatory Adaptive Prosthetic Disorder Response System Control

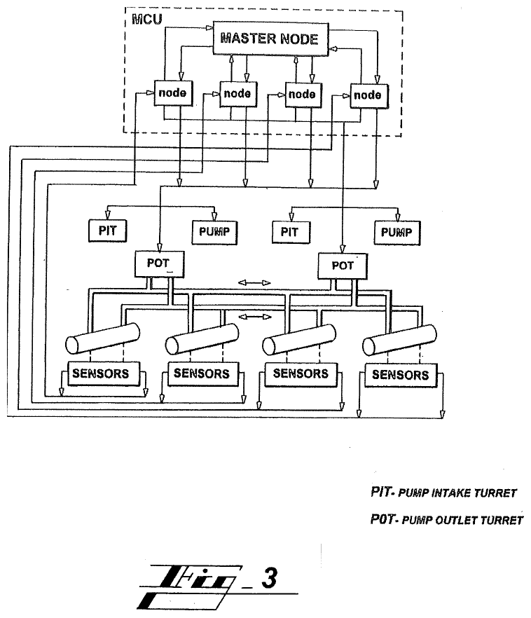

FIG. 2 provides a schematic of the control hierarchy for a single pump-pair in support of four jackets in the pump-pair and jacket set, the control program, that is, the prescription-program, of the master node, a microprocessor, determined by the specific or comorbid conditions to be treated. Nodes subordinate to the master node are generally microcontrollers. FIGS. 2 and 3 provide a schematic of the pump-pack, jacket set, and control system.

Unlike FIG. 4, in FIG. 3, only the control train is represented, the distinction between intra and extracorporeal elements omitted. An extracorporeal pack affords considerably more space and can hold a larger volume of numerous drugs, other therapeutic agents, and equipment maintenance solutions. While shown as carried in a body pack, the control hierarchy is implantable with the impediment of a pack eliminated.

When implanted, the contents labelled body pack at the lower left in FIG. 4 are miniaturized; otherwise, FIG. 3 applies no less to a fully implanted as to a body pack carry system. Also when implanted, to preclude complications due to encroachment upon or strangulation of tissue by wires, data intercommunication from the sensors and subordinate nodes and control signals from the master node are preferably by wireless, or Bluetooth transmission. For pictorial clarity, where the electrical and fluid lines between nodes and jackets are actually separate and distinct, those between nodes and jackets are shown as consolidated until finally led to each jacket, and remote sensors and auxiliary drug supply reservoirs have been omitted.

Electrical connectors, more remote sensors, drug supply reservoirs and outlet pumps controlled by the master node have been omitted. If provided with the requisite switching and valving, the fluid and electrical lines shown as shared could support each jacket independently but not simultaneously, the utility thereof contingent upon the condition or conditions to be treated; simultaneous capability is accomplished by furnishing the components necessary. Hard-wired intracorporeal electrical connections that would pose the risk of strangulating a structure or tissue intervening along the delivery route are preferably implemented by means of short range radio transmission such as provided by ‘Bluetooth. ’

Such lines of communication can serve to interconnect local implanted sensors, their co-level nodes, intermediate nodes, and the hierarchical control master node, a microcontroller in uncomplicated or monomorbid, a microprocessor in comorbid disease. In FIGS. X and X, single lines are electrical, or if it is found difficult to route the electrical lines without the risk of strangulating intervening structures, then connected by wireless Bluetooth transmission rendered selective by difference in carrier frequency.

If virtually simultaneous operation cannot be achieved with a single carrier transmitter switching among the jackets, the microprocessor is provided with more than one transmitter. The addition of a module is coordinated with the module or modules already inserted; however, because when targeted to specific tissue, most if not all drugs are kept separate, the regimen overall as administered by the master controller over the nodes usually need not effect significant adjustments among these to accommodate the addition or removal of a power, control, and pump pack if such is needed.

While almost all practical applications will have been entirely translated into fully implanted miniaturized electronic versions of the equivalent mechanical components depicted in the drawing figures, by extracorporealizing numerous components, a pack does offer the benefits in the elderly and very young of both sparing the internal weight of these components (turreted pumps, batteries, possibly nodes) as well as freeing the application of changes in componentry from the need to reenter, even endoscopically.

Here a more literal mechanical representation of components which would almost always be implanted in miniaturized electronic forms equivalent to those mechanical that might be relegated to a belt-worn pack are primarily for pictorial clarity. Unless a practical power, control, and pump pack is provided with batteries which can be recharged through a small socket or transcutaneous-type energy transfer, a separate openable compartment can be provided to house disposable batteries. To prevent tinkering by a curious child, for example, the power, control, and pump pack can be locked, the key or keys maintained in the clinic.

A prescription-program can be executed by a multicore microcontroller of which each core or cog is programmed as a time division multiplexed node in the control hierarchy. Where magnetically susceptible carriers with or without a carried extractate (extractant) will be so small in volume as not to require removal, high energy product permanent magnets, ordinarily made of neodymium iron boron, are preferred. The addition of a module is coordinated with the module or modules already inserted; however, because when targeted to specific tissue, most if not all drugs are kept separate, the regimen overall as administered by the master controller over the nodes usually need not effect significant adjustments among these to accommodate the addition or removal of a pump-pack.

Such a prescription-program can be executed by a multicore microcontroller of which each core or cog is programmed as a time division multiplexed node in the control hierarchy. Where magnetically susceptible carriers with or without a carried extractate (extractant) will be so small in volume as not to require removal, high energy product permanent magnets, ordinarily made of neodymium iron boron, are preferred.

Where extractate debris or detritus will be slight, a permanent magnet jacket that detains the debris has a side grating that allows the debris to be extracted with the aid of a powerful extracorporeal electromagnet. Generally, the debris if at all toxic will be equally so in the tissue surrounding the ductus; however, when extracted, it can be dispersed so as to reduce the immediate burden or concentration to a tolerable level. If the extractate debris is more toxic or radioactive, then electromagnetic extraction jackets such as shown in FIGS. 13 thru 15 and described below remove the extractate entirely from the body. The consecutive jackets along the ductus in FIG. 15, ordinarily a vessel, are connected by a flush-line from a supply to a separate waste reservoir in the pump-pack, washing the pole of each electromagnet 75 where the debris accumulates along the way.

Instead of ambulatory means for continuously, automatically, immediately and autonomously responding to the condition sensed, readings are transmitted to a specialist for review and the writing of a prescription. The delay in this process is a conspicuous deficiency. Usually, the overall sequence in which the drugs are delivered to each side-entry jacket is maintained whether the jackets are placed along a single ductus, or where interrelated and interdependent organ systems are affected, along ductus belonging to different organ systems. In more complex situations, nested levels of program control, or nodes, each supporting a jacket incorporating symptom and remedial substance delivery and level-measuring sensors, are used.

Control System Options

The nodes can consist of time division multiplexed cores of a multicore microcontroller (see, for example, Schoeberl, M., Brandner, F., Sparsø, J., and Kasapaki, E. 2012. “A Statically Scheduled Time-division-multiplexed Network-on-chip for Real-time Systems,” pages 152-160, Networks on Chip (NoCS), 2012 Sixth Institute of Electrical and Electronics Engineers/ACM International Symposium on, Lyngby, Denmark, available at http://www.jopdesign.com/doc/s4noc.pdf; Sparso, J. 2012. “Design of Networks-on-chip for Real-time Multi-processor Systems-on-chip,” in 12th International Conference on Application of Concurrency to System Design, Hamburg, Germany pages 1-5; Paukovits, C. and Kopetz, H. 2008. “Concepts of Switching in the Time-triggered Network-on-chip,” in Proceedings of the 14th Institute of Electrical and Electronics Engineers International Conference on Embedded and Real-Time Computing Systems and Applications (RTCSA '08), Kaohsiung City, Taiwan, Republic of China, pages 120-129; Schoeberl, M. 2007. “A Time-triggered Network-on-chip,” in International Conference on Field-Programmable Logic and its Applications (FPL 2007), pages 377-382; Kopetz, H. and Bauer, G. 2003. “The Time-triggered Architecture,” Proceedings of the Institute of Electrical and Electronics Engineers, 91(1):112-126; Wiklund, D. and Liu, D. 2003. “SoCBUS: Switched Network on Chip for Hard Real Time Embedded Systems,” in Proceedings of the 17th International Symposium on Parallel and Distributed Processing (IPDPS'03), Los Alamitos, Calif., Institute of Electrical and Electronics Engineers Computer Society, page 78a), which communicate with the higher node or core programmed to function as master or ‘supreme’ node or controller, and if pertinent, directly with one another.

The distribution of control between the brain and subordinate circuits and ganglions a salient feature of the nervous system, such a hierarchical scheme may be seen as analogous to the relation between the motor cortex and subsidiary or more localized control circuits in the spinal cord, for example. Here such a control tree receives feedback from the sensors associated with each jacket to continuously adjust and coordinate the dosing of the drug delivery program in detail, and overall.

Subordinate or ‘intimal’ nodes closest to their respective sensors feed into a channel of control directed to one morbidity among morbidities, an organ, organ system, lesion, or midus, the sensor readings used by the master node to apportion the actuation of nondrug therapeutic components such as electrostimulators and the release among these targets of the fewest drugs in the smallest doses as is best likely to reinstate homeostasis or overall health to the extent possible.

The automatic adaptive response hierarchical control system consists of local microcontrollers—usually positioned within the ductus side-entry and impasse jackets and nonjacketing side-entry connectors which represent the subordinate hierarchical levels assigned to the morbidities. These take sensor inputs, which to minimize dissection and achieve the maximum compactness, usually incorporated into the local jacket or connector—and coordinate these within their respective subsystem, or channel of morbidity control, typically assigned to an organ- or organ system-defined channel of morbidity depicted in FIGS. 2 and 3.

Each morbidity is assigned a channel or arm of control in a hierarchical control system which takes sensor inputs at the lowest level. The inputs are passed for coordination to a control node, an intermediate chip-microcontroller, thence to a next higher node which coordinates treatment with its counterpart or counterparts in the other control channel or channels, the number thereof and need for coordination among these dependent upon the number of distinguishable morbidities.

As to route of administration, drugs are infused, that is, released directly into the vascular tree, usually into the supply artery of the organ or tissue volume targeted. In advanced and more complicated use demanding the treatment of complex comorbid disease where the dose of each drug to be delivered to each of numerous target sites must be timed to take into account the time to onset of each drug as well as the exact dose required within the context of the sum of drugs to bring about that combination of drugs most likely to optimally curb the disease overall.

The ‘inertia’ and delay in affecting some physiological parameters considerably greater than it is for others, depending upon the application, no individual or composite form of control, to include model predictive, fuzzy, and proportional-integral-derivative can be ruled out. In general, using different controllers in each type implanted drug reservoir outlet pump or if necessary, belt-worn power, control, and pack is more costly than is the use of a standard microcontroller and development environment; nevertheless, provided simple applications and embodiments prevail for a given type pump-pack, the smaller cost of a simple or hobby grade controller is preferable.

Hierarchical control has been available for decades; however, with no means for safely converging with ductus through a secure junction, the relation of hierarchical networked feedback to automatic drug delivery has remained elusive. Because the sensors are associated with collocated means for the targeting of drugs to the location respective of each, immediate and automatic remedial drug delivery, not just information as to the status of the patient, are obtained.

The penultimate nodes in the hierarchy pass their outputs to a master node microprocessor. By analogy with the nervous system, such can be characterized as the system sensory function. Overall coordination of drug delivery (or other therapy, such as the application of heat), analogous to motor function, is then governed by the master node, a microprocessor which coordinates the inputs from the nodes next lower in rank and passes control signals down the channel in a motor sense, causing implanted drug reservoir outlet valves or pumps to release the drugs as directed.

The overall consequence of the combination of drugs released or other therapy applied such as electrostimulatory or thermal entered into memory, the system ‘learns’ the best combination at a given time and adapts to changes with time, this pattern having diagnostic and prognostic value. Moreover, if automated, the system is able to edit its own prescription-program originally prepared by the pharmacist programmer and thus maintain the optimal time-adjusted therapeutic response to treat the component morbidities or leasions.

A ductus or impasse side-entry jacket equipped with the required electromagnets will draw any sufficiently magnetic field susceptible particle-bound drug outward and through the surrounding lumen wall. The position of the side-entry jacket thus targets the level along the ductus, and the magnetic force targets the intramural lesion in that segment. A lesion such as an atheroma is therefore ‘washed over’ and penetrated by the drug, which can be released continuously or at intervals throughout the day.

Local and Systemic Implications of Automatic Sensor-driven Targeted Drug and Electrostimulatory Delivery

Symptoms even appear in bodily systems that would seem qualitatively unlike and remote from that of origin. Regional enteritides can induce arthritis. Osteoporosis and Paget's disease of bone (osteitis deformans), for example, are disorders often secondary to endocrine disease that affect the skeleton. If arterial applications are stressed, it is because of the disproportional involvement of vessels in death from disease. Bodily conduits are not analogous to inert plumbing; in fundamental contrast to synthetic tubing, the walls of vessels, lymphatics, hormonal ducts, and gut are integrated into a hierarchy of negative feedback loops that to adaptively interact with the passing contents, extend from individual cells to the brain (see, for example, Jameson, J. L. 2005. “Principles of Endocrinology,” in Harrison's Principles of Internal Medicine, New York, N.Y.: McGraw-Hill, page 2072).

When a bodily conduit is itself diseased, effective and efficient treatment requires that medication be actively drawn into, not merely pass through the line. Allowing the medication to pass lesions wastes medication that if targeted would have contributed to an effective dose, exposes healthy tissue downstream to the wasted dose, results in complications, and increasing the dose to increase absorption only increases the waste and the risk. When the supply zone or territory or downstream segment becomes diseased, the contents passing through the line should be adjusted or supplemented to promote healing. While therapeutic agents are often best restricted to frankly diseased tissue, the pathways in which the affected tissue participates, and therewith, the far-reaching relations of that tissue to other tissue, means that the propagation of disease from that tissue to other tissue is not so restricted.

For example, the central negative feedback loops that govern angiotension flow along the hypothalamic-pituitary-adrenal axis. The central loops incorporate, integrate, and drive subsidiary loops more and more local in level down to the individual cells. Tied into the neuroendocrine and autonomic nervous systems, the hypothalamic-pituitary-adrenal axis responds to systemic blood volume by continuously regulating the blood serum levels of steroid hormones produced in the adrenal cortex, such as cortisol, and in the kidney, such as angiotensin II. Angiotensin II directly effects vasoconstriction and secondarily effects the release of aldosterone to regulate the balance between sodium and potassium in the blood, thus enlisting osmolar support to regulate water retention.

Collaterally, vasopressin, or antidiuretic hormone, produced in the hypothalamus and released by the pituitary gland in response to a decrease in blood volume exerts a pressor effect and acts as a diuretic by reducing the volume of urine, thereby conserving the volume of blood. That the caliber of blood vessels, for example, is adaptive locally as well as systemically demonstrates that control is effected by a hierarchy of control loops wherein those subordinate interact with those progressively more encompassing until the center just above the brainstem is reached. Central mechanisms initiate the release of circulating vasoconstrictors or vasodilators that cause the linings of blood vessels to contract or relax in response to the condition of the circulatory system, which includes cardiac output, partial pressures of oxygen and carbon dioxide, and the existing concentration of hormones and electrolytes in the blood.

The system is ambulatory and functions around the clock without control by the patient, who may be asleep. In an emergency not programmed for response distant from the clinic, a preplaced pump-pack can transmit the emergency signal to be activated by remote control. The jackets to be described can be placed in encircling relation about ductus along the digestive and/or urogenital tracts, the vascular tree, and/or the airway, to form a continuous passageway through the lumen of a synthetic line and into the native lumen without significant leakage or trauma and with no portion of the junction endoluminal, or projecting into the lumen. This capability has implications for the treatment of disease on a continuous, automatic, sustained, and when necessary, immediately adaptive basis.

This because the body consists of tissue pipelines and the tissues these supply. Except for absorption through the skin and oral mucosa, all intake into the body is through ductus. Any tissue can be accessed through ductus; when a side-entry jacket can be placed at a level that substantially excludes other tissue, the tissue that will be supplied is effectively isolated for targeted delivery of medication. Moreover, because the wall surrounding ductus support many biochemical interactions and discharge sensory feedback signals that modulate the control of numerous functions, the ability to circumscribe only a certain segment along a ductus for the delivery of drugs can have significant physiological implications, the more so when that segment is diseased.

In addition to communication affected by the autonomic nervous system, the luminal wall can release signaling proteins, such as chemokines and interleukins, and the luminal contents can include enzymes, hormones, cells containing cytokine signaling proteins, and so on, so that remote tissues are affected as well. As a result, there is no disease in which bodily conduits are uninvolved. No bodily conduit is analogous to inert plumbing; all are integrated into a hierarchy of negative feedback loops from the individual cells to the brain to actively and appropriately interact with the constitution, pressure, and velocity of passing contents.

The atrial walls, aortic, and carotid sinus bodies (glomus caroticum, carotid glomus) contain chemoreceptors that detect blood gas and acidity levels, which transmitted to the medulla, signal the autonomic nervous system to adjust the respiratory and heart rates and the stroke volume. Similarly positioned baroreceptors, or pressoreceptors, detect the blood pressure, likewise transmitted to the brainstem, which regulates subsidiary feedback control loops. Placed along an artery, the level at which a simple junction jacket such as that shown in FIG. 2 and described below is positioned sets the supply territory or region.

Advancing the jacket along the artery toward its end supply excludes more proximal branches to neighboring tissue, closing in upon and so narrowing the target zone or supply territory. By the same token, retreating along the artery admits side branches to neighboring tissue, thus expanding the zone. The junction bidirectional, antegrade delivery into the native lumen, whether vascular, digestive, urinogenital, respiratory, for example, is usually of a drug, whereas retrograde delivery from the lumen is usually of a diagnostic test sample.

Accordingly, automated ambulatory systems of pumps able to individually deliver any of a number of different drugs to jackets placed at different levels along a single ductus, different ductus belonging to the same bodily system, or ductus belonging to different bodily systems according to a programmed schedule and mediated by sensor implants have the potential to treat morbidities and comorbidities in a discretionary manner whereby each drug is delivered to the target tissue in a time coordinated sequence. Such treatment has the potential to outstrip any therapy dependent upon the systemic, hence, necessarily indiscriminate, administration of drugs. Susceptible to primary disease, and supplying and draining every part of the body, the treatment of bodily conduits has application to any localized condition.

Drug delivery through a side-entry jacket allows the upstream ductus and tissue it supplies to be avoided. When more effective, the drug can be increased in concentration for the target tissue while substantially reduced in dose compared to the systemic dose that would be needed to achieve the same dose at the target. Whether through the use of a reversal agent or an extraction-jacket, as will be described, if necessary any residue of the drug can be truncated from further circulation at a segment cutoff level. When a bodily conduit or ductus (singular) is itself diseased, effective and efficient treatment requires that medication be actively drawn into, not merely pass by it through the lumen with little uptake.

For disease within the wall of the ductus itself, the junction is extended to incorporate a magnetic collar of which the field strength is incrementally increased in the antegrade direction to achieve a more uniform penetration. Mechanically and magnetically based, the drug targeting spoken of here averts the contingency of discovering a substance that depends upon intrinsic properties and affinities for targeting therapy at the gross anatomical level. A drug must, for example, inhibit a destructive enzyme produced as the result of a genetic defect, such as the tyrosine hydroxylase inhibitor imatinib mesylate (STI-571; Novartis Gleevec®) to selectively target cancer cells.

Or it must take advantage of an inherent affinity of an organ or gland for a substance, such as the thyroid gland for iodine. Here instead, the drug is contained while conducted to the treatment site, where it is forcibly drawn into the surrounding tissue, regardless of its inherent proclivities. Allowing the medication to pass lesions within the wall surrounding the lumen, or ductus-intramural lesions, without uptake wastes medication that if targeted would have contributed to an effective dose, exposes healthy tissue downstream to the wasted dose, and results in complications.

Moreover, increasing the dose to achieve better absorption only increases the waste and the risk. Drug targeting substantially limits exposure to the drug to the tissue intended, isolating the drug from other tissue targeted elsewhere in the body by the same control system. This makes it possible to target a transplant organ without exposing the entire body to immunosuppressive or immunomodulatory medication, and can significantly reduce if not eliminate the damage to the immune system done by chemotherapy and radiation, for example.

The value of drug targeting with respect to the administration of immunosuppressive drugs, nonsteroidal anti-inflammatory drugs such as aspirin, which used to treat arthritis, for example, often produce gastritis and ulcers, statins that induce myositis in susceptible patients, steroids which can produce moon facies and induce diabetes, for example, and the avoidance of adverse side effects, drug-drug and drug-food interactions across the entire array of pharmaceuticals. All bode complications, making directly piped targeting significant in eliminating such adverse sequelae (see, for example, Polyak, B. and Friedman, G. 2009. “Magnetic Targeting for Site-specific Drug Delivery: Applications and Clinical Potential,” Expert Opinion on Drug Delivery 6(1):53-70).

Conventionally, magnet implants are limited to permanent magnets used to secure dental and maxillofacial prostheses and cochlear implants, and implanted rings used to ligate and atrophy tissue by compression ischemia. Other applications of magnetism require the use of an extracorporeal electromagnet to direct the magnetic field toward the treatment site, which limits such use to the clinic. The importance of drug targeting with respect to preventing rejection in transplantation, for example, will be addressed. Drug targeting can also be of value in averting side effects in drug tolerance and intolerance. Jacket placement assumes that the medication will be required on a long-term basis, would best not be taken orally, by injection, or injection that must be frequent as would promote patient noncompliance, and that accessibility to the site in order to implant the jacket and a port at the body surface to be described will not result in trauma more than negligible and transient.

When the dosage regimen frequent, and/or multiple drugs are needed making self-administration problematic, drug delivery is not dependent upon patient compliance but rather automatic as programmed, through a direct catheteric pipeline to the jacket or through plural lines respective of plural jackets from a port implanted at the body surface. At the same time, the port is available to administer another drug in the clinic from a syringe, for example. In order to realize the benefits of drug targeting, it is essential to possess means for establishing secure connections to ductus. The long-term indwelling of a catheter, needle, endoluminal implant, or prosthesis in a vessel often leads to adverse complications.

Subclavian, femoral, and internal jugular lines, and even peripherally inserted central catheters or PICCs, for example, are susceptible to infection, occlusion, breakage, and leaks (see, for example, Jumani, K., Advani, S., Reich, N. G., Gosey, L., and Milstone, A. M. 2013. “Risk Factors for Peripherally Inserted Central Venous Catheter Complications in Children,” JAMA Pediatrics 167(5):429-435; Barrier, A., Williams, D. J., Connelly, M., and Creech, C. B. 2012. “Frequency of Peripherally Inserted Central Catheter Complications in Children,” Pediatric Infectious Disease Journal 31(5):519-521; Shen, G., Gao, Y., Wang, Y., Mao, B., and Wang, X. 2009. “Survey of the Long-term Use of Peripherally Inserted Central Venous Catheters in Children with Cancer: Experience in a Developing Country,” Journal of Pediatric Hematology and Oncology 31(7):489-492).

Due to the risk of injury, air embolism, or the formation of a hematoma, maintaining multiple such diagnostic sampling and/or drug delivery points in different veins with indwelling catheters is not feasible, certainly not in an ambulatory patient, much less in one who is very young or very old. Moreover, even though direct access to the blood supply to an affected organ or region would afford considerable advantages both diagnostically and therapeutically, this cannot be done with respect to small much less major arteries, wherein the blood pressure is greater. However, the ability to form several secure junctions with arteries, even large ones, opens the way for targeting medication to, taking draws from, and inserting a diagnostic probe into the blood supply of the organs or tissues these supply.

Extended Capabilities

For less power demanding applications, power is obtained by carrying charged button cell batteries to replace the one or more in the surface port. Higher demand on a continuous basis calls for a larger implanted rechargeable battery, the surface port then used to take power from an electrical outlet. The need for more power on an intermittent basis can be satisfied by connection to an external power source with or without recharging a battery. Transdermal energy transfer allows direct tetherless delivery of power whether a battery is simultaneously recharged within a circumscribed area.

Most applications of ductus side-entry connection jackets simple and direct, a secure means for forming a junction with a ductus allows the application of a body area network with wireless transmission, or telemetry, even combined with transdermal energy transfer (see, for example, Mao, S., Wang, H., Zhu, C., Mao, Z. H., and Sun, M. 2017. “Simultaneous Wireless Power Transfer and Data Communication Using Synchronous Pulse-controlled Load Modulation,” Measurement (London, England) 109:316-325; RamRakhyani, A. K. and Lazzi, G. 2014. “Interference-free Wireless Power Transfer System for Biomedical Implants Using Multi-coil Approach,” Electronics Letters 50(12) 853-855; Yazicioglu, R. F., Torfs, T., Penders, J., Romero, I., Kim, H., and 4 others 2009. “Ultra-low-power Wearable Biopotential Sensor Nodes,” Conference Proceedings, Institute of Electrical and Electronics Engineers Engineering in Medicine and Biology Society 2009:3205-3208; Yoo, H. J., Cho, N., and Yoo, J. 2009. “Low Energy Wearable Body-sensor-Network,” Conference Proceedings, Institute of Electrical and Electronics Engineers Engineering in Medicine and Biology Society 2009:3209-3212; Young, D. J. 2009. “Wireless Powering and Data Telemetry for Biomedical Implants,” Conference Proceedings, Institute of Electrical and Electronics Engineers Engineering in Medicine and Biology Society 2009:3221-3224; Panescu, D. 2008. “Wireless Communication Systems for Implantable Medical Devices,” Institute of Electrical and Electronics Engineers Engineering in Medicine and Biology Magazine 27(2):96-101; further references provided below) to afford immediate diagnosis and targeted drug delivery at multiple locations under automatic control.

In such a hierarchical control system, the processors of a monolithic integrated circuit or microchip multicore microcontroller are partitioned to support one control node each, that programmed to function at the highest level of control as the master node sent the inputs from and having a time horizon comprehensive of the subordinate nodes, of which each contributes inputs to the pumps and jackets of the set based upon the sensors that feed it. Various arrangements possible, separate microcontrollers can be assigned to each node in the control tree.

The object is to optimize drug delivery while least interfering with freedom of movement. The sensors, the positioning of these, and the control desiderata among the nodes signaling a pump-pair depend upon the disease under treatment and vary widely. Once the implanted elements have been placed, only the need to replenish one or more drugs by injection through the body surface port and into a pectoral reservoir or replacement of the vial in the pump pack interrupt the patient in free movement.

Much as a vaccine confers artificially acquired immunity, such a system effectively serves as an adjunct or nonintrinsic suppressive or negative feedback response loop for adapting to an anomalous condition. By comparison, automatic ambulatory insulin pumps deliver insulin subcutaneously, intramuscularly, hence, systemically following a time delay, without targeting ability, and intravenous drug delivery is unsuited to an active life. Implant cardioverter defibrillators deliver electrical current, not fluid drugs, and ventricular assist devices provide mechanical action. Many genetic defects result in a failure to produce an essential enzyme or protein, or to produce the substance in the normal form and/or amount.

A disorder response system that supplements or substitutes for a defective intrinsic response constitutes what is in effect a physiological prosthesis, whereas a system placed to compensate for a genetic defect that evokes no innate adaptive mechanism is bionic. An automated prosthetic disorder drug delivery response system can function as a backup immune system to compensate for deficiencies in intrinsic adaptive responses, and where an intrinsic response is not just deficient but entirely lacking, such as where due to an inborn error in metabolism an essential enzyme is not produced, the automatic disorder response system can be characterized as bionic.

The distribution of control between the brain and subordinate circuits and ganglions a salient feature of the nervous system, such a hierarchical scheme may be seen as analogous to the relation between the microprocessor as master controller and the cortex and subsidiary or more localized control circuits in the spinal cord, for example. Here such a control tree receives feedback from the sensors associated with each jacket to feed data up the hierarchical levels continuously to adjust and coordinate the dosing of the drug delivery program both in detail, and overall.

While ground level subordinate or ‘intimal’ nodes associated with ground level sensors feed into a channel of control usually dedicated to one of plural morbidities, the same approach can be applied to organs, organ systems, lesions, or nidi. In any case, once processed by the subordinate nodes, or controllers, and the sensor readings have been passed up to the master node to apportion the release of drugs and/or other therapeutic measures among the targets, the consequence should best approximate normal homeostasis across the targets for the patient. Homeostatic optimization for a specific patient means that if present, deficiencies and defects of anatomy or physiology can prevent the realization of optimal homeostasis that would be possible were such obstacles absent.

Many genetic defects result in a failure to produce an essential enzyme or protein, or to produce the substance in the normal form and/or amount. A disorder response system that supplements or substitutes for a defective intrinsic response constitutes a physiological prosthesis, whereas a system placed to compensate for a genetic defect that evokes no innate adaptive mechanism may be thought of as ‘bionic.’ A disorder response system that supplements or substitutes for a defective intrinsic response might be referred to as a physiological prosthesis, whereas a system placed to compensate for a genetic defect so that no innate adaptive mechanism could be evoked is bionic.

That is, such an automated drug delivery system can function as a prosthetic disorder response system to compensate for deficiencies in intrinsic adaptive responses, and where due to an inborn error of metabolism the intrinsic response simply does not exist, as a bionic disorder response system. A prosthetic disorder response system best mimics or parallels that innate, and a bionic system best simulates an innate system.

In a tertiary medical center with the patient stationary, this scheme can be expanded so that diagnostic sensor feedback initiates and regulates not only ongoing dosing from among clinician prescribed drugs loaded, but can select as well as deliver drugs from among an unlimited number of drug supply reservoirs.

While the drugs delivered must be compatible, which is readily accomplished when delivery is targeted, such a system seeks to detect and return diagnostic information, such as at the level of metabolites, antibodies, antigens, and organic or inorganic substances, in relation to homeostatic balance without necessarily ascribing combinations of imbalances to a particular syndrome. Fully implanted and power and drug pack portable systems are loaded with a set of specific drugs to treat a diagnosed or predictable condition. To accommodate unpredictable as well as predictable eventualities, one factor deciding which drugs to store is broad spectrum, the applicability to treat an array of disease conditions.

By contrast, a stationary system need not be limited thus and does not require a preestablished diagnosis, so that correction expeditious, the risk of misdiagnosis is less. The direct delivery of drugs without relationship to a specific diagnosis allows immediate response to reasonably predictable intercurrent disease, especially valuable when comorbidities are likely. For example, with no change in behavior, metabolic syndrome, or the combination of abdominal obesity, hypertriglyceridemia, lowered high-density lipoprotein serum level, elevated plasma fasting glucose and low-density lipoprotein levels, and hypertension, progression to diabetes and cardiovascular disease is predictable, but not as to time of onset.

Such represents the internalization and rendering immediate of point of care detection (see, for example, Chikkaveeraiah, B. V., Bhirde, A. A., Morgan, N. Y., Eden, H. S., and Chen, X. 2012. “Electrochemical Immunosensors for Detection of Cancer Protein Biomarkers,” ACS [American Chemical Society] Nano 6(8):6546-6561; Rusling, J. F. 2012. “Nanomaterials-based Electrochemical Immunosensors for Proteins,” The Chemical Record 12(1):164-176; Rusling, J. F., Kumar, C. V., Gutkind, J. S., and Patel V. 2010. “Measurement of Biomarker Proteins for Point-of-care Early Detection and Monitoring of Cancer,” The Analyst 135(10):2496-2511; Choi, Y. E., Kwak, J. W., and Park, J. W. 2010. “Nanotechnology for Early Cancer Detection,” Sensors (Basel, Switzerland) 10(1):428-455. Liu, G. and Lin, Y. 2007. “Nanomaterial Labels in Electrochemical Immunosensors and Immunoassays,” Talanta 74(3):308-317).

For such patients with both portable and stationary systems, prepositioning sensor implants to detect and loading the stationary dispensing system with drugs to treat the additional symptoms associated with congestive heart failure, for example, allows the system to respond to these additional symptoms upon onset. Large in number, with additional drugs appearing often, the complement of drugs dispensed by such a stationary system is reduced to those for each purpose which clinical trials have shown to be safe and effective. The automatic drug selection and delivery control program or prescription data switches the drug reservoir catheters connected to each target ductus from among an unlimited number of drug supply reservoirs. In this, a body area network under ‘intelligent’ complex or hierarchical adaptive control can also be made to transmit data through a wireless network.

More than a single subordinate control level exceptional, a pump-pair and jacket set that includes three jackets, for example, requires a microcontroller with at least four cores (referred to by Parallax, Inc., whose multicore microtroller chips have the individual cores arranged in a circle or ‘hub’ for access to shared memory, ‘cogs’). Magnetic gradient-incorporating side-entry jackets, or piped impasse-jackets, already capable of drawing superparamagnetic carrier bound drugs radially outward through a ductus wall, patch-magnets are placed not to encircle ductus, but attached to the outer capsule of an organ supplied by the ductus and subject to the disease process under treatment. In most instances, the sensors are packaged in the form of stays configured for concentric insertion into the wall of the ductus or parenchyma before the jacket or patch-magnet is applied, so that these sit beneath or within the jacket or patch-magnet.

Minute diagnostic sensor implants respective of each jacket, patch-magnet, or other type implant provide feedback to the lower level nodes respective of each jacket, patch-magnet, or other type implant feedback site, thereby adjusting the dose of the drug respective of each within the prescribed drug delivery context, or the prescription as maintained by the master node. Highly stable conditions may require no more than one sensor closed feedback loop if any. Significant cost reduction may be achieved by limiting control software and hardware to the nonadaptive where more complex control and artificial intelligence are unnecessary. Whether control is nonadaptive or complex, the drivers remain standardized interchangeable pump-pair and jacket set open loop-driven stepper motors. The program automatically and immediately adjusts the delivery of medication for the present condition.

To cover different ranges of disease severity, the multicore microcontroller stores more than one program or prescription. Upon receiving appropriate sensor feedback through one or more subordinate nodes, the master node automatically transfers the program for the out of range node and jacket or the entire set. Should the feedback signals reflect a condition outside the drug delivery response range of the apparatus, a wireless body area network transmits an alarm to the clinic by emergency band or a conventional communication means, such as a text message. Depending upon the urgency, the input can be applied to dispatch an ambulance, alert the patient to return to the clinic, or instruct the patient to connect the pump-pair intake turret lines to higher capacity tabletop drug reservoirs containing the same or different drugs and switch to a different prestored control program.

The set produced as a unit, a single jacket set omits pump-pair outlet turrets, while multi jacket sets with a reasonable limit of four jackets provide pump outlet turrets to allow switching the pump outlets to any one jacket at any one time. More elaborate line switching as would permit simultaneous outlet switching to more than a single jacket at a time is possible but elusive of practical medical purpose, needlessly complex and costly, and inviting human error. In any such set, the lines connecting the pump-pair to each jacket is permanently fastened to the main and sidelines of each jacket, pump outlet switching among jacket inlets in the set accomplished at the pump outlet turret where any line to any jacket in the set can be rotated into alignment with the pump outlet.

A pump or pump-pair and jacket set thus constitutes a unit apparatus, of which portions proximal to the port implanted at the body surface remain outside the body, or extracorporeal, with those distal to the port implanted, hence, intracorporeal. Since individual jackets in a given standardized pump-pair and jacket set can be different sizes, can be placed along different type ductus in different parts of the body, and the one pump-pair supporting the jacket set allows the delivery of any drug to any jacket in the set in any sequence at any time, to further admit the inter-switching of lines among different pump and jacket sets only causes confusion. Jackets belonging to different pump-pair and jacket sets can be interposed with drug delivery times controlled by the multicore microcontroller in the multipump-pair power and control housing, or base into which the pump-pair plug-in modules insert. However, the need for more than one such set should prove rare and limited to cases of severe multiorgan disease or extensive injury.

The implanted system of catheters and ductus and tissue connectors to target drug delivery requires prosthesis-to-native tissue junctions which will remain secure and to the extent possible, conform to neighboring structures in a compliant manor without becoming disoriented. Other attributes essential for long term sufficient service include unsusceptibilty to the development of leaks, microbial intrusion, or injury to the substrate ductus or tissue to which the connector is mounted. These connectors must also be maintainable by means of inmate accessory channels that allow direct access without the need for reentry, even endoscopically.

Specifically, connection for securely and least disruptively merging catheteric drug and blood pipelines, or druglines and bloodlines, and native lumina is described in copending nonprovisional applications Ser. No, 14/121,365 entitled Ductus Side-entry Jackets and Prosthetic Disorder Response Systems, filed on 25 Aug. 2014 and its continuation-in-part Ser. No. 15/998,002. This application was directed to the creation of passages between synthetic and native ductus and the reverse by means of dependable connectors and durable connections able to remain in place indefinitely without damage to the substrate ductus, and if necessary, adapt to growth over a period of years.

Another application, Ser. No. 14/998,495, now U.S. Pat. No. 11,013,858, entitled Nonjacketing Side-entry Connectors and Prosthetic Disorder Response Systems, also addressed means for securely fastening the distal ends of catheteric pipelines, injection needles, electrodes, and miniature diagnostic and therapeutic probes, for example, to the surfaces of nonductal tissue such as that of the solid organs and glands for an indefinite if not lifelong treatment of chronic disease. As must ductus side-entry jackets, side-entry connectors must provide a synthetic-to-native junction which is durable, positionally stable, will accommodate growth, remain durable and leak-free, allow the direct delivery of drugs into the connector nor induce atheromatous degeneration in the substrate ducts, such a system could not remain implanted over more than a brief period in the clinic.

For a sustainable and durable ambulatory automatic response system, such connectors are a central prerequisite, indeed, an existential necessity. Both of the applications cited delineated the assignment of the axes of control in a fully implanted hierarchical control system to different morbidities, organs, or organ systems in the treatment of comorbid disease. The term ‘comorbid’ is intended to denote coexisting disease conditions whether or not these are directly related or cooriginal. Nonjacketing side-entry connectors extend this capability to structures such as the heart, stomach and colon, which abruptly motile and large in diameter, need not be jacketed or collared, as well as to nonductal tissue, prompting revision of the title from ‘nonductus’ to ‘nonjacketing.’

The junction created may be conventional and singular or support one in a number of disease process treatment control axes or channels of an automatic ambulatory prosthetic disorder response system placed to act as a backup ‘immune’ system.' Autonomic motor assist devices mentioned in passing are deferred for full description in an application to follow, that present concerned with electrical and pharamacological applications of nonjacketing side-entry connectors.

Such a lower to higher levelled control system is an adaptive ambulatory hierarchical prosthetic disorder response system, distinct from a conventional system such as a continuous glucose monitor which injects insulin as the need therefor is detected. With such a rudimentary device, the injection of insulin remains intramuscular and its dispersal systemic. In contrast, a system of the type meant is fully implanted, and the insulin is not dispersed but released through a side-entry jacket directly into the portal vein as would a normal pancreas.

This eliminates the need for the intramuscular injection of an insulin, with a time lag that compared to release targeted thus is considerable. No subcutaneous injection, oral antihyperglycemic drug, or inhaled formulation of insulin, metformin, or any other drug can approximate the ability of an instant response system with multisensor input under hierarchical control to modulate blood glucose to within the normal range. Insulin overdose or overproduction should it arise is remediable by releasing metalloprotease insulin-degrading enzyme (insulysin, insulinase) or glucose directly into the hepatic portal vein or glucose into the bloodstream.

Because the automatic disorder response system is equipped and programmed to maintain its own components as well as to monitor and treat the disease, provided the drugs required if any are replenished as necessary, and except for periodic charging, usually by means of transdermal energy transfer, it is meant to function autonomously for years. To treat symptomatically complex comorbid disease, which may elude diagnosis, such a negative feedback system assigns lower level closed loops to the control of individual symptom values, such as characterize a key metabolic pathway or process. The conventional treatment regimen established, inputs from symptom or variable sensor implants provide feedback, to which the controller responds by adjusting the delivery in dose level and interval of pharmaceutical and/or electrical therapy to recover to the programmed target set point as the normal value.

Where the regimen is unestablished, different drugs and electrical discharge patterns are first established in the clinic. In the treatment of comorbid disease, higher level control is applied to monitor the summary or overall homeostatic condition and if necessary, apply adjustments among the control axes, such as to shift subordinate set points when necessary. Such a system is fully, or closed-skin, implanted, a belt-worn battery pack such as those used with conventional implanted assist devices required only when the simultaneous treatmeny of multiple comorbidities creates a demand for power too large to be satisfied by transcutaneously, or transdermally, recharged implanted or body surface port-held button cells.

If and only if necessary, an extracorporeal battery pack is connected to the intracorporeal components through a body surface, or on-the-skin jack, socket, or port, designed to be easily kept sterile, different types described and illustrated in copending applications Ser. Nos. 14/121,365 and 15/998,002, entitled Ductus Side-entry Jackets and Prosthetic Disorder Response Systems, FIGS. 27 and 28 and Ser. No. 16/873,914. entitled Vascular Valves and Servovalves—and Prosthetic Disorder Response Systems, FIGS. 26A thru 26C.

Where the means for achieving the overall homeostatic condition closest to that optimal, meaning closest to that normal, among an unfamiliar combination of comorbidities is unclear, the level-by-level ‘bottom up’ data buildup of the self-optimizing and diagnostic empirical approach applied by the control system allows it not only to treat and diagnose a complex condition but to actively and methodically seek out and depending upon the combination of drug onset times, eventually define this condition. With such a system, this process of self-optimization proceeds without interruption until resolved, undegraded by lapses between blood draws or other intermittent tests and unconstrained by limitations of time and staffing in a clinic.

The system functions continuously through stress testing, to include iatrogenic to ascertain the relative value of different drugs under such conditions. More broadly, the system can be programmed to determine and report the optimal dose of any drug previously not deliverable as isolated by direct pipeline targeting. A lucid wearer who documents events such as periods of exercise and sleep can further aid in clarifying the relative performance of different drugs during such periods. In that the detection of unsensed involuntary dysfunction whether of autonomic motor or metabolic function cannot depend upon patient awareness, the dependency upon patient perception to elucidate the nature of the complaint is eliminated.

For this reason, a system that automatically responds to unsensed malfunction, an implanted backup ‘immune system,’ must initiate remedial action immediately on the basis of sensor inputs without the participation of the patient. Unsensed aberrations of plysiology must be entrusted to sensor inputs chosen and positioned to detect indicia associated with the disorder or disorders and to implanted electrical, mechanical, and chemical effectors. Side-entry jackets and connectors can fix the position of sensors that would otherwise lack positional stability, at the same time delivering drugs and/or electrical current to the site of implantation.

When the patient is likely to harbor secondary or additional disorders at a later date, an initial procedure responsive to incontinence, just as any to treat a singular disorder, is responded to by initial treatment using componentry that except for placing a body surface rather than subdermally positioned injection port, or portacath, allows the introduction of additional control channels as may later become necessary. If secondary or sequelary morbidity is likely to affect the same region or organ and the connection—unlike one that conveys blood or a drug to be delivered continuously—the different viewing, diagnostic, and therapeutic devices to be used are of like diameter, allowing these to be inserted interchangeably through the aperture at the center of the nonjacketing side-entry connector.

Depending upon the individual patient and the number of drug delivery components likely to become necessary at a later date, a tissue expander to create more space for the small flat drug reservoirs can be placed in the pectoral or pelvic regions, for example. The drug delivery components, include a subdermal surface port, catheteric pipeline from the port to a drug reservoir with outlet pump controlled by the microcontroller, or in comorbid disease, the microprocessor, which to allow the retrieval of biopsy test samples, can be reversible, and the terminal connector—ductus side-entry jacket or nonjacketing side-entry connector to the tissue to be targeted.

Of these, when larger in number, the drug reservoirs are preferably retained within one or more surgically constructed pockets positioned subdermally in the pectoral or pelvic regions. An object of good design being to spare the patient the impediment of a belt-worn pack, intracorporeal positioning thus, provided it does not impose undue discomfort for the patient, is preferable. In a more elaborate system to treat several comorbidities, the need for an extracorporeal belt-worn pack may be unavoidable, so that in this situation, the drug reservoirs can be housed within the same enclosure as the batteries that serve as power source. To assure its immediate sighting, the free proximal end of the catheter (line, feedline) is crimped with a magnetically susceptible ferrule marked with contrast, such as tantalum-based.

If the number of drugs to be provided for the same or different disease necessitates, an external port with multiple openings, each clearly marked is used. Prepositioning a connector with piping and electrical conductor or conductors and control electronics—but not a portacath, reservoir, or pump, which can be placed later—allows testing electrostimulation as the first and best option. Not requiring a portacath, reservoir, or pump, for example, electrical means involve the fewest components, take up the least space, and generally allow placement with the least dissection. Provided unintended function of like innervation is unaffected, the lead or leads can be positioned at a functionally and anatomically higher level. A sacral neuromodulator, for example, may exert an effect on rectal as well as bladder function where only one or the other called for treatment.

In such a circumstance, more highly resolved stimulation farther along the neural circuit once the nerve divides to send the target organ its respective branch or ramus is therapeutically selective in eliminating unwanted concurrent stimulation of another organ, such as the rectum where the bladder had been intended. Then, however meticulous was the testing before implantation, even tiny movement of the lead, whether tined or barbed, cannot shift the distribution of stimulation. Using an electrode, lead, or leads to stimulate the innervation, electrostimulatory neuromodulation is least invasive of a native sphincter, and least susceptible to the complications associated with pharmacological treatment, to include adverse side effects, drug-nutrient, and drug-drug interactions. When the likelihood of secondary disease is high, the need to reenter is best avoided by prepositioning the additional components that would become necessary to treat these.

At the same time, including at the outset fluid, meaning drug, blood, or urine catheteric pipelines that may later become necessary allows for the addition of other components as necessary without the need to reenter the patient in order to add or replace the connector or to place additional lines at a later date. Initial placement best enables the delivery of treatment beyond that contemplated at the outset, not just to allow adjustment in a single therapeutic modality but in the modality or combination of modalities. The concept of making it possible for the therapy to be adjusted without the need to revise the initial procedure or replace the original implants at a later date applies not just to disease able to induce secquelary pathology but to specific disorders for which the best therapeutic regimen will need to be adjusted, as well as when the optimal result can be found only through empirical testing.

To cite one instance, with a refractory gastric reflux that resists treatment with a proton pump inhibitor and/or induces unwanted side effects at the oral (systemic) dose necessary, delivery through a side-entry jacket or nonjacketing side-entry connector at the lower esophageal (cardiac, gastroesophageal sphincter, at the gastroesophageal junction allows the dose to be increased to a level that if circulated could result in anchlorhydria, or an insufficiency of hydrochloric acid in the gastric juice as is necessary for the normal breakdown of food and digestion. Copending application Ser. No. 14/998,495, entitled Nonjacketing Side-entry Connectors and Prosthetic Disorder Response Systems, showed that the semicircular needles used to securely anchor the connector into the substrate tissue could be electrified in a number of different electrical discharge patterns.

This feature afforded the ability to vary the delivery of current concomitant with the piped delivery of different drugs. If the decision is made to resort to electrostimulation of the sphincter, in lieu of or in combination with medication, the connector, already in place, can be used to test numerous modes of electrical pulsation or drug based treatments with or without concurrent or intermittent electrostimulation. The ability to directly target familiar drugs, hormones, and enzymes allows the use of these in novel ways that can advance pharmaceutical science equivalent to the development of new drugs.

Where a different etiology would reasonably effect these different component modes of treatment in a distinctively different way, existing electrostimulators, or neuromodulators, such as sacral and gastric are limited to electrostimulation of the innervation; usually at a high enough level as to involve unintended tissue.

Conventionally, diabetes, which affects the entire body, is treated separately, whereas here, the systemic therapy is certainly provided but also locally coordinated with means to remediate the local consequences of the systemic disorder. Damage to the vagus nerve may be uninvolved in some gastroparesis, or may have resulted in other damage to the stomach, so that only to electrostimulate the nerve would never afford a cure. In fact, the condition is usually treated pharmacologically as well, but without the benefit of direct pipeline targeting which eliminates constraints of dose.

In most cases, the more detailed components of the condition will not be known; however, empirical adjustment to determine the optimal combination of electrostimulatory and pharmaceutical curative factors will not only serve to more effectively ameliorate the medical problem but help to explain its basis. In this process, the fact that the electrostimulation and drugs are precisely targeted eliminates the host of detractive factors contributed by exposure to the drugs and electrostimulation of extraneous tissues and organs. The pathophysiological analysis as to etiology and optimal treatment regimen for a given condition in a given patient are hindered by the number of variables, which is only further complicated when extraneous tissue is involved.

Analogous application to dysmotility along the gut or urinary tract is intentional. Complicated conditions may necessitate a coordinated response that addresses collateral conditions elsewhere within the same or in other organ systems.

The satisfactory application of a therapeutic regimen which senses the need for and automatically actuates a coordinated response that includes directly targeted electrical discharges and/or drug delivery, as well as autonomic motor assist devices, requires and justifies the placement of a microcontroller, sensors, and other components necessary to provide such a coordinated response. Administered conventionally, proton pump inhibitors taken orally often fail to afford sufficient relief of acid reflux or of gastroparesis, and prokinetic, or promotility, drugs, such as erythromycin, domperidone, metoclopramide, (Camilleri, M., Parkman, H. P., Shafi, M. A., Abell, T. L., Gerson, L. and the American College of Gastroenterology 2013, Op cit.) which may be injected with an endoscope, have yielded unsatisfactory results for the long term relief of gastroesophageal reflux, as have hormonal and antinausea therapy.

In addition to the increased utility of drugs that must not be administered systemically at a dose limited by the need to avoid adversely affecting other tissues, automatically targeted delivery at intervals of a short duration drug such as botulinum toxin type A to a sphincter, for example, elevates it in utility from a temporary palliative, means of confirming a diagnosis, and possibly averting a surgical procedure to a sustainable source of relief.

It should be assumed that the need for additional druglines will develop overtime; especially if the insertion site is deep as will detain revision, fluid lines (catheteric drug pipelines) connected to the side-entry connector are routed to minimize the risk of organ strangulation. At least until the need therefor arises, these should be tunneled subdermally, so that the free proximal ends are prepositioned for connection to drug delivery components once these become necessary. If the number of drug target sites exceeds the number of subdermal ports acceptable, then a body surface type nonjacketing side-entry connector as described in copending application Ser. No. 14/121,365, entitled Ductus Side-entry Jackets and Prosthetic Disorder Response Systems is placed.

Such a surface port can be placed temporarily during the initial drug testing period. If the number of drug target sites is reduced, the external port connecter is removed and replaced by portacaths. The need to continue with an external port connecter is limited to comorbidity that poses numerous electrostimulation and/or drug target sites. Provided the systemic medication previously used provided some relief, the treatment commences with the same drugs, with, however, the dose adjusted for direct targeting, wherewith the exposure of unintended tissue ceases as a consideration.

To be driven forward from a body surface port implanted subcutaneously in the pectoral region, for example, to the target, small amounts of drugs can be placed at the head of a column of water, and can be arranged for intermittent automatic dispensing by varying the length of the water segments between successive doses. The reversible pumps used allow drugs to be withdrawn, the line and reservoir if present flushed clean, and another drug introduced.

The reason that such an assist device with electrical and fluid delivery capability is not placed at the outset is that to encircle the native sphincter requires an extent of circumesophageal dissection and the use of suture to stabilize the surrounding tissue and thus avoid producing the effect of sliding hiatus hernia. Primarily to prevent migration, autonomic motor assist devices such as an electromagnetic sphincter have suture pass-through loops such as those shown as part number 32 in the accompanying drawing figures.

These loops, at several points toward the proximal and distal margins, allow connection of the implant to the surrounding tissue, here to the diaphragm, the sphincter and diaphragm therefore moving together. The use of an electromagnetic sphincteric assist device should be viewed as a last resort; in all but a small proportion of cases, the patient would never require mechanical assistance, so that the need for revision would almost always have been avoided. Native sphincters open by shortening upon contraction.

This action is best stimulated electrically, and next best through the direct application of inotropic drugs through a nonjacketing side-entry connector. An electromagnetic sphincteric assist device does not function thus but applies constrictive force entirely about the native sphincter. Because this mode of constriction is different than that to which the native sphincter is adapted (see, for example, Theodosiou, N. A. and Tabin, C. J. 2005. “Sox9 and Nkx2.5 Determine the Pyloric Sphincter Epithelium under the Control of BMP Signaling,” Developmental Biology 279(2):481-490; Moniot, B., Biau, S., Faure, S., Nielsen, C. M., Berta, P., Roberts, D. J., and de Santa Barbara, P. 2004. “SOX9 Specifies the Pyloric Sphincter Epithelium through Mesenchymal-epithelial Signals,” Development (Cambridge, England) 131(15):3795-3804), an electromagnetic sphincteric assist device should always incorporate a fluid line for drug delivery to ameliorate any adverse sequelae of forcible constiriction.

A potential disadvantage of conventional electrtrostimulation is that the stimulation is applied to a larger nerve which intercepted at too high a level is likely to include fibers that will eventually ramify to tissue other than that to be treated. In most instances, a side-entry connector is local to the target tissue, so that affecting unintended tissue is out of the question. Whereas electrostimulators have limited prescribed points of insertion, ductus side-entry jackets and nonjacketing side-entry connectors can be placed at any nervous or vascular level to deliver any combination of electrical discharge and/or medication.

In the treatment of a sphincteric motor dysfunction, the resolution to be preferred is that simplest and most compact, beginning with electrostimulation through a nonjacketing side-entry connector with only an electrical wire, not a fluid drug delivery line or catheter. If inadequate, the addition of a fluid drug delivery line follows. If electrostimulation and direct drug targeting fail, then an electromechanical assist device is employed.

The longitudinal extent of a sphincter usually not affording sufficient space to position both a combination-form electromechanical sphincteric assist device with built in fluid and electrical capability and a nonjacketing side-entry connector, unless confidence in the side-entry connector is high, the nonjacketing side-entry connector should be placed first, just proximal to the sphincter, with the distal end of its catheter and/or electrode set to penetrate the sphincter proper.

Then, if placed, the combination-form electromechanical sphincteric assist device will be drug and electrical discharge capable, allowing the side-entry connector to be removed. If the patient history indicates little probability that the side-entry connector will work to satisfaction, the combination-form electromechanical sphincteric assist device is placed ab initio. The larger sphincters of the digestive and urinary tracts consist of specialized adluminal muscle fibers continuous with the surrounding tissue.

The electromechanical sphincteric assist device is placed to encircle the sphincter, the suture loops 32 such as those shown in FIG. 1 used to prevent unwanted mobility, in this case, equivalent to a sliding hiatal hernia. Provided to do so is not likely to result in erosions, ulceration, or fistulization of a sphincter lining such as that of the internal urinary sphincter which is unadapted to and intolerant of constant constriction, the electromagnetic sphincteric assist device type ductus jacket is placed just proximal to the native sphincter. The lining of the digestive tract much tougher and if not so intensely as a sphincter, routinely constrictive, when surrounding tissue or some peculiarity of the anatomy recommend, placement of cardiac, pyloric, and ileocecal electromagnetic sphincters are positioned just proximal or short of the native structure.

The surrounding tissue is dissected away if and only if the placement of an electromagnetic sphincteric assist device has been confirmed as necessary and not likely to cause injury that cannot be controlled through the delivery of medication through an accessory channel. Where separation from the surrounding tissue is disruptive, suture loops 32 in the accompanying drawing figures situated about the outer surface of the assist device are used to reattach the surrounding tissue.

The ability to apply any drug, drugs, and/or electrostimulation in any pattern of pulsation with a nonjacketing side-entry connector such as that shown in FIG. 9 and the further ability to mechanically force the motility required with the aid of a combination-form sphincteric ductus side-entry jacket, by its spectrum of treatment modalities and results found empirically through adjustment outside the body, allows dispensing with much prediction and testing to offset the cost of treatment.

While it may be presumed that once forcible closure is instituted, electrical and chemical modulation might just as well be disposed of as superfluous, because forcible closure, especially where the tissue is not adapted for it, often injures the conduit lining. In this circumstance, the sphincteric assist device best includes the capability to forcibly contract the sphincter only once electrical and chemical neuromodulation have been unsuccessful.

For this reason, a sphincteric assist device usually includes electrical and drug delivery means ab initio, allowing the use of force to be minimized through extracorporeal adjustment following closure, without the need for reentry or revision. Then, if neuromodulatiory means substantially close the sphincter so that only a final application of constrictive force is necessary to finally squelch acid reflux, the additional force is applied over the shortest interval following neuromodulation.

Thus, if the severity of the condition is recognized early, the placement of a sphincteric assist device with fluid and electrical delivery lines allows one time placement and the ability to adjust the therapy until that regimen most effective with the least treatment is determined. Then if medication and electrostimulation fail, the sphincter is forced shut. A comparable approach applies to the targeted delivery of digestive hormones, enzymes, and electrical neuromodulation to reverse gastric and/or intestinal hypo or hypermotility. The concurrent placement of sensors and control microcontroller allow the process of optimization and future adjustment as necessary to proceed automatically. When placed in conjunction with a robotically assisted procedure, use of a robotic or camera access port already present should be considered.

As to a LeVeen shunt, the ability to access the junction with the vein for delivery of drugs should substantially eliminate the complications of superior vena cava thrombosis, infection, variceal bleeding, and disseminated intravascular coagulopathy encountered with these devices. Ductus and nonjacketing side-entry connectiors are intended to remain in place over a long period if not permanently, thus supporting the long term functionality of a fully implanted prosthetic disorder response system that uses inputs from implanted sensors to govern the targeted delivery of drugs to different treatment sites under automatic control. The elimination from the vena cava, internal jugular, or any other vein of an indwelling catheter provides a safety advantage.

Equally important as these conventional applications, the stable connections, long life, and direct to junction delivery of drugs that can treat the disease and maintain the catheteric line means that ductus and nonjacketing side-entry connectors are able to support, and in so doing, make possible, an automatic control system as addressed in copending application Ser. No. 14/121,365, entitled Ductus Side-entry Jackets and Prosthetic Disorder Response Systems, filed on 25 Aug. 2014. Such a system, conceived of as a prosthetic backup immune system able to treat comorbid disease, uses implanted sensors to signal the need to target medication in the appropriate doses to an organ, vessel, or a combination of these.

Full implantation, automatic ambulatory operation, and system self maintenance have the potential to critically improve patient quality of life. Where indwelling catheters limit free movement, require frequent examination, and cause progressive irritation and injury that limit duration, ductus side-entry jackets form secure catheteric junctions with ductus, and nonjacketing side-entry connectors the same with organs and tissues, to minimize if not eliminate growing trauma at the entry wound.

An accessory channel to introduce catheter and target maintenance measures as needed is not accessed through a ‘piggyback’ port dangling out through a hole in the body wall but rather through a separate fully implanted or closed-skin portacath or secure port at the body surface, described in copending application Ser. No. 14/121,365. This allows apparatus such as nephrostomy tubes and central venous catheters previously limited to temporary use and the need for replacement if necessary to reman in place over a long term if not permanently.

Side-entry connectors are intended for long-term or permanent fastening of synthetic or tissue engineered ductus to or from native or transplanted organ or tissue, thus into the parenchyma. Ductus side-entry jackets allow secure connection to ductus, and so release, for example, drugs directly into the circulation rather than into the parenchyma. As such, both include secure fastening means, means for passing fluids and/or electrical currents through the junction, and an accessory channel to allow release into the line of medication to prevent the buildup of the clot, crystal accretion and biofilm as appropriate which have thwarted the long-term use of narrow catheter implants. Ordinarily, the line proximal to the outlet of the accessory channel does not convey biological but rather pharmaceutical materials, so that it is not susceptible to fouling or occlusive buildup.

Ductus and nonjacketing catheter-to-tissue and tissue-to-catheter fasteners that provide secure and leak-free attachment and can be accessed without invasive entry to deliver maintenance substances are indispensible for the implementation of ambulatory prosthetic disorder response systems. If necessary, counteracting agents, such as a solvent or antimicrobial can be included with the pharmaceutical at the inlet into the line. In addition to the growing irritation caused by movement at the tubing-tissue interface, synthetic tubing that is smaller in gauge placed in the vascular tree tends to be thrombogenic and subject to the formation of biofilm, and placed in the urinary tract, susceptible to crystal accretion, as seen in the need to periodically replace current ureteric stents.

However, clot, biofilm, and crystallization eliminated, a synthetic tube can remain in place indefinitely, is not subject to stenosis, degradation, or infection, has no need for a blood supply, has no intrinsic physiology that is mismatched when resituated to a different location, and is not obtained at the cost of a preliminary procedure that harvests and renders normal tissue abnormal. These factors have limited the time that catheters can be allowed to remain in place. Accordingly, side-entry connectors not only pass fluids as a primary object of placement, but incorporate an accessory line for self and catheter support.

Such junctions can be used to extend the indwelling time of catheters in otherwise conventional practice, but are essential for the implementation of automatic ambulatory prosthetic disorder response control systems as described in copending nonprovisional application Ser. No. 14/121,365. Long term stability and ease of maintenance allow, for example, the placement of drug targeting means in a primigravida requiring a drug that would harm the fetus, where the unobtrusive apparatus placed early in pregnancy can remain in place over the balance of her reproductive if not entire life.

Provided a reversal agent is available, incomplete takeup within the target organ or tissue can be accomplished through a second ductus side-entry jacket on the outflow vein or veins to deliver that agent, thus preventing continued transport through the circulation, access to this second jacket as specified above for an accessory channel Essential substances for which there is no reversal agent are prevented from further transport by introducing the medication in the form of a ferrofluid wherein the drug is bound to superparamagnetic nanoparticles drawn by magnets situated about the organ periphery from the point of entry into the surrounding tissue to draw the drug into the parenchyma or surrounding tissue.

Nonjacketing side-entry connectors are of two types, those for internal use as described herein and those for placement at the surface of the body, described in copending application Ser. No. 14/121,365. Reduction in the need for maintenance is advantageous for a patient of any age, but especially for those at the extremes of age and their caregivers. Whereas the object in forming a junction between a synthetic or tissue engineered tube and a native ductus, such as a vessel or a ureter, is to accomplish merging confluence with minimal shear stress, connection to solid or hollow organs and to fascia-invested muscle, for example, is usually to fixedly implant and if necessary, advance and retract a styloid or styliform, that is, a rod or needle-shaped device.

Such include electrodes; ultrasonic, electrohydraulic, and laser probes; scopes; and/or hollow (injection/aspiration) needles, hypotubes, lasers; and/or heating elements. Those implanted for therapeutic neuromodulation can be chemical, electrical, such as leads placed for transcutaneous electrical nerve stimulation, or these inserted side by side. Electrodes, for example, can be electroanalytic and/or electrotherapeutic, such as electroanalgesic, and different syloid or cabled devices can be positioned side by side.

In an automated system, the energization of these, individually or in coaxial or disparate combinations to treat singular or comorbid conditions, can be a part of or coordinated with chemotherapy, radiotherapy, or chemoradiotherapy in adjuvant and/or neoadvjuvant relation. That is, a connector for the immobile infixion to or within nontubular or nonductal anatomical structures must allow the connection as necessary of electrical lines and small caliber cabled devices or styliform components such as therapeutic and diagnostic electrodes or microelectrodes, lasers, or probes or microprobes in addition to fluid lines.

The ability to isolate or circumscribe an organ or region for treatment by pharmacotherapy, chemotherapy, radiotherapy (radiation therapy, radiation oncology), or chemoradiotherapy has the potential to eliminate much, perhaps all, of the adverse side effects, drug-drug, and drug-nutrient interactions associated with these treatment modalities. Photon radiation as in brachytherapy involves the infixion of seeds, wires, or pellets that move with the substrate organ or tissue and are therefore positionally stable without the need for a means of positional fixation. Use of a remote afterloader, which has limited applicability, and must be withdrawn leaving no radioactive substance in the patient, denies the ability to terminate the treatment based upon reexamination at intervals without the need to repeat the procedure.

In general, the ability to circumscribe, or isolate, a native organ, blood supply territory, or an organ transplant by means of placing side-entry jackets on the arterial inflow, and if necessary, the venous outflow, allows the restriction of side effects, if any, to the organ or tissue circumscribed. The targeting of a lesion within an organ or tissue is by placing a nonjacketing side-entry connector mounting a styloid device such as a catheter or hollow needle at a fixed angle and depth within the organ or tissue. The use of both jackets and a side-entry connector to treat the same organ or tissue then serves to directly target the lesion while furnishing a background dose to the surrounding tissue as ‘extension for prevention,’ while containing exposure to the tissue intended.

The same application describes radiation shielding with both short and longer half life radionuclides and other radioisotopes (see, for example, Murata, T., Miwa, K., Matsubayashi, F., Wagatsuma, K., Akimoto, K., and 5 others 2014. “Optimal Radiation Shielding for Beta and Bremsstrahlung Radiation Emitted by (89)Sr and (90)Y: Validation by Empirical Approach and Monte Carlo Simulations,” Annals of Nuclear Medicine 28(7):617-622; Bhattacharyya, S. and Dixit, M. 2011. “Metallic Radionuclides in the Development of Diagnostic and Therapeutic Radiopharmaceuticals,” Dalton Transactions 40(23):6112-6128; Yue, K., Luo, W., Dong, X., Wang, C., Wu, G., Jiang, M., and Zha, Y. 2009. “A New Lead-free Radiation Shielding Material for Radiotherapy,” Radiatiation Protection Dosimetry 133(4):256-260; Amato, E. and Lizio, D. 2009. “Plastic Materials as a Radiation Shield for Beta-Sources: A Comparative Study through Monte Carlo Calculation,” Journal of Radiological Protection 29(2):239-250; Jødal, L.2009. “Beta Emitters and Radiation Protection,” Acta Oncologica (Stockholm) 48(2):308-313; Papagiannis, P., Baltas, D., Granero, D., Pérez-Calatayud, J., Gimeno, J., Ballester, F., and Venselaar, J. L. 2008. “Radiation Transmission Data for Radionuclides and Materials Relevant to Brachytherapy Facility Shielding,”Medical Physics 35(11):4898-4906; Van Pelt, W. R. and Drzyzga, M. 2007. “Beta Radiation Shielding with Lead and Plastic: Effect on Bremsstrahlung Radiation when Switching the Shielding Order,” Health Physics 92(2 Supplement): S13-S17).

When flushing through the line with water would not preclude the risk of injury, tungsten shielding offers the best combination of light weight and expense. Tungsten is toxic and must be encapsulated for chemical isolation, polyethylene terephthalate and related polyesters suitable materials therefore. Implants accurately prepositioned to work in conjunction with external pencil beam radiation or other means of excitation from outside the body at intervals, such as radiofrequency magnetic field alternators to warm the implants, can represent strike-target reactive or relay emitter devices, receiving antennas, or discharge tubes for substances used in radiopharmaceutical practice such as nuclides, any of which can be fixedly prepositioned in relation to the target for energization by the external source with the aid of a nonjacketing side-entry connector.

The nonjacketing connectors described herein are intended to achieve positioning as stable and durable as reversibility with relatively little trauma will allow. When placement is temporary, the needles are smooth surfaced and provided with a snare-grab to facilitate extraction. The fine needles must be of extreme strength, hence, made of graphene, titanium, or heat treated 17-4PH and 15-5PH stainless steel, which martensitic however, are magnetic. If this will pose a problem, the needles are made of a cold worked austenitic stainless steel. The use of a nonjacketing side-entry connector assumes that positional stability is essential for a treatment to continue over a period long enough to work at all or to work to better effect.

Scheduled dosing with passive drug delivery necessitates patient or assistant compliance, whereas automated delivery does not. This factor becomes the more important as the number of drugs to be administered increases, especially if the administration thereof must be coordinated. A port with multiple openings fastened to the body surface is not considered an implant. Such a port, described in copending application Ser. No. 14/121,365, entitled Ductus Side-entry Jackets and Prosthetic Disorder Response Systems, can provide openings which are closed off to the exterior by injection bottle cap type elastomeric plugs or a lid that allows insertion of a line from an external pump. To admit a fiberscope or cabled device such as a fine excimer laser, for example, the plug is withdrawn from the port at the body surface.

The direct delivery of drugs to nidi that would ordinarily require passage through the liver is overcome by delivering the drug in its post-liver metabolized form. That is, the liver and/or kidneys bypassed, drugs ordinarily administered as prodrugs must be converted into the biotransformed (post-metabolized, post-hepatic, post-renal—and hypothetically, application to a fetus not yet practicable—post-placental) form exogenously before direct application at the treatment site. Similarly, for direct application, conventional drugs must be adjusted in dose. Some drugs thought to have no topical or direct effect upon tissue do in fact have such properties, a notable example being statins of which the direct healing effect is referred to as pleiotropic. Using the means described herein, drugs such as lithium, which is neuroprotective can be directly targeted to the treatment site, pre- or post-hepatically and/or pre- or post-renally.

That the immunosuppressant cyclosporine, also nephrotoxic (see , for example, Yu and Brenner, Op cit., pages 1703-1704), can be targeted to a non-kidney transplant, avoiding the kidney, is further addressed below in this section. With superparamagnetic iron oxide nanoparticles as drug carriers, one can selectively target diseased tissue within an organ or tissue while keeping the agent away from the healthy tissue surrounding the lesion. Such an application was addressed in connection with FIGS. 6, 13A, and 13B in U.S. Pat. No. 11,013,858.

Ordinarily, the use of a reveral agent or counteractant is complicated by the possibility of unwanted reversal; however, the segregation implicit in targeting allows conjugation or chemical reaction between the therapeutic agent and reversal agent to be controlled. Delivery of the therapeutic agent, and if needed, the reversal agent, can be pulsed or continuous. Lithium, for example, to treat bipolar (manic depressive, mood) disorder, can be routed directly to the brain and substantially kept away from the kidneys, intestinal tract, and thyroid, for example.

Targeting to the brain of lithium or another drug, or to the eyes of an ophthalmic drug is by direct delivery into the internal carotids through ductus side-entry jackets. When directly delivered to the brain, the risk of drug-induced renal complications, especially if counteracted when continuing through the circulation, is substantially eradicated. The smaller dose needed when not dispersed throughout the pre- or post-systemic circulation should prove harmless, but if needed, a counteractant, neutralizing, or reversal agent is delivered directly into the jugulars or internal jugulars. In this way, acetaminophen can be kept away from the kidneys and nonsteroidal anti-inflammatory drugs from the gastrointestinal tract of a patient with chronic or migraine or cluster headache (see, for example, Raskin, N. M. 2005. “Headache,” in Harrison's Principles of Internal Medicine, Op cit., pages 85-94).

The direct delivery to the brain of drugs averts metabolism by, and is limited to drugs that do not depend upon, conversion by the liver and kidneys, for example. Such drugs exercise the therapeutic effect locally at the site to which delivered, the brain exemplary in this regard. Where antecedent conversion of the drug is essential, administration of the drug through direct targeting must deliver the drug in its activated or effective post-metabolized form. For drugs with direct local action, dispersion in a relatively small and substantially isolated volume of blood conserves plasma concentration, minimizes the time to peak plasma concentration as a primary factor in clinical efficacy (Raskin, N. M. 2005, Op cit., page 91), avoids breakdown by nontargeted tissue, and minimizes loss through absorption which could induce adverse side effects.

This consideration, fundamentally important in the administration of chemotherapy, radiotherapy, chemoradiotherapy, and immunotherapy, all inducing severe side effects, is no less important in the administration of migraine medication, where the efficacy of the drug tends to vary in proportion to its toxicity. For example, when dispersed throughout the systemic circulation through injection or oral administration in systemic doses, sumatriptan, usually formulated to include naproxen, one of the most effective drugs for reducing the pain of migraine and one unlike a statin not in question as to its direct tissue contact efficacy, can induce serious side effects, to include venricular dysrhythmias, coronary vasospasm, myocardial ischemia, and infarction. Less serious neurological side effects include altered sensation of temperature, pressure, pain, paresthesias, and sleep disturbances.

The release of serotonin 1B, 1D receptor agonists, antiemetics, analgesics, for example, to suppress a migraine headache on inception depends upon the experience of an aura or prodrome by a competent patient able to control the drug delivery pump implant.

In patients who do not experience an aura, other sensible symptoms, such as paresthesia of a hand that progresses proximally up the arm signals onset (see, for example, The Merck Manual, 2006, page 1848). In an intellectually impaired patient or a young child, automatic release must be effected by a sensor implant which detects a physiological concomitant and experiential correlate to onset, signals the microcontroller to energize the pump, and provides the quantitative information for controlling the pump. Provided distention or vasoldilation of the extracerebral cranial arteries signals onset, a thin film strain gauge pressure type sensor implant can be used.

If for any reason, the action of the drug produces results outside the target range, further delivery is stopped upon receipt of pertinent sensor feedback. An unanticipated effect can be encountered during preliminary testing or at any time thereafter in which the patient experiences a primary change in metabolism, disease induced or otherwise. Then delivery of the drug is immediately stopped, and if available, a reversal agent (antidote, counteractant) is delivered. Drug delivery cessation and recovery are the reasons for requiring that all pumps be reversible.

The sensors signal out of range values to their morbidity or organ system control node, whereupon a higher-order controller programmed to coordinate the action of the nodes issues the commands to achieve the most efficacious overall response. The application of such a system is generally reserved for chronic conditions where an automatic system not only effects remedial action immediately to interdict progression but serves to dispel a central condition that detracts from the quality of life. Such an automatic ambulatory system, operating barely if at all noticed, has the potential to forestall if not prevent the inducement by a chronic systemic disease of a terminal condition. For example, if left untreated, diabetes, hypertension, atherosclerosis, or the metabolic syndrome will eventually induce chronic, then end-stage kidney disease.

A suitable circumstance where comorbid disease may be best controlled with automatic monitoring by sensor implants and the delivery of insulin and drugs to treat concurrent hypertension with an angiotensin converting enzyme inhibitor and angiotensin receptor blocker, or atherosclerosis with a statin, is diabetic nephropathy. By impeding progression to end-stage renal disease, which necessitates precise diagnosis and correctly measured treatment, survival is extended (see, for example, The Merck Manual 18th edition, 2006, page 2008). The automatic system functions continuously, and can do so in a mentally impaired patient.

Through the use of catheters made of a hydrophilic materials having a slippery internal surface, usually a fluoropolymer such as polytetrafluoroethylene tubing backed up by at least one accessory channel to clean away any buildup of clot, crystal, or biofilm used in accordance with the guidelines set forth in the foregoing and in this application thwarts the use of small gauge synthetic tubing in the body. Additionally, along the vascular tree, an accessory channel (service channel, sideline) attached to the primary or mainline is always provided to allow the targeted and tightly metered addition of an anticoagulant, antiseptic, and/or anti-inflammatory as well as any other fluid medication into the blood or therapeutic fluid passing through the mainline. By substantially avoiding the systemic circulation, the targeted delivery of medication allows use of the drugs at higher concentrations for restricted site specific local application.

An automatic ambulatory prosthetic disorder response system with direct and targetable access to multiple sites of internal disease must coordinate the automatic treatment of these in a synchronized manner while the patient engages in normal activity. Even one, much less a collection of indwelling—meaning temporary, nonimplanted—catheters would disallow this. Imperative for the implementation of a fully implanted therapeutic system, safe, secure, and durable pipeline or electrical conductor to tissue connectors were addressed in the copending applications specified.

Already described in application Ser. No. 14/121,365 are body surface ports and ductus side-entry jackets for connection to tubular anatomical structures, or ductus, to meet the immediate requirement for such connection in an automatic ambulatory prosthetic disorder response control system. However, regardless of application thus, such means overcome the need to detain an otherwise ambulatory patient in the clinic merely because a catheter, infusion line, the tape securing it, or the solution used to promote antisepsis require frequent examination and changing or because more radical surgery necessitates more time to heal.

Described here is a prosthetic disorder response system-compatible fluid and electrical line connector for fastening one or a number of catheters to nontubular internal surfaces and organs, such as the kidneys, the urinary or the gall bladder, the spleen, prostate gland, uterus, and any location along a serous membrane-lined internal surface. Surface ports secure the wound at the body surface, ductus side-entry jackets where connection is made to a tubular anatomical structure, and the internal surface connector described herein is used to attach a catheter to any surface which nontubular, is not articulable by means of encirclement.

### OBJECTS OF THE INVENTION

The central object of the invention is to provide control means over the automatic detection and treatment of disease, the semiautomatic execution of solid organ transplants, and the semiautomatic replacement of congenitally severe malformities of the vasculature so that these procedures will demonstrate much greater than conventional durability.

An object of the invention is to provide a fully implanted automatic diagnostic and therapeutic system to evaluate and treat comorbid disease as well as to detect the emergence of and respond to any of a number of predictable intercurrent diseases immediately upon appearance, before symptoms appear or the patient becomes aware of it, in a patient ambulatory and without a loss in freedom of movement, so that diagnosis and treatment are initiated instantly regardless of the time of day, location, or mental state of the patient.

Another object of the invention is to so devise the system that it can be implanted without the need to interrupt the flow of blood through a vessel treated much less induce circulatory arrest with the complications it risks.

Another object of the invention is to provide such a system to administer the transplantation of a solid organ using the compound bypass method and thereafter, provide automatic and immediate followup treatment thereof, as well as respond to post-transplantation complications and predictable interrurrent disease indefinitely, without detracting from the ambulatory state of the patient.

Another object is to provide the system in the form of a hierarchical control system wherein different disease processes or comorbidities are specifically and simultaneously addressed at the immediate or ground level by sensors that supply output data to a node or controller at the same level in the hierarchy, other nodes dedicated to monitoring different disease processes then passing their data up to a next higher intermediate node for integrating and generating the best response to the combination of disease processes, this pattern of increased comprehension by passage through higher level nodes of integrated data concerning any additional comorbidities finally presented to a master controller programmed to induce and institute the response best calculated to suppress the combination of disease processes and achieve the condition of optimal homeostasis of which the patient is capable.

Yet another object of the invention is to provide a fully implanted system of leak-free, durable, and safe drug and blood catheteric pipelines and electrical devices to provide the implanted microcontroller in monomorbid disease and the microprocessor master controller in comorbid disease immediate access to the diseased nidi or tissues, making it possible to directly pipeline-target therapy to any one organ, gland, or tissue.

Another object of the invention is to make possible the coordination, and usually the collocation, of drug need detection and delivery means so that drugs can be targeted directly to the anatomical point of detection or a point functionally related thereto, thereby enabling the implementation of prosthetic disorder response systems, to include those employing hierarchical control.

Yet another object of the invention is to allow the direct and immediate translation of chemical, electrical, and immunoassay feedback diagnostics into automatic drug delivery around the clock, avoiding any impediment to free movement, whether to the locus of detection, the site of the symptom, and/or the etiological origin, under the control of a hierarchical or complex control system capable of predictive or anticipatory control and further adaptable through ‘learning’ ability, and in so doing, apply such control to the practice of internal medicine.

### DESCRIPTION OF THE PREFERRED EMBODIMENTS OF THE INVENTION

FIG. 1 is a schematic, or nonanatomic, representation of a fully implanted prosthetic disorder response system which includes most of the components found in different applications, not all of which will always be needed in any one system. System components are relegated to an paracorporeal, belt-worn body pack only when these are too large or numerous to be implanted. For simplicity and less expense in competent adult patients, such a system is devised to limit the number and type of functions and components and therefore the complexity of the controller and prescription-program to those best if not necessarily automated.

For pictorial clarity, the mechanism is represented in the much larger form intended for incorporation into a belt-worn body pack; the mechanism substantially the same. When included in a fully implanted system, the pump set and turret mechanism is much smaller in size than if housed in a body pack. It preferred that the system be fully implanted; the representation in FIG. 4 of the system components as having been relegated to a body pack rather than implanted indicates that the sizes and/or number of system components were greater than could be implanted such as may be seen in multiply comorbid disease of congenital pleiotropic genetic origin in younger patients such as trisomy 21 children but otherwise seen in the elderly.

For system simplicity and economy, the release of drugs and/or other therapy such as electrostimulatory for which the need is signaled by the implanted sensors is relegated to the automatic system, whereas scheduled oral medication prescribed for a competent patient is omitted. Automatic response functions include any that demand response to sensor inputs indicating the emergence of an abnormality which had been diagnosed on the basis of a genetic evaluation and the system prepositioned to counteract the condition before the patient became aware of it.

As indicated, for a competent patient, the functions supplied by the system may be supplemented with an oral prescription, for example. FIG. 1 first appeared as FIG. 12A in application Ser. No. 14/998,495, now U.S. Pat. No. 11,013,858, entitled Nonjacketing Side-entry Connectors and pictorial representations and descriptive text explaining the various components used to implement a hierarchical automatic control system and their positioning in the body also appeared earlier in copending application Ser. No. 14/121,365, entitled Ductus Side-entry Jackets and Prosthetic Disorder Response Systems, initially filed on 27 Aug. 2013. Here the control system is singled out, the end connectors and effectors and the fine details of system component materials and mechanical function omitted as duplicative of information already shown.

Accordingly, the focus here is directed to the implementation of these components in the context of an automatic ambulatory disorder response system In FIG. 4, the distinction between system components which are fully, or closed-skin, implanted and others which are relegated to a usually belt-worn body pack is evidence that in this patient, the comorbidities and/or the expressions of each in different tissues were more numerous and complex than could have been diagnosed and responded to by a system comprised of components all of which would have been fully and yet comfortably implanted. For the medical need to preclude the implantation of the entire system should only seldom occur, and when it does, the body pack should be quite small and lightweight.

FIG. 1 provides a schematic, or nonanatomic, overall view of a totally implanted prosthetic disorder response system. In FIG. 1, reproduced from FIG. 12A in U.S. Pat. No. 11,013,858, entitled Nonjacketing Side-entry Connectors and Prosthetic Disorder Response Systems, part number 46 is a subcutaneously positioned surface port with separate openings into each drug storage reservoir or delivery pipline—of which only one representative thereof is shown—through which drugs and miniature cabled devices are introduced and biopsy samples are withdrawn; 47 is a drug storage reservoir. Intended to represent one in a number of comorbidities, the same arrangement would apply were the condition monomorbid with the system managed by a microcontroller rather than a master control microprocessor in comorbid disease.

Part number 48 is a drug delivery pipeline, or drugline; 49 a miniature reversible pump; 50 transdermal charging circuitry; 53 the master controller, or control microprocessor; 54 a rechargeable battery; 58 a body surface port with an outlet to release urine through urine outlet hose 51 into collection bag 59; 61 a nonjacketing side-entry connector that securely connects drugline 48 to the urinary bladder; 62 a nonjacketing side-entry connector that securely connects the bladder to outlet hose 51; and 64 a transdermal, or transcutaneous, battery charging secondary coil.

In FIG. 2, providing drugline and drug reservoir vial switching turrets at the intake and outlet lines of each pump in a pair makes it possible to switch the inlets to either or both jackets to any drug loaded. That is, in FIG. 2, one of the pumps in a pump-pair and jacket set is furnished with turrets at both its intake and outlet to allow any drug delivered through the intake turret to be sent to any jacket in the set. FIG. 2 was first published in nonprovisional application Ser. No. 14/121,365 entitled Ductus Side-entry Jackets and Prosthetic Disorder Response Systems, filed on 25 Aug. 2014 following provisional application Ser. No. 61/959,560 filed on 27 Aug. 2013. application Ser. No. 14/121,365 was then updated in continuation-in-part application Ser. No. 15/998,002, filed on 8 Jun. 2018.

In the arrangement depicted in FIG. 2, one of the pumps in a given pump-pair is used independently. The outlet of the other pump in the pump-pair could be plugged into the intake or outlet turret of the other pump; however, the need for such cross-feeding between pumps in a pair is exceptional. Cross-feeding to pumps belonging to other pump-pair and jacket sets is avoided as needlessly complicated as to invite errors. FIG. 2 shows the right-hand pump in a standardized pump-pair wherein drugline switching using turrets allows any drug or line rotated into alignment with the pump intake by the pump intake line switching means shown as a turret to be delivered through any one line rotated into alignment with the pump outlet line by the pump outlet switching means also shown as a turret but without drug vials for simplicity.

FIG. 2 depicts the side-entry connection jacket at the top left as currently connected to pump 56, pump to turret outlet line 64 indexed, or switched by turret 57 motor 61 to the inline position, with accessory or sideline 11 connected to water-jacket or accessory inlet 10 of that jacket. Water jacket 10 assists in preventing extravasation during plug removal and thereafter serves as the accessory inlet to a jacket accessory channel which allows the directly piped delivery of drugs and maintenance solutions, for example, into the jacket and the ductus it encircles. The lines of the jacket to the top right are not currently indexed to the pump inline positions and are therefore disconnected from pump 56. Pump 56 is continuously adjustable in speed and reversible, allowing outflow to and inflow from either jacket over the range of drug volumetric flow rates without the need to switch to lines of different caliber.

Pump 56 is usually one of a pair, one pump usually connected to the sideline. When more than one pump-pair is present, the connection of these to either jacket is through lines connected to the turret respective of each jacket. Reciprocally, jackets not shown in FIG. 2 may communicate with pump 56. The foregoing degrees of flexibility attest to a potential versatility able to respond to extraordinarily complex medical conditions. This potential capability notwithstanding, pump and jacket relations are ordinarily simple. To prevent air from entering the lines in vascular applications, turrets 57 and 59 omit blank vial positions that would leave a line open-ended; and pumping is stopped once the amount of the infusate has passed so that the free end of the line or hose can be disconnected.

As shown, the left-hand turret lacks a vial and reservoir hose plug in table seen at 58 on the right, indicating that in this application, only the right-hand turret loads drug vials or receives medicated hydrogel or other therapeutic substance reservoir lines or hoses. Were, however, drugs to be supplied from the turret to the left or a tacky medicinal hydrogel, for example, to be recirculated through the closed pump circuit with pump 56 when rotated clockwise, then the turret on the left would be of the same kind as that on the right. If to fill the line then stop or recirculate the gel, a reservoir hose would supply the gel necessary to fill the line. Segments along a line of medicinal or nonmedicinal gel or water can be interposed between segments of the primary medicinal as a way to deliver the primary medicinal in an intermittent manner.

Control of this rotating turret mechanism is one means by which the master controller can position drugs for release to specific targets, alternative embodiments such as miniaturized functionally equivalent. To conserve space, drugs are moved through narrow gauge druglines, often conventional catheters. If the distance to the target makes it necessary, the drug can be diluted or positioned at the head of a column of gel or water. This application concerned with control of a totally implanted disorder response system, the review is necessarily cursory, a more thorough description of drug delivery mechanisms provided in copending application Ser. No. 15/998,002.

FIG. 2 shows one of the two pumps in a pump-pair with switching mechanisms at both the pump intake and outlet to allow the sequential delivery of any drug to the mainline or sideline of any jacket. Accordingly, FIG. 2 shows the right-hand pump in a standardized pump-pair wherein line switching using turrets allows any drug or line rotated into alignment with the pump intake by the pump intake line switching means shown as a turret to be delivered through any one line rotated into alignment with the pump outlet by the pump outlet switching means, also shown as a turret, but again without drug vials for simplicity.

In FIG. 2, crushed tacky hydrogel, drugs, drug hydrogels, and/or wash water for separate consecutive delivery to different jackets are delivered from one of the pumps in a pump-pair through the lines 13 and 11 and side-entry connector 6 of either jacket. Pump outlet flow lines (arms, runs) 11 are connected at intervals about outflow indexing turret plate 57, and pump intake lines 13 are connected at intervals about turret drug vials and/or vials used as drug reservoir hose connectors to pump intake sectional tray consisting of sectional tray 58 and hold-down plate 59. Each turret rotates one inlet vial or line into the in-line position at the same time that it rotates the preceding line out of the in-line position. Lines 13 and 11 are given enough slack that these do not interfere with rotation of the turrets.

Also not shown are accessory channels to deliver an anticoagulant such as a heparin or thrombolytic drip to prevent the accumulation of a residue along the inner wall of the druglines, or of clot when the fluid moved is blood. In FIG. 2, part number 3 is a viscoelastic polyurethane foam jacket lining with surface coated to prevent dissolution essential to prevent compression of the vasa vasora and vasa nervora as would induce atherosclerotic degeneration. Part number 4 a strong jacket outer shell or casing made of polyether ether ketone (PEEK) or another biocompatible nonallergenic material such as gear grade nylon with edges rounded to prevent irriation to surrounding tissue.

Part number 5 is a the outer sealing grommet cap of an eccentric bushing that allows the razor sharp circle cutter, or trepan, at the end of the bushing facing into the jacket, hidden in this view but clearly shown in copending application Ser. No. 15/998,002, entitled Ductus Side-entry Jackets and Prosthetic Disorder Response Systems filed on 25 Aug. 2014, to be rotated and reciprocated to expedite removal from the side of the ductus of a plug of tissue to serve as the ostium of sideline 13 exiting side connector, side-stem, or mainline 6, ordinarily fed by a subsidiary sideline shown here as part number 10.

Jacket side-stem, or side-connector 6 shown here ensheaths drugline 13 leading to the turret aligned drug reservoir vial 58; part number 10 is a side-stem subsidiary takeoff, or sidestem, that ordinarily conveys a drugline or accessory channel; 11 used to empty into accessory channel; 13, the jacket mainline having emerged through side-stem, or side-connector 6, used here as the drugline connecting the jacket to the turret aligned drug reservoir vial 58.

Part number 14 is a spring-hinge which urges the jacket shut but not with a restorative force so great as to prohibit growth in a young child; 15 indicates the position of the joint separating the spring-loaded semicylindrical halves of the jacket opposite to the jacket spring-hinges; 16 schematically represents the subcutaneously implanted body surface port through drugs and therapeutic solutions are replenished regardless of the number or size of the components needed as too numerous or large for the system to be fully implanted so that the internally unaccommodable components had to be relegated to a body pack. Part number 18 schemiatically represents the body surface integument, comprising the skin, subcutaneous fascia, and fat.

Part number 19 points to side-entry jacket and lining through and through slits to allow open exposure of a sufficient area of the vascular adventitia of the encircled artery as essential to preclude the complete enclosure of its nervelets and tiny vessels as would induce atherosclerotic degeneration; 56 is the right hand of two drug delivery pumps, shown here as peristaltic, or of the roller type, for propelling drugs from the drug reservoir 58 and through sideline, or accessory channel 11.

Part number 57 is the drug pump outflow indexing turret plate; 58 is the drug turret drug reservoir or vial storage tray that rotates to index the required drug vial into alignment with line 65 leading to drug pump 56 as the drug pump intake line; 59 is the pump intake drug vial hold-down plate, which along with drug storage vial sectional tray 58, comprises the drug pump intake turret; 60 is the drug inlet turret motor of the right-hand pump shown.

Part number 61 is the drug outlet turret motor for the right-hand pump shown; 62 is the right hand drug turret stile or mounting shaft; 63 is the drug vial hold-down plate retainer cap; 64 is the drug pump outlet line that leads into the sideline or accessory channel 11; 65 is the pump drug intake line from drug storage vial 58; and 69 are fluid line cleanouts. An additional accessory channel feeding into the druglines to drip in an anticoagulant or thrombolytic to prevent the formation of clot is not integral to the mechanism is not shown.

Whereas bedridden patients need drugs to be pumped to the target, in an ambulatory patient able to maintain an upright posture, drug reservoirs can allow the drug to flow down to the target under the force of gravity. Generally, it is simpler and less costly to move expensive drugs through druglines in the form of a diluted continuous column rather than to arrange for a much smaller concentrated amount of the drug to be driven down the drugline ahead of a column of water by the reservoir pump. That is, the control, componentry, expense, and susceptibility to malfunction to provide such drug-water reservoir switching and apportioning are more costly than is the use of narrow gauge druglines and diluted drugs of like dose as were these highly concentrated and positioned ahead of a column of water.

Depending upon the connections made between pumps and jackets, a pump or pump-pair can support one or more side-entry jackets, and more than one pump-pair can support a single jacket. In FIG. 2, the ductus side-entry jackets and lines at the top of the figure are described in detail in copending application Ser. No. 15/998,002 entitled Ductus Side-entry Jackets and Prosthetic Disorder Response Systems where the same drawing figure appears as FIG. 32 and part numbers of the mechanism are identified and explained in greater detail.

When too small to provide the volume of medication required, the standardized drug vial shown in FIGS. 33 thru 36 of copending application Ser. No. 15/998,002 for insertion into a turret drug vial receptacle, represented here as part number 58, serves as the connector attached to the end of a hose from the drug reservoir for engagement in the turret. The vial also provides the initial dose of the drug or another drug preparatory to delivery of the primary drug. A more usual and versatile arrangement is shown here in FIG. 2, wherein one of the pumps in a pump-pair and jacket set is furnished with turrets at both its intake and outlet to allow any drug delivered through the intake turret to be sent to any jacket in the set.

The two jackets represented in FIG. 2 as equal in size and distance from the pump might be placed along the same ductus, or ductus differing not only in size and/or distance from the pumpt but belonging to different bodily systems and therefore be assigned to different arms in the hierarchical control system. This might, for example, consist of a jacket placed along the digestive tract and another placed about the artery that supplies that segment of the tract, or each jacket might treat different diseases whether related or coincidental. Flexibility and speed in reconnection of the lines to and from each pump are often significant when line switching must be reconfigured quickly as might arise in the testing undergone during installation.

Whereas lines supporting side-entry connection jackets placed along the vascular tree or the urogenital tract are small enough in caliber that placement should seldom encroach upon neighboring tissue as to cause pain by compression of a nerve or vessel, larger jackets positioned along the gastrointestinal tract or airway might do so. Where anatomical or operative considerations discourage the placement of multiple lines to access a given jacket, the input line to each jacket is provided with a conventional miniature piggyback port with valve. Encroachment upon neighboring tissue is to be avoided. All jackets have their edges and corners rounded, If necessary, a polymeric gas-permeable cushion not subject to enzymatic or hydrolytic breakdown can be glued to the jacket to serve as a cushion between it and the neighboring anatomy.

FIG. 3 provides a schematic of the control hierarchy for a single pump-pair in support of four jackets in the pump-pair and jacket set, the control program, that is, the prescription-program, of the master node, a microprocessor, determined by the specific or comorbid conditions to be treated. Nodes subordinate to the master node are generally microcontrollers. FIG. 3 provides a schematic of the pump-pack, jacket set, and control system. In FIG. 3, only the control train is represented, the distinction between intra and extracorporeal elements omitted.

A paracorporeal such as a waist worn body pack affords considerably more space and can hold a larger volume of numerous drugs, other therapeutic agents, and equipment maintenance solutions. While depicted with full-sized components as relegated to a body pack, the control hierarchy is always microminiaturized and therefore implantable with the impediment of a pack eliminated. FIG. 3 is a diagrammatic representation of the control train when a single pump-pair and jacket set is implanted or inserted in a pump body pack, shown therefore, in the abstract as to whether the system components are positioned inside or outside the body. As indicated, the control trains in FIGS. 3 and 4 comprise hierarchical control systems.

FIG. 3 provides a schematic of the control hierarchy for a single pump-pair in support of four jackets in the pump-pair and jacket set, the control program, that is, the prescription-program, of the master node, a microprocessor, determined by the specific or comorbid conditions to be treated. Nodes subordinate to the master node are generally microcontrollers. Unlike FIG. 4, in FIG. 3, only the control train is represented, the distinction between intra and paracorporeal elements not indicated. FIG. 4 provides a schematic of the pump-pack, jacket set, and control system.

FIG. 4 is a simplified schematic or circuit diagram of the interconnections among the nodes in a hierarchical control system and the positioning of system components as implanted or outside the body such as when a second pump-pair and jacket set is added to the first in the pump-pack. An extracorporeal pack affords considerably more space and can hold a larger volume of numerous drugs, other therapeutic agents and equipment maintenance solutions, the need therefor mostly applicable to the elderly prescribed polypharma. The control hierarchy itself comprises microcontrollers and a master control microprocessor which tiny, are implanted with the impediment of a pack eliminated. A given hierarchy can be embodied in a single microchip.

When implanted, the contents labeled body pack at the lower left in FIG. 4 are miniaturized; otherwise, FIG. 4 applies no less to a fully implanted as to a body pack carry system. Also when implanted, to preclude complications due to encroachment upon or strangulation of tissue by wires, data intercommunication from the sensors and subordinate nodes and control signals from the master node are preferably by wireless, or Bluetooth transmission. For pictorial clarity, where the electrical and fluid lines between nodes and jackets are actually separate and distinct, those between nodes and jackets are shown as consolidated until finally led to each jacket, and remote sensors and auxiliary drug supply reservoirs have been omitted. Electrical connectors, more remote sensors, drug supply reservoirs and outlet pumps controlled by the master node have been omitted.

FIG. 3 provides a schematic of the control hierarchy for a single pump-pair in support of four jackets consisting of the pump-pair and jacket set, connecting fluid and electrical lines when not wireless, and the subsidiary node microcontrollers, and control microprocessor which as the master node, integrates and coordinates the information received from the subsidiary nodes and administers the prescription-program. The number of channels or arms of nodes is determined by the number of comorbidities to be treated. Nodes subordinate to the master node are generally microcontrollers.

FIG. 4 includes both the component implanted—jackets, sensors, fluid lines, control electronics, and so on—and those relegated to the pump-pack. Depending upon the size and weight the patient is likely to tolerate, a paracorporeal pack affords considerably more space and can hold a larger volume and number of drugs, other therapeutic agents, and equipment maintenance solutions. Not all system components able to be situated outside the body, the impediment of a body pack is to be avoided whenever possible. When implanted, the contents labeled body pack at the lower left in FIG. 4 are miniaturized; otherwise, FIG. 4 applies no less to a fully implanted as to a body pack carry system.

Fluid and electrical connections between the implanted and pack-relegated components are conventional, numerous like situations—ventrical assist devices, artificial hearts—having set the precedent. In FIG. 4, such connections are schematically represented as plugs and sockets that appear much as a square wave. To preclude complications due to encroachment upon or strangulation of tissue by wires, data intercommunication from the sensors and subordinate nodes and control signals (but not power, which is delivered by hard wire or transcutaneous energy transfer to the inmate battery of each end effector) from the master node are preferably wireless, such as by Bluetooth transmission, the respective targets distinguished by carrier frequency.

In FIGS. 3 and 4, single lines are electrical, or if it is found difficult to route the electrical lines without the risk of strangulating intervening structures, then connected by wireless Bluetooth transmission rendered selective by difference in carrier frequency with power transferred to component inmate batteries by transcutaneous energy transfer. If virtually simultaneous operation is essential but cannot be achieved with a single carrier switched among the jackets, then the microprocessor is provided with more than one transmitter.

Further for visual clarity, where the electrical and fluid lines between nodes and jackets are actually separate and distinct, those between nodes and jackets are shown as consolidated until finally led to each jacket, and remote sensors and auxiliary drug supply reservoirs have been omitted. Electrical connectors, more remote sensors, drug supply reservoirs and outlet pumps controlled by the master node have been omitted. If provided with the requisite switching and valving, the fluid and electrical lines shown as shared could support each jacket independently but not simultaneously, the utility thereof contingent upon the condition or conditions to be treated; simultaneous capability is accomplished by furnishing the components necessary.

1. The combination of a closed system of fluid pipelines implanted in the body for directly pipeline-targeting medicinal fluids from implanted drug reservoirs accessed through a subcutaneously implanted body surface port, thence through secure end-connectors into diseased tissue as commanded by a prescription-programmed implanted microelectronic controller taking sensor inputs to detect the need for such therapy at a site of disease, said pipelines also usable to pass miniature diagnostic cabled devices such as scopes, and miniature cabled therapeutic devices such as lasers and thrombectomizers through said subcutaneously implanted body surface port for direct delivery through the lumina of said pipelines to the sites of disease.

2. A system according to claim 1 wherein a control system is organized hierarchically, so that an implanted master control microprocessor programmed with the diagnostic and therapeutic information necessary to treat one in a number of symptoms is able at the lowest level of diagnostic and therapeutic nodes in the hierarchy to evaluate sensory data pertaining to each symptom in comorbidity, this information then passed up to the next level of nodes where the combination of therapeutic measures is optimized to cover each morbidity, this process continued up the levels in the cross-morbidity coordinating hierarchy until the summary data at the penultimate level is passed to the implanted master control microprocessor for execution of its prescription-program to translate the sum of data needed to effectuate the optimal net therapy across the combination of morbidities as will most closely reinstate normal homeostasis.

3. The combination of a system of electrical conductors implanted in the body for energizing electrically powered therapeutic devices such as electrostimulatory and thermal, each device directed toward the same site of disease as a disease analyte sensor respective of each, the output of said sensors passed to a prescription-programmed implanted microelectronic controller to actuate said devices at each site as necessary.

4. The combination of a closed system of fluid pipelines implanted in the body for directly pipeline-targeting medicinal fluids from implanted drug reservoirs accessed through a subcutaneously implanted body surface port, thence through secure end-connectors into diseased tissue, and electrical conductors implanted in the body for energizing electrically powered therapeutic devices such as electrostimulatory and thermal, each device directed toward the same site of disease as the disease analyte sensor respective of each, the data of said sensors passed to a prescription-programmed implanted microelectronic control system controller to pipeline-target medication and actuate said therapeutic devices at each site as commanded by a prescription-programmed implanted microelectronic controller taking sensor inputs to detect the need for medicinal, electrostimulatory, and thermal therapy at each site of disease.

5. A system according to claim 4 wherein said control system with targeting medicinal pipelines and therapeutic devices is organized hierarchically, so that an implanted master control microprocessor programmed with the diagnostic and therapeutic information necessary to treat any symptoms in a number of comorbidities is able at the lowest level of diagnostic and therapeutic nodes in the hierarchy to evaluate sensory data pertaining to each symptom of each morbidity, this information then passed up to the next level of such nodes where the combination of therapeutic measures is optimized to cover both morbidities, this process continued up the levels in the cross-morbidity coordinating hierarchy for as many comorbidities as present until the summary data at the penultimate level is passed to the implanted master control microprocessor to translate the sum of data in accordance with its prescription-program into the net therapy that will most closely approximate normal homeostasis across the combination of morbidities.

6. A system according to claim 4 wherein a plurality of ductus side-entry jackets and nonjacketing side-entry connectors wherewith at least one pump supplying fluid medicinals to these is controlled by a microprocessor according to a prescription program, such that:
A plurality of disease symptom sensors implanted at different locations in the body send outputs as negative feedback that signal out of range conditions to ground level microcontroller nodes in a hierarchical control system;
The microcontroller passes its information up to microcontroller nodes at the next higher level in the hierarchy until at the highest level;
The system master control microprocessor responds according to its prescription-program by returning a response signal down through the reporting chain of successive nodes to cause said pump to index to and release the medication prescribed for the symptom to bring the node output within the normal range,
This process applied to multiple symptoms in comorbid disease to effectuate that action which will achieve the optimal response to each symptom in the sum thereof as best recovers to normal homeostasis.

7. A control system according to claim 4 wherein plural implanted sensors assigned to symptoms attributable to one and the same morbidity transmit their outputs to microcontrollers respective of each for response.

8. A primarily implanted automatic disorder response system that coordinates the data provided by a plurality of implanted sensors directed to a disease process and transmits its data to a microcontroller to determine the drug most efficacious therefor and controls the outlet motor of the drug reservoir storing said drug to release said drug through a catheteric pipeline that isolates as it delivers the drug directly into the blood supply and parenchyma of the affected tissue, thus averting the side effects provoked when said drug is dispersed throughout the circulatory system so that nontargeted tissue is adversely exposed to said drug.

9. A primarily implanted automatic disorder response system that coordinates the data provided by a plurality of implanted sensors, each assigned to an arm directed toward a symptom of one in a plurality of disease processes, these sensors transmitting their data to a microcontroller respective of the disease process aimed at by this set of sensors, other sets of sensors concurrently aimed at other disease processes assigned to other arms of the control system likewise transmitting their data to microcontrollers respective of the disease process to which each set of sensors is aimed, the data then transmitted to the next higher, cross-morbidity level microcontroller nodes to integrate the data sets at the second level in order to determine which drugs in the fewest number and smallest dose and which nondrug effectors such as electrostimulatory and thermal, will optimally affect the combination of symptoms at this second level, these microcontroller nodes then transmitting the results of their integration of the sensory data for the two morbidities to the control nodes at the next higher level, typically, that of a master control microprocessor, which integrates the data from the system arms to actuate the outlet motors of the drug reservoirs to discharge the therapeutic response identified thus through catheteric pipelines and electrical lines to the sites of disease in the proportions that will elicit the optimal effect over the combination of disease processes as will most closely reinstate normal homeostasis.
